(19) United States
(12) Patent Application Publication
(10) Pub. No.: US 2022/0380734 A1
(43) Pub. Date: Dec. 1, 2022

(54) SYSTEMS AND METHODS FOR LUNG CELL EXPANSION AND DIFFERENTIATION

(71) Applicant: DUKE UNIVERSITY, Durham, NC (US)

(72) Inventors: Purushothama Rao Tata, Durham, NC (US); Brigid Hogan, Durham, NC (US); Hiroaki Katsura, Durham, NC (US)

(73) Assignee: DUKE UNIVERSITY, Durham, NC (US)

(21) Appl. No.: 17/764,040

(22) PCT Filed: Sep. 28, 2020
(86) PCT No.: PCT/US2020/053158

§ 371 Date: Mar. 25, 2022

Related U.S. Application Data
(60) Provisional application No. 62/906,241, filed on Sep. 26, 2019.

(51) Int. Cl. C12N 5/071; C12N 5/00

(52) U.S. Cl. CPC C12N 5/0688; C12N 5/0068; C12N 5/0037; C12N 2500/90; C12N 2533/90

(57) ABSTRACT

The present disclosure provides systems for growing and, modeling lung cells in organoid cultures and methods of using same.

### CROSS REFERENCE TO RELATED APPLICATIONS

This application claims priority to U.S. Provisional Patent Application Ser. No. 62/906,241, filed Sep. 26, 2019, the contents of which is hereby incorporated by reference in its entirety.

### FEDERAL FUNDING LEGEND

This invention was made with government support under the National Institutes of Health, National Institute of Allergy and Infectious Diseases Grant Nos. UC6-AI058607, AI132178 and AI149644. The Federal Government has certain rights to this invention.

### STATEMENT REGARDING SEQUENCE LISTING

A computer readable form of the Sequence Listing is filed with this application by electronic submission and is incorporated into this application by reference in its entirety. The Sequence Listing is contained in the file created on Sep. 25, 2020, having the file name “20-1324-WO_Sequence-Listing_SEQ.txt” and is 10 kb in size.

### BACKGROUND

### Field

The present disclosure provides systems and methods for growing lune stem and progenitor cells in organoid cultures and methods of using same.

### Description of the Related Art

Tissue regeneration is orchestrated by the coordinated activities of stem and progenitor cell populations guided by the surrounding milieu. After injury, progenitors' transition from a quiescent to an activated state in which they either rapidly proliferate or differentiate into functional differentiated cells. In some tissues, progenitors generate intermediate transient amplifying cells, which rapidly generate more cells before they undergo differentiation. Multiple factors, within the microenvironment as well as systemic factors are known to dictate the fate of progenitor cells. For example, chronic inflammation, aging, excessive extra cellular matrix (ECM) deposition are frequently associated with defective regeneration, which in some cases leads to tissue degeneration and eventually progress to fibrosis. Therefore, understanding the cell states through which stem and progenitor cells pass in order to repair damaged tissues and the influence of the microenvironment on the trajectories of these cells is of clinical significance.

In the lung, alveolar epithelium maintenance at homeostasis and regeneration after injury is fueled by surfactant-producing cuboidal type-2 alveolar epithelial cell (AEC2), which can self-renew and differentiate into thin, flat, and gas exchanging type-1 alveolar epithelial cells (AEC1). AEC2s also play a key role in providing a first line of defense against viruses, such as the novel coronavirus, SAILS-CoV-2, and pathogens. However, the nature of the pathways that are dysregulated in human AEC2s in response to SARS-CoV-2 infection and how these pathways intersect with other forms of defense mechanisms are not currently known. It is also unclear whether and how AEC2s maintain stem cell characteristics while activating anti-viral defense mechanisms.

Recent studies have identified a subset of AEC2 that are enriched for active wnt signaling and have higher “sternness” compared to neighboring win-inactive AEC2s. Such differences in alveolar progenitor cell subsets, apparently, is due to the differences in microenvironmental signals. In this case, win-active AEC2s are in the vicinity of PDGFRa expressing alveolar fibroblasts, which produces ligands to activate wnt signaling in AEC2s. The conversion of cuboidal AEC2 to thin and extremely flat AEC1 requires dramatic changes to cell shape, structure and mechanical properties. While recent studies have described pathways, including Wnt, BMP, Notch, TGF, YAP, NFkB etc., involved in AEC2 proliferation and differentiation, the transitional cell states through which AEC2 pass during their differentiation into AEC1 has been elusive. In addition, the influence of microenvironmental changes on such transitions is important in the context of defective regeneration. Indeed, recent studies revealed that sustained Notch signaling can block the transition of AEC2s into AEC1.

Elucidating such cell state transitions and the mechanisms that control these processes are largely hindered by the lack of tractable models. While AEC2s can be propagated and differentiated into AEC1 in alveolospheres, the lack of defined conditions either to propagate, maintain or to differentiate AEC2s in organoid or three dimensional cultures or alveolosphere models is limiting these studies.

Organoid cultures derived from adult AEC2s provide the opportunity to address these questions. Current conditions require co-culture of AEC2s with PDGFRa+ fibroblasts isolated from the alveolar stem cell niche or lung endothelial cells isolated from fetal tissues. In addition, current culture media are poorly defined and contains unknown factors derived from fetal bovine or calf serum and bovine pituitary extract. Such complex conditions do not provide a modulate system in which AEC2s can be either selectively expanded or differentiated into AEC's. Therefore, defined culture conditions are needed to study cell type-specific effects and for high throughput pharmaco-genomic studies to discover drugs for treating diseases.

Described herein are chemically defined conditions for lung stem cell expansion, maintenance, and differentiation in ex vivo organoid cultures.

### BRIEF SUMMARY OF THE DISCLOSURE

The present disclosure is based, in part, on the discovery by the inventors of a chemically defined culture system for growth of lung stem cells in 3-dimensional cultures (organoids) that does not require the use of unknown growth components or feeder cells in the culture.

One aspect of the disclosure provide a type 2 alveolar epithelial cell culture medium comprising serum-free medium and an extracellular matrix component, wherein the culture medium is chemically defined and stroma free.

In some embodiments of the disclosure, the scrum-free medium and the extracellular matrix component are mixed at a ratio of about 1:1.

In some embodiments of the disclosure, the extracellular matrix component is matrigel, Collagen Type I, Cultrex reduced growth factor basement membrane, Type R, or human type laminin.

In some embodiments, the serum free medium of the disclosure comprises at least one growth nutrient selected from the group consisting of 0431542, CHIR 99021, BIRB796, Heparin, human EGF, FGF10, Y27632, Insulin-Transferrin-Selenium, Glutamax, B27, N2, HUES, N-acetylcysteine, antibiotic-antimycotic in Advanced DMEM/F12, and combinations thereof.

In some embodiments of the disclosure, the medium is a type 2 alveolar epithelial cell culture expansion medium. In some embodiments of the disclosure, the expansion medium further comprises a cytokine selected from the group consisting of IL-1β, TNFα, and combinations thereof. The IL-1β and TNFα can be from a mouse.

Another aspect of the disclosure provides a type 2 alveolar epithelial cell culture maintenance medium, the maintenance medium comprising the expansion medium of the disclosure, and wherein the maintenance medium further comprises a hone morphogenetic protein (BMP) inhibitor.

In some embodiments of the disclosure, the BMP inhibitor is selected from the group consisting of Noggin, DMH-1, chordin, gremlin, crossveinless, LDN193189, USAG-1 and follistatin, and combinations thereof.

Another aspect of the disclosure provides a type 2 alveolar epithelial cell culture differentiation medium, wherein the differentiation medium comprises at least one of the following growth medium components selected from the group consisting of ITS, Glutamax, Heparin, EFG, FGF10, anti-anti in Advanced DMEM/F12 and/or combinations thereof.

In some embodiment, wherein the differentiation medium comprises serum (e.g., fetal bovine serum or human serum). In other embodiments, the differentiation medium is a serum-free medium.

In some embodiments, the differentiation medium of the disclosure does not contain inhibitors of TGFβ and p38 kinase.

In some embodiments, the differentiation medium of the disclosure comprises IL-6.

Yet another aspect of the disclosure provides a chemically defined and stroma-free organoid culture system for the culturing, expansion, maintenance and/or differentiation of alveolar epithelial cells, the system comprising isolated alveolar epithelial cells cultured in the medium of the disclosure. In some embodiments, the alveolar epithelial cells comprises type 2 alveolar epithelial cells.

Yet another aspect of the disclosure provides a method of expanding, maintaining, and/or differentiating type 2 alveolar epithelial cell in ex vivo organoid cultures, the method comprising obtaining type 2 alveolar epithelial cells and culturing the cells in a medium of any of the disclosure.

in some embodiments of the disclosure, a cytokine is added to the culture medium for about the first four days of culture.

In some embodiments of the disclosure, the type 2 alveolar epithelial cells are expanded in amount sufficient to engraft in a subject. In some embodiments of the disclosure, the type 2 alveolar epithelial cells are harvested and injected into a subject.

In some embodiments of the disclosure, the organoid culture is expanded in an amount sufficient to use for gene editing or lung disease modeling.

Yet another aspect of the disclosure provides a method of culturing lung tumor cells in the absence of fibroblasts, the method comprising isolating tumor cells from a subject, and contacting the tumor cells with the expansion medium of the disclosure.

Yet another aspect of the disclosure provides a method of culturing alveolospheres infected with a pathogen, the method comprising culturing lung cells with the expansion medium of the disclosure and inoculating the lung cells with a pathogen in an amount effective to infect the lung cells.

Yet another aspect of the disclosure a method for identifying an agent capable of treating or preventing pathogen infections in an organoid culture, the method comprising i) culturing the cells in the expansion medium of the disclosure; ii) inoculating the cells with a pathogen in an amount effective to infect the cells; iii) contacting the cells with an agent; and iv) determining whether the agent causes a reduction in the amount of the pathogen in the cells relative to a cell that has not been treated with the agent.

In some embodiments of the above method, step iii is optionally performed before step ii.

In some embodiments of the disclosure, the pathogen is a bacterium (e.g., Bordetella pertussis, Streptococcus pneumonia, Haemophilus influenza, Staphylococcus aureus, Moraxella catarrhalis, Streptococcus pyogens, Neisseria meningitidis, Pseudomonas aeruginosa, or Klebsiella pneumoniae), a virus (e.g., 229E, NL63, OC43, HKU1, HERS-CoV, SARS-CoV, or SARS-CoV-2, an influenza-A virus, an influenza-B virus, or an enterovirus), or fungus (c.a., Aspergillosis).

In some embodiments of the disclosure, the cells are tracheal basal cells, bronchiolar secretory cells, club variant cells, alveolar epithelial progenitor cells, clara variant cells, distal lung progenitors, p63+ Krt5− airway cells, lineage negative epithelial progenitors, bronchioalveolar stem cells, Sox9+p63+ cells, neuroendocrine progenitor cells, distal airway stem cells, submucosal gland duct cell, induced pluripotent stem cell-derived lung stem cells, or alveolar type 2 epithelial.

Yet another aspect of the disclosure provides a method of reducing the viral titers in alveolospheres infected with SARS-CoV-2, the method comprising contacting alveolospheres with an agent before the alveolospheres are exposed to SARS-COV-2, wherein the alveolospheres exhibit reduced viral titers relative to alveolospheres that have not been contacted with the agent.

In some embodiments of disclosure, the agent is an interferon (e.g., IFNα and IFNγ).

Yet another aspect of the present disclosure provides a kit comprising a chemically defined and stroma-free organoid culture system for the culturing, expansion, maintenance and/or differentiation of alveolar epithelial cells, the kit a medium of the disclosure, and instructions for use.

Yet another aspect of the present disclosure provides a kit comprising a chemically defined and stroma-free organoid culture system for determining agents to treat or prevent bacterial, viral and fungal infections in organoid cultures, the kit comprising a medium of the disclosure and instructions for use.

Yet another aspect of the disclosure provides a kit comprising a chemically defined and stroma-free organoid culture system for determining agents to treat or prevent bacterial, viral and fungal infections in organoid cultures or their derivatives ex vivo and in vim, the kit comprising a medium of the disclosure and instructions for use.

### BRIEF DESCRIPTION OF THE DRAWINGS

FIGS. 1A-1C show experiments to test stromal cell dependency in alveolar organoid culture system. FIG. 1A are schematics of organoid cultures to test stromal cell dependency. AEC2s were cultured in Matrigel alone (left) or were cultured in Matrigel alone with stromal cells around the Matrigel with space between them (middle) or were mixed with stromal cells in Matrigel (right). FIG. 1B are representative images of organoid culture in each condition at day 20. FIG. 1C is quantification of colony forming efficiency (CFE) in each condition. Error bas, mean±s.e.m (n=3).

FIGS. 2A-2E show alveolar stem cell niche receptor-ligand interactome guided optimization of medium components for defined conditions for alvcolosphere cultures. FIG. 2A is a schematic of the scRNA-seq experiment. FIG. 2B is a t-distributed stochastic neighbor embedding (t-SNE) visualization of epithelial cells and fibroblasts from mouse alveolosphere culture. Cells are shaded by cluster assignment based on marker genes expression. FIG. 2C shows rSNE plots showing the expression of marker genes in each cluster. Cells are shaded by normalized expression of each gene. FIG. 2D show schematics of the receptor-ligand interactions between AT2s and fibroblasts in alveolosphere culture. FIG. 2E are dot plots showing gene expression of receptors, ligands, and regulators in key signaling pathways in each cluster. Dot size and shading intensity indicate the number of cells expressing the indicated transcript and the expression level, respectively.

FIGS. 3A-3C shows the effect of medium components in organoid growth. FIG. 3A are representative images of alveolospheres in each culture condition. SCE refers to: SB431542, CHIR99021 and EGF without p38 inhibitor (BIRB796). Scale bar, 1 mm. FIG. 3B is a graph showing quantification of CFE in each condition shown in FIG. 2A. Error bars indicates mean±s.e.m. (n=3, at least two wells per condition). FIG. 3C is a graph showing alveolospheres that are greater than 300 μm in perimeter and were quantified in each condition shown in FIG. 3A. SCE vs SCE+p38i, p=1.65×10⁻¹⁰; SCE vs SCE+p38i+FGF7, p=5.47×10⁻¹⁴; SCE vs SCE+p38i+FGF10, p=4.94×10⁻¹⁴; SCE vs SCE+p38i-FGF7_FGF10, p=5.1×10⁻⁶; n.s, not significant; Steel-Dwass test.

FIGS. 4A-4C show establishment of chemically defined stroma-free alveolar organoid culture system. FIG. 4A is a schematic and representative images of organoid culture in MTEC and serum free medium at day 10 and day 15. FIG. 4B is a graph showing quantification of CFE. FIG. 4C is a graph showing organoid size.

FIGS. SA-5C show establishment of chemically defined stroma-free alveolar organoid culture system. FIG. 5A are a schematic and representative images of organoid culture with and without IL-1β/TNFα at day 10 and day 15. FIG. 5B is a graph showing quantification of CFE. FIG. 5C is a graph showing organoid size.

FIGS. 6A-68 show establishment of chemically defined stroma-free alveolar organoid culture system. FIG. 6A is a schematic showing pulse stimulation of IL-1β. FIG. 6B is a graph showing quantification of CFE of the data from FIG. 6A. Error bars, mean±s.e.m (n=3 except for -IL-1β d3 (n=2)).

FIGS. 7A-7D shows characterization of primary human alveolospheres. FIG. 7A is schematic of human alveolosphere culture in SFFF medium. hIL-1β was removed from medium at day 7 and cultured for an additional 7-15 days. FIG. 7B are representative alveolosphere images of three individual donors at day 14. FIG. 7C is a graph showing quantification of colony formation efficiency (CFE). FIG. 7D is a graph showing the size (perimeter) of alveolospheres collected on day 14.

FIGS. 8A-8B show defined conditions for alveolosphere cultures. FIG. 8A are a schematic and representative images of alveolosphere cultures derived from labeled (tdTomato+) in SFFF medium at 10 days and 15 days. FIG. 8B are representative TEM images of alveolospheres cultured in SFFF medium. Scale bar, 2 μm. Higher-magnification image (right) shows lamellar body-like structures. Scale bar, 500 nm.

FIGS. 9A-9B show functional analysis of alveolar organoids in alveo-expansion medium. FIG. 9A is a schematic showing passaging of organoid culture. FIG. 9B is a graph showing a growth curve based on cumulative cell number during passaging in Alveo-Expansion medium.

FIGS. 10A-ION show establishment of a chemically defined human lung alveolosphere culture system. FIG. 10A is a schematic representation of human alveolosphere cultures and passaging in SFFF medium. FIG. 10B are representative images of human alveolospheres from different passages. Scale bar 100 μm. FIG. 10C is a graph showing quantification of the colony formation efficiency of human alveolospheres at different passages. FIG. 10D shows images of immunostaining for SFTPC, SFTPB, and AGER (left panel) or SFTPB, HTII-280 and DC-LAMP (right panel) at P1 and P3 human alveolospheres cultured in SFFF medium for 14 days. FIG. JOE shows images of immunostaining for SFTPC and HTII-280 in cells dissociated from alveolospheres at P2 (top), and P8 (bottom). FIG. 10F is a graph showing quantification of HTII-280⁺SFTPC⁺ cells/total DAPI⁺ cells derived from alveolospheres dissociation from P2 and P8. FIG. 10G are images of bright field (left) and immunostaining for SFTPC, Ki67 and AGER in human alveolospheres at P10. FIG. 10H are graphs showing quantitative RT-PCR for SFTPC and LAMP3 in human alveolospheres at P1 and P6. FIG. 10I are images of immunostaining for SFTPC, and TP63 and SOX2 on alveolosphere sections cultured in SFFF media for 20 days. FIG. 10J are images of immunostaining for NKX2-1, SCGB1A1, and HTII-280 on alveolosphere sections cultured in SFFF media for 20 days. FIG. 10K are immunostaining for AGER and SFTPC in alveolospheres after induction of differentiation by 10% FBS for 10 days. FIG. 10L are images showing immunostaining for AGER and SFTPC on alveolospheres after induction of differentiation by human serum for 10 days. High magnification image (right) shows AGER⁺ cells. Scale bars, 50 μm. Data are presented as mean±s.e.m. FIG. 10M is a schematic representation of human AT2 to AT1 differentiation in alveolospheres. AT2s were cultured in SFFF medium for 10 days followed by culture in ADM for 14 days. FIG. 10N are images of immunostaining for SFTPC and AGER in human alveolospheres cultured in ADM condition for 14 days. Scale bars: B, 100 μm; D, 50 μm; E, 20 μm; H, 20 μm. DAPI shows nuclei in FIG. 3D. FIG. 5E and FIG. 5H. Data are presented as mean±s.e.m.

FIGS. 11A-11I show functional analysis of alveolar organoids in alveo-expansion medium. FIG. 11A is an overview of the gene editing experiment. Overlay of fluorescence and brightfield images of organoids expressing GFP introduced by AAV6-based gene delivery (right). Scale bar, 50 μm. FIG. 11B show schematics of tumor organoid culture. FIG. 11C are representative images of tumor organoids in various media at day 7. FIG. 11D is a graph showing quantification of CFE of tumor organoids at day 5 (right). Error bars, mean±s.e.m (n=3). ***P<0.001. FIG. 11E are images of immunostaining for RAGE. (white), SPC and TOMATO in tumor organoids at day 7. FIG. 11F is a schematic of the grafting experiment. FIG. 11G are representative image of cleared lungs grafted with organoid-derived cells. White dashed line indicates the edge of lung tissue. Scale bar, 1 mm FIG. 11H are representative image of engraftment of organoid-derived cells in the lung. Grafted cells were detected by endogenous TOMATO expression. Scale bar, 100 μm. FIG. 11I are images showing immunostaining for RAGE and SPC of lung section of mice grafted with organoid-derived cells. Grafted cells were detected by endogenous TOMATO expression. Scale bar, 50 μm. Grafting experiment was performed independently three times.

FIGS. 12A-12J shows modulation of cell identities in organoid culture. FIG. 12A is a schematic of the experiment in expansion medium. FIG. 12B are representative whole mount images of organoid in expansion condition at day 10. FIG. 12C are tSNE plots showing the expression of indicated genes, FIG. 12D is a schematic of the experiment in maintenance medium with BMP inhibition. FIG. 11E are representative whole mount images of organoid in maintenance condition at day 10. FIG. 12F are images of immunostaining for SFTPC, Tdt, and AGER (left panel) or SFTPB, Tdt and DC-LAMP (right panel) at P1 and P6 mouse alveolospheres cultured in AMM. FIG. 12G is a schematic representation of mouse alveolosphere passaging. FIG. 12H are representative alveolosphere images at passage 1, 3 and 6. FIG. 12I is a graph showing quantification of CFE at different passages. FIG. 12J are graphs showing quantitative RT-PCR for Sftpc, Abca3 and Lamp3 in mouse alveolospheres at P1 and P6. Asterisks show p<0.05.

FIG. 13 shows representative whole mount images of organoids in Alveo-Expansion (left) and Alveo-Maintenance medium (right) at day 7.

FIGS. 14A-14D shows modulation of cell identities in organoid culture. FIG. 14A is a schematic for organoids in differentiation condition at day 20. FIG. 14B are images showing immunostaining for AGER, SFTPC (left) and HOPX, PDPN (right) in organoids in differentiation condition at day 20. Scale bar, 50 μm. FIG. 14C are images of immunostaining for SFTPC and AGER in mouse alveolospheres cultured in ADM at P1 (left) and P6 (right). Scale bars: D, 1 mm; B and G 50 μm. Data are presented as mean±s.e.m. FIG. 14D show tSNE plots showing the expression of AEC2 markers (Sftpc, Lamp3, Lpcat1) (left) and AEC1 markers (Ager, Hopx, Cav1) (right).

FIGS. 15A-15C shows differentiation of mouse and human AEC2s to AEC1 in cultures with scrum-free differentiation medium. FIG. 15A is a plot showing an enrichment for IL6 transcripts in fibroblasts. FIG. 15B is a schematic showing mouse AEC2s cultured in alveolar expansion medium for 10 days prior to replacing medium with ADM (without serum) supplemented with IL6 (20 ng/mL) and immunofluorescence images (bottom) showing expression of the AEC1 markers AGER. FIG. 15C is a schematic showing human AEC2s cultured in SFFF medium for 14 days prior to replacing medium with ADM (without scrum) supplemented with IL6 (20 ng/mL) and immunofluorescence images (bottom) showing expression of the AEC1 markers AGER.

FIGS. 16A-16E show alveolosphere-derived AT2s express viral receptors and are permissive to SARS-CoV-2 infection. FIG. 16A is a schematic representation for SARS-CoV-2-GFP infection in human alveolospheres. AT2s were cultured on matrigel coated plates in SFFF medium for 10-12 days followed by infection with SARS-CoV-2 virus and RNA isolation or histological analysis after different time points. FIG. 16B are representative wide-field microscopy images from control and SARS-CoV-2-GFP infected human lung alvcolospheres. FIG. 16C is a graph showing viral titers were measured by plaque assays using media collected from lung alveolosphere cultures at 24, 48, and 72 h post infection. FIG. 16D is a graph showing quantitative RT-PCR analysis for SARS-CoV-2 transcripts in control and SARS-CoV-2 infected human AEC alveolospheres. FIG. 16E is a graph showing quantification of SARS-CoV-2 negative strand-specific reverse transcription followed by RT-qPCR targeting two different genomic loci (1202-1363 and 848-981) in Mock and SARS-CoV-2 infected human alveolospheres at 72 h post infection. Asterisks show p<0.05. Scale bars: A, B, and C, 30 μm, D, 20 μm, F, 20 μm. White box in merged image indicates region of single channel images. All quantification data are presented as mean±s.e.m.

FIGS. 17A-17D show transcriptome profiling revealed enrichment of interferon, inflammatory, and cell death pathways in SARS-CoV-2 infected pneumocytes. FIG. 17A is a volcano plot showing upregulated (right) and down-regulated (left) genes in alveolospheres cultured in SFFF infected with SARS-CoV-2. DESeq2 was used to perform statistical analysis. FIG. 17B are graphs showing expression levels of IFN ligands in Mock and SARS-CoV-2 infected human alveolospheres detected by bulk RNA-seq. FIG. 17C are graphs showing expression levels of receptors in Mock and SARS-CoV-2 infected human alveolospheres detected by bulk RNA-seq. FIG. 17D are graphs showing expression levels of downstream targets in Mock and SARS-CoV-2 infected human alveolospheres detected by bulk RNA-seq. Data are presented as FPKM mean±s.e.m.

FIGS. 18A-18E shows that SARS-CoV-2 infection induces loss of surfactants and AT2 cell death. FIG. 18A is a graph showing Quantification of percent of SARS-CoV-2 infected alvcolospheres. FIG. 18B is a graph showing quantification of low infected (1-10 SARS-CoV-2+ cells) and high infected (10 or more SARS-CoV-2+ cells) alveolospheres. FIG. 18C is a graph showing quantification of SFTPC+ cells in uninfected control and SARS− and SARS+ cells in virus infected alveolospheres. FIG. 18D is a graph showing quantification of active-CASP3+ cells in uninfected control (grey), SARS-Cov-2− cells (blue) and SARS-CoV-2+ cells in infected alveolospheres. FIG. 18E is a graph showing quantification of Ki67+ cells in uninfected control (grey), SARS-Cov-2− cells (blue) and SARS-CoV-2+ cells in infected alveolospheres.

FIG. 19 is a dot plot showing cell type specific marker gene expression in epithelial cells obtained from the severe COVID-19 patients.

FIGS. 20A-20B show transcriptome-wide similarities in AT2s from SARS-CoV-2 infected alveolospheres and COVID-19 lungs. FIG. 20A is a volcano plot shows specific genes enriched in AT2 cells in bronchioalveolar lavage fluid from severe COVID-19 patients (right) and AT2s isolated from healthy lungs (control) (left). Wilcoxon rank sum test was used for the statistical analysis. FIG. 20B are violin plots show gene expression of cytokine and chemokine (CXCL10, CXCL14, and IL32), interferon targets (IFIT1, ISG15, and IF6), apoptosis (TNFSF10, ANXA5, and CASP4), surfactant related (SFTPC SFTPD, and NAPSA) and AT2 cell-related (LAMP3, NKX2-1, and ABCA3) in AT2 cells derived from control and severe COVID-19 patient lungs.

FIGS. 21A-21H show IFN treatment recapitulates features of SARS-CoV-2 infection including cell death and loss of surfactants in alveolosphere-derived AT2s. FIG. 21A are representative images of control and IFN-a, IFN-b, IFN-g treated human lung alveolospheres. FIG. 21B is a graph showing quantification of active caspase3+ cells in total DAP1+ (per alveolosphere) cells in control and interferon treated human alveolospheres. FIG. 21C is a graph showing quantification of Ki67+ cells in total DAPI+ cells in control and interferon treated human alveolospheres. *, ** and *** show p<0.05, p<0.01 and p<0.001, respectively. FIG. 21D is a graph showing quantification of RT-PCR analysis for SFTPB in alveolospheres treated with interferons. FIG. 21E is a graph showing quantification of RT-PCR analysis for SFTPC in alveolospheres treated with interferons. FIG. 21F is a graph showing quantification of RT-PCR analysis for ACE2 in alveolospheres treated with interferons. FIG. 21G is a graph showing quantification of RT-PCR analysis for TMPRSS2 in alveolospheres treated with interferons. FIG. 21H are graphs showing quantitative RT-PCR analysis for ACE2 and TMPRSS2 on control and SARS-CoV-2 infected (48 jours pst infection) alveolospheres cultured in SFFF. *, ***, **** show p<0.05, p<0.001 and p<0.0001, respectively.

FIG. 22A is a schematic of IFNs or IFN inhibitor treatment followed by SARS-CoV-2 infection. FIG. 22B are graphs showing viral titers in control, Ruxolitinib-treated, INFa-treated, and IFNg-treated cultures were measured by plaque assay using media collected from alveolosphere cultures at 24 and 48 h post infection.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### Definitions

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-indicated herein, and each separate value is incorporated into the specification as if it were individually recited herein. For example, if a concentration range is stated as 0.1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The term “disease” as used herein includes, but is not limited to, any abnormal condition and/or disorder of a structure or a function that affects a part of an organism. It may be caused by an external factor, such as an infectious disease or chemical toxin, or by internal dysfunctions, such as cancer, cancer metastasis, and the like.

The term “effective amount” or “therapeutically effective amount” refers to an amount sufficient to effect beneficial or desirable biological and/or clinical results.

As used herein, “treatment” or “treating” refers to the clinical intervention made in response to a disease, disorder, or pathogen infection manifested by a patient or to which a patient may be susceptible. The aim of treatment includes the alleviation or prevention of symptoms, slowing or stopping the progression or worsening of a disease, disorder, disease causative agent (e.g., bacteria or viruses), or condition and/or the remission of the disease, disorder or condition.

Unless otherwise defined, all technical terms used herein have the same meaning as commonly understood by one of ordinary skill in the art to which this disclosure belongs.

Chemically Defined, Stroma-Free Organoid Culture Systems

The present disclosure is based, in pail, on the discovery by the inventors of a chemically defined and stroma-free organoid culture system that enables the generation of functional and distinct cell states encompassing alveolar stem cell expansion, maintenance, and differentiation. The chemically defined culture system for growth of lung stem cells in 3-dimensional cultures (organoids) does not require the use of unknown growth components or feeders in the culture.

As used herein, the term “organoid” refers to self-organized three-dimensional (3D) structures or entities that are derived from stem cells grown in culture. Organoids cultures can replicate the complexity of an organ or can express selected aspects of an organ, such as by producing only certain types of cells. Alternatively, at certain stages before differentiation, they can be comprised only of stem cells.

Stem cells are cells that have the ability to both replicate themselves (self-renew) and give rise to other cell types. When a stem cell divides, a daughter cell can remain a stem cell or become a more specialized type of cell, or give rise to other daughters that differentiate into one or more specialized cell types. Two types of mammalian stein cells are: pluripotent embryonic stem cells that are derived from undifferentiated cells present in blastocyst or pre-implantation embryos, and adult stem cells that are found in adult tissues or organs. Adult stem cells can maintain the normal turnover or regeneration of the tissue or organ and can repair and replenish cells in a tissue or organ after damage.

As used herein, the term “stem cell” refers to an undifferentiated cell that is capable of proliferation and self-renewal and of giving rise to progenitor cells with the ability to generate one or more other cell types, or to precursors that can give rise to differentiated cells. In certain cases the daughter cells or progenitor or precursor cells that can give rise to differentiated cells. In certain cases the daughter cells or progenitor or precursors cells can themselves proliferate and self-renew as well as produce progeny that subsequently differentiate into one or more mature cell types.

A progenitor cell refers to a cell that is similar to a stem cell in that it can either self-renew or differentiate into a differentiated cell type, but a progenitor cell is already more specialized or defined than a stem cell.

Stems cells of the present disclosure can be derived from any animal, including but not limited to, human, mouse, rat, rabbit, dog, pig, sheep, goat, and non-human primates.

The stem cells that can be cultivated by the organoid culture system of the present disclosure can be normal (e.g., cells from healthy tissue of a subject) or abnormal cells (e.g., transformed cells, established cells, or cells derived from diseased tissue samples).

In some embodiments, an organoid culture of the present disclosure can be derived from lung stem cells. Division of lung stem cells can promote renewal of the lung's structure. Examples of lung stem cells include, but are not limited to tracheal basal cells, bronchiolar secretory cells (also known as club cells or Clara cells), club variant cells, alveolar epithelial progenitor (AEP) cells, clara variant cells, distal lung progenitors, p63+ Krt5− airway cells, lineage negative epithelial progenitors, bronchioalveolar stem cells (BASCs), Sox9-4 p63+ cells, neuroendocrine progenitor cells, distal airway stem cells, submucosal gland duct cell, induced pluripotent stem cell-derived lung stem cells and alveolar type 2 epithelial (referred to herein as AEC2 or AT2) cells.

In some embodiments, the organoid culture contains alveolar type 2 cells. AEC2 cells can both self-renew and act as progenitors of alveolar type 1 epithelial cells (AEC1). AEC2 cells can replenish the AEC1 cell population under both steady-state and injury conditions. In three-dimensional (3D) (organoid) culture, AEC2 cells can form alveolospheres containing cells that express AEC2 cell markers (e.g., Sftpc, Sfrpb, Lamp3, Lpcat7, HTII-280) and cells that express AEC1 cell markers (e.g., Ager (RAGE), Hopx, and Cav1) and/or cells that express transitional state markers.

In some embodiments, an organoid culture of the present disclosure can be derived from basal stem cells from organs including, skin, mammary gland, esophagus, bladder, prostate, ovary, and salivary glands.

Accordingly, one aspect of the present disclosure provides a cell culture medium comprising, consisting of, or consisting essentially of scrum-free medium and an extracellular matrix component, wherein the cell culture medium is chemically defined and stroma free.

The cell culture media of the present disclosure can be used to culture a number of different cells. In some embodiments, the cell culture medium is a stem cell culture medium. In some embodiments, the cell culture medium is a lung stem cell culture medium. In some embodiments, the cell culture medium is an alveolar type 2 cell culture medium. In some embodiments, the cell culture medium is a tumor cell culture medium (e.g., lung tumor cell). In some embodiments, the cell culture medium is an cell culture medium for a cell that is infected with a pathogen.

The term “cell culture medium” as used herein refers to a liquid, semi-liquid, or gelatinous substance containing nutrients in which cells or tissues can be cultivated (e.g., expanded, maintained, or differentiated).

The term “chemically defined medium” as used herein refers to a medium in which all of the chemicals used in the medium are known and no yeast, animal, or plant tissue are present in the medium. A chemically defined medium can have known quantities of all ingredients.

A “stroma free” cell culture medium as used herein refers to a cell culture medium that does not contain stromal cells or stromal connective tissue. Examples of stroma cells (which may be living or fixed) include, but are not limited to, immune cells, bone marrow derived cells, endothelial cells, pericytes, smooth muscle cells and fibroblasts.

The term “extracellular matrix component” or “ECM” refers to a cell culture medium ingredient that provides structure and biochemical support to surrounding cells. An extracellular matrix component can contain an interlocking mesh of fibrous proteins and glycosaminoglycans. An extracellular matrix component of the present disclosure can comprise proteoglycans (e.g., heparan sulfate, chondroitin sulfate, keratin sulfate), hyaluronic acid, proteins, collagen (e.g., fibrillar (Type I, II, III, V. XI), FACIT collagen (Fibril Associated Collagens with Interrupted Triple helices) (Type IX, XII, XIV, XIX, XXI collagen and collagen type XXII alpha 1), short chain (collagen Type VIII and X), basement membrane (collagen Type IV), and Type VI, VII, XII collagen), elastin, fibronectin, entactin, or laminin. The extracellular matrix component used in the culture medium described here can be a gelatinous protein mixture that is secreted by Engelbreth-Holm-Swarm (EHS) mouse sarcoma cells. Examples of an extracellular matrix component include, but are not limited to, Matrigel™, Collagen Type I, Cultrex reduced growth factor basement membrane, Type R, or human type laminin. In some embodiments, the extracellular matrix component is Matrigel. In other embodiments, the extracellular matrix component is Matrigel from BD Biosciences (San Jose, Calif.) #354230.

The term “scrum-free medium” or SFM refers to medium containing one or more growth nutrients that are capable of supporting the growth of a specific cell type in the absence of serum (e.g., the protein-rich fluid that is separated from coagulated blood). The advantages of using a scrum-free medium include improved consistency between cell culture batches, each batch of cell culture medium does not need to be tested for quality assurance before use, decreased risk of pathogen contamination, improved reproducibility of cell culture studies, and improved isolation and purification of cell culture products.

The term “growth nutrients” of the serum-free medium can comprise a variety of ingredients, such as small molecule compounds (e.g., SB431542, CHIR99021, BIRB796, DMH-1, or Y-27632), recombinant proteins (e.g., Human EGF, Mouse FGF10, Mouse IL-1β, or Mouse Noggin), supplements (e.g., Heparin, N-2, B-27 supplement, Antibiotic-Antimycotic, HEPES, GlutaMAX, or N-Acetyl-L-Cysteine, growth factors, enzyme inhibitor (e.g., trypsin inhibitors), essential vitamins, neuropeptides, neurotransmitters and trace elements (e.g., copper, manganese, zinc, and selenium).

In some embodiments, the serum-free medium can comprise a TGF-β inhibitor. Examples of TGF-β inhibitors include, but are not limited to, LTBPs (latent TGF-β binding proteins), A 77-01, A 83-01, AZ 12799734, D 4476, Galunisertib, GW 788388, IN 1130, LY 364947, R 268712, SB 505124, SB 525334, SD 208, SM 16, ITD 1, SIS3, N-Acetylpuromycin, SB431542, RepSox, and LY2109761.

In some embodiments, the serum-free medium can comprise a GSK3 inhibitor. Examples of GSK-3 inhibitor include, but are not limited to, CHIR 99021, LiCl2, AT7519, CHIR-98014, TWS119, Tideglusib, SB415286, BIO, SB216763, AZD2858, AZD1080, AR-A014418, TDZD-8, LY2090314, 2-D08, BIO-acetoxime, IM-12, 1-Azakenpaullone, or 6-bromoindirubin-3′-oxime.

In some embodiments, the serum-free medium can comprise a p38 MAP kinase inhibitor. Examples of p38 MAP kinase inhibitors include, but are not limited to, S13202190, BIRB796, PD 169316, and SB203580.

In some embodiments, the serum-free medium can comprise an anticoagulant (blood thinner). Examples of anticoagulant include, but are not limited to, heparin or warfarin.

In some embodiments, the scrum-free medium can comprise one or more growth factors. Examples of growth factors include, but are not limited to, epidermal growth factor (EGF), basic fibroblast growth factor (bFGF), fibroblast growth factors (FGF) (e.g., FGF1, FGF2, FGF3, FGF4, FGF5, FGF6, FGF7, FGF8, FGP9, FGF10, FGF11, FGF12, FGF13, FGF14, FGF15, FGF16, FGF17, FGF18, FGF19, FGF20, FGF21, FGF22, FGF23), insulin-like growth factor (IGF) (e.g., IGF-1, IGF-2), platelet derived growth factor (PDGF), nerve growth factor (NGF), granulocyte-macrophage colony stimulating factor, transferrin, stem cell factor (SCF), vascular endothelial growth factor (VEGF), transforming growth factor-alpha (TGF-alpha), brain-derived neurotrophic factor (BDNF), and transforming growth factor-beta (TGF-beta). Growth factors or hormones for use in scrum-free medium can be purified from plants or animals or produced in bacteria or yeast using recombinant DNA technology.

In some embodiments, the scrum-free medium can comprise a ROCK. (Rho kinase) inhibitor. Examples of ROCK inhibitors include, but are not limited to, Y27632, Ripasudil (K-115), Netarsudil (AR-13503), RKI-18, and RKI-11.

In some embodiments, the scrum-free medium can comprise a basal medium supplement or base medium. Examples of basal medium supplements include, but are not limited to, Insulin-Transferrin-Selenium and Advanced DMEM/F12 (Dulbecco's Modified Eagle Medium/Ham's F-12). It will be understood that the culture media of the present disclosure are scalable and the volume of the media can be adjusted according to the culture size.

In some embodiments, the serum-free medium can comprise a substitute for L-glutamine. Examples of a substitute for L-glutamine include, but are not limited to, Glutamax, L-alanyl-L-glutamine (AlaGln), and GlutaminePlus.

In some embodiments, the serum-free medium can comprise a neuronal cell culture component. Examples of a neuronal cell culture component include, but are not limited to, B-27.

In some embodiments, the serum-free medium can comprise a buffer. A buffer is a component of the cell culture medium that can maintain a physiological pH4 (e.g., about 7.2 to about 7.6) Examples of buffers suitable for use in a cell culture medium of the present disclosure include, but are not limited to, HEPES, sodium bicarbonate, and phenol red.

In some embodiments, the serum-free medium can comprise an antioxidant. Examples of antioxidants suitable for use in a cell culture medium of the present disclosure include, but are not limited to, N-acety-L-cysteine, ascorbic acid, and vitamin C.

In some embodiments, the serum-free medium can comprise an antibiotic. Examples of antibiotics suitable for use in a cell culture medium of the present disclosure include, but are not limited to antibiotic-antimycotic, pen/strep, and gentamicin.

In some embodiments, the scrum-free medium can comprise at least one growth nutrient selected from the group consisting of SB431542, CHIR 99021, BIRB796, Heparin, EGF (e.g., human EGF, mouse EGF), FGF10, Y27632, Insulin-Transferrin-Selenium, Glutamax, B27, N2, HEPES, N-acetylcysteine, antibiotic-antimycotic in Advanced DMEM/12 (Dulbecco's Modified Eagle Medium/Ham's F-12), and combinations thereof.

In some embodiments, the serum-free medium and the extracellular matrix component of the cell culture medium are mixed at a ratio of about 1:1.

In some embodiments, the lung stem cell (e.g. type 2 alveolar epithelial cell) culture medium comprises, consists of, or consists essentially of a 1:1 mixture of a serum-free media and a Matrigel, the serum-free media comprising concentrations of 5 μM to 20 μM of SB431542, 1 μM to 10 μM of CHIR 9902, 0.5 μM to 5 μM of BIRB796, 2.5 μg/ml to 20 μg/ml of Heparin, 5 ng/ml to 50 ng/ml of EGF, 5 ng/ml to 10 ng/ml of FGF10, 5 nM to 20 nM of Y27632, insulin-Transferrin-Selenium (1.7 μM of Insulin, 0.068 μM of Transferrin, and 0.038 μM of Selenium), 0.5% to 2% of Glutamax, 1% to 3% of B27, 0.5% to 2% of N-2, 10 mM to 20 mM of HEPES, 0.75 mM to 2 mM of N-acetylcysteine, and 0.5% to 2% of anti-anti, wherein all of these components are contained in Advanced DMEM/F12 base medium, and wherein the medium is stroma free.

In some embodiments, the lung stem cell (e.g. type 2 alveolar epithelial cell) culture medium comprises, consists of, or consists essentially of a 1:1 mixture of a scrum-free medium and a Matrigel, the serum-free medium comprising concentrations of about 10 μM of SB431542, 3 μM of CHIR 9902, 1 μM of BIRB796, 5 μg/ml of Heparin, 50 ng/ml of EGF, 10 ng/ml of FGF10, 10 nM of Y27632, Insulin-Transferrin-Selenium (1.7 μM of Insulin, 0.068 μM of Transferrin, and 0.038 μM of Selenium), 1% of Glutamax, 2% of 1327, 1% of N-2, 15 mM of HEPES, 1.25 mM of N-acetylcysteine, and 1% of anti-anti in Advanced DMEM/F12, and wherein the medium is stroma free.

Another aspect of the present disclosure provides a lung stem cell (e.g. a type 2 alveolar epithelial cell) culture expansion medium. The term “expansion medium” or “serum-free, feeder-free” or “SFFF” as used herein interchangeably and refer to a cell culture medium that can support the proliferation and expansion of stem cells cx vivo.

An expansion medium of the present disclosure can comprise a serum-free medium and an extracellular matrix component, wherein the culture medium is chemically defined and stroma free, and wherein the expansion medium further comprises one or more cytokines.

Cytokines are small proteins (e.g., about 5-20 kDa) that can play a role in cell signaling. Examples of cytokines include, but are not limited to interleukin-1α (IL-1α), interleukin-1β (IL-1β), interleukin-2 (IL-2), interleukin-3 (IL-3), interleukin-4 (IL-4), interleukin-5 (IL-5), interleukin-6 (IL-6), interleukin-7 (IL-7), interleukin-8 (IL-8), interleukin-9 (IL-9), interleukin-10 (IL-10), interleukin-11 (IL-11), interleukin-12 (IL-12), interleukin-13 (IL-13), interleukin-14 (IL-14), interleukin-15 (IL-15), interleukin-16 (IL-16), interleukin-17 (IL-17), interleukin-17 (IL-18), INF-α, INF-β, INF-γ, and tumor necrosis factor-α (TNF-α).

In some embodiments, the expansion medium comprises a cytokine that is selected from the group consisting of IL-1β, TNFα, and/or combinations thereof. In some embodiments, the expansion medium comprises a mouse IL-1β. In other embodiments, the expansion medium comprises a mouse TNFα.

In some embodiments, the expansion medium comprises IL-1β at a concentration of about 0.1 ng/mL to about 10 ng/mL. In some embodiments, the expansion medium comprises IL-1β at a concentration of about 10 ng/ml.

In some embodiments, the expansion medium comprises TNFα at a concentration of about 0.1 ng/mL, to about 10 ng/mL. In some embodiments, the expansion medium comprises TNFα at a concentration of about 10 ng/ml.

In some embodiments, the SFFF medium comprises, consists of, or consists essentially of SB431542, CHIR99021, BIRB796, Y-27632, Human EGF, Mouse FGF10, Mouse IL-1β, Heparin, B-27 supplement, Antibiotic-Antimycotic, HEPES, GlutaMAX, N-Acetyl-L-Cysteine, and a base medium of Advanced DMEM/F12.

In some embodiments, the SFFF medium comprises, consists of, or consists essentially of about 10 μM of S13431542, about 3 μM of CHIR99021, about 1 μM of BIRB796, about 10 μM of Y-27632, about 50 ng/ml of Human EGF, about 10 ng/ml of Mouse FGF10, about 10 ng/ml of Mouse IL-1B, about 5 μg/ml of Heparin, about 1× of B-27 supplement, about 1× of Antibiotic-Antimycotic, about 15 mM of HEPES, about 1× of GlutaMAX, and about 1.25 mM of N-Acetyl-L-Cysteine in a base medium of Advanced DMEM/F12.

In other embodiments, the SFFF medium comprises, consists of, or consists essentially of SB431542, CHIR99021, BIRB796, Y-27632, Human EGF, Human FGF10, Heparin, B-27 supplement, Antibiotic-Antimycotic, HEPES, GlutaMAX, and N-Acetyl-L-Cysteine in a base medium of Advanced DMEM/F12.

In other embodiments, the SFFF medium comprises, consists of, or consists essentially of about 10 μM of SB431542, about 3 μM of CHIR99021, about 1 μM of BIRB796, about 10 μM of Y-27632, about 50 ng/ml of Human EGF, about 10 ng/ml of Human FGF10, about 5 μg/ml of Heparin, about 1× of B-27 supplement, about 1× of Antibiotic-Antimycotic, about 15 mM of HEPES, about 1× of GlutaMAX, and about 1.25 mM of N-Acetyl-L-Cysteine in a base medium of Advanced DMEM/F12.

In some embodiments, the expansion medium is formulated for human lung stem cell (e.g., human AEC2 cells) self-renewal.

It will be understood that some growth nutrients can be added to a culture medium of the present disclosure at different times and for different durations during the treatment period. The treatment period refers to the period of time during which the stem cells are in contact with the culture medium.

In some embodiments, one or more growth nutrients are present in the expansion medium at all times for the duration of the treatment period. Examples of growth nutrients that can be present at all times in the expansion medium include SB431542, CHIR99021, BIRB796, EGF, FGF10, Heparin, B-27 supplement, Antibiotic-Antimycotic, HEPES, GlutaMAX, and/or N-Acetyl-L-Cystein.

In some embodiments, one or more growth nutrients are present in the expansion medium for a limited duration of the treatment period (e.g., from 0 days to 4 days or for just the first 4 days of culture). In some embodiments, a ROCK inhibitor (e.g., Y-27632) is present in the expansion medium from 0 days to 4 days of the treatment period. In some embodiments, a cytokine (e.g., IL-1β) is present only during the first 4 days of the treatment period.

The terms “expansion.” “expand.” or “increase” when used in the context of lung stem cell expansion, means an increase in the number of lung stem cells (e.g., AEC2 cells) by a statistically significant amount. The terms “expansion,” “expand,” or “increase” means an increase, as compared to a control or reference level, of at least about 10%, of at least about 15%, of at least about 20%, of at least about 25%, of at least about 30%, of at least about 35%, of at least about 40%, of at least about 45%, of at least about 50%, of at least about 55%, of at least about 60%, of at least about 65%, of at least about 70%, of at least about 75%, of at least about 80%, of at least about 85%, of at least about 90%, of at least about 15%, or up to and including a 100%, or at least about a 2-fold, or at least about a 3-fold, or at least about a 4-fold, or at least about a 5-fold, at least about a 6-fold, or at least about a 7-fold, or at least about a 8-fold, at least about a 9-fold, or at least about a 10-fold increase, or any increase of 10-fold or greater, as compared to a control or reference level. A control/reference sample refers to a population of cells obtained from the same biological source that has, for example, not been expanded using the expansion medium or methods described herein, e.g., at the start of the expansion medium culture or the initial number of cells added to the expansion medium culture.

Another aspect of the present disclosure provides a lung stem cell (e.g., a type 2 alveolar epithelial cell) culture maintenance medium. The term “maintenance medium” or “AMM” are used herein interchangeably and refer to a cell culture medium that can maintain a particular cell state of a cell in the cell culture. For example, a maintenance medium of the present disclosure can be used to maintain AEC2 cell identity while repressing the induction of AEC1 cells in these organoids.

In some embodiments, a maintenance medium of the present disclosure comprises, consists of, or consists essentially of an expansion medium of the present disclosure and a bone morphogenetic protein (BMP) inhibitor.

Examples of BMP inhibitors include, but are not limited to, Noggin, DMH-1, chordin, gremlin, crossveinless, USAG-1, LDN193189, follistatin, Follistatin-like, DMH-2, LDN 212854, LDN 214117, Dorsomorphin dihydrochloride, and combinations thereof. In some embodiments, the maintenance medium comprises a BMP inhibitor, wherein the BMP inhibitor is noggin or DMH-1. In some embodiments, the Noggin is a mouse Noggin.

In some embodiments, the maintenance medium of the present disclosure comprises Noggin at a concentration of about 1 ng/ml to about 10 ng/ml. In some embodiments, the maintenance medium of the present disclosure comprises Noggin at a concentration of about 10 ng/ml.

In some embodiments, the maintenance medium of the present disclosure comprises DMH-1 at a concentration of about 0.1 μM to about 5 μM. In some embodiments, the maintenance medium comprises DMH-1 at a concentration of about 1 μM.

In some embodiments, the BMP inhibitor is present in the maintenance medium for the entire duration of the treatment period.

In some embodiments, the AMM medium comprises SB431542, CHIR99021, BIRB796, DMH-1, Y-27632, Human EGF, Mouse FGF10, Mouse IL-1β, Mouse Noggin, Heparin, B-27 supplement, Antibiotic-Antimycotic, HEPES, GlutaMAX, and N-Acetyl-L-Cysteine in a base medium of Advanced DMEM/F12.

In some embodiments, the AMM medium comprises, consists of, or consists essentially of about 10 μM of SB431542, about 3 μM of CHIR99021, about 1 μM of BIRB796, about 1 μM of DMH-1, about 10 μM of Y-27632, about 50 ng/ml of Human EGF, about 10 ng/ml of Mouse FGF10, about 10 ng/ml of Mouse IL-1β, about 10 ng/ml of Mouse Noggin, about 5 μg/ml of Heparin, about 1× of B-27 supplement, about 1× of Antibiotic-Antimycotic, about 15 mM of HEPES, about 1× of GlutaMAX, and about 1.25 mM of N-Acetyl-L-Cysteine in a base medium of Advanced DMEM/F12.

In some embodiments, the maintenance medium is formulated for human lung stem cell (e.g., human AEC2 cells) maintenance.

Another aspect of the present disclosure provides a lung stem cell (e.g. a type 2 alveolar epithelial cell) culture differentiation medium. The term “differentiation medium” or “ADM” as used herein interchangeably and refer to a cell culture medium that can promote a particular cell state of a cell to differentiate into a different cell state of a cell in the cell culture. For example, a differentiation medium of the present disclosure can be used to convert AEC2 cells to AEC1 cells.

A differentiation medium of the present disclosure can comprise one or more growth factors and supplements. Furthermore, a differentiation medium of the present disclosure can contain scrum (e.g., fetal bovine serum, human scrum).

A differentiation medium of the present disclosure can comprise a 1:1 mixture of the differentiation medium and an extracellular component (e.g., Matrigel).

In some embodiments, the differentiation medium comprises, consists of, or consist essentially of at least one of ITS, Glutamax, Heparin, EFG, FGF10, Serum (e.g., fetal bovine serum or human serum), and anti-anti in a base medium of Advanced DMEM/F12 and/or combinations thereof.

In some embodiments, the differentiation medium comprises concentrations of ITS of about insulin 1.7 μM, Transferrin 0.068 μM, and Selenite: 0.0381M, about 1% of Glutamax, about 5 μg/ml Heparin, about 5 ng/ml human EFG, about 1 ng/ml mouse FGF10, about 10% Fetal Bovine Serum, and about 1% anti-anti (anti-bacterial and anti-fungal) in a base medium of Advanced DMEM/F12.

In some embodiments, the differentiation medium comprises Human EGF, Mouse FGF10, Heparin, B-27 supplement, Antibiotic-Antimycotic, GlutaMAX, N-Acetyl-L-Cysteine, and Fetal Bovine Serum in a base medium of Advanced DMEM/F12.

In some embodiments, the differentiation medium comprises about 5 ng/ml of Human EGF, about 1 ng/ml of Mouse FGF10, about 5 μg/ml of heparin, about 1× of B-27 supplement, about 1× of Antibiotic-Antimycotic, about 1× of GlutaMAX, about 1.25 mM of N-Acetyl-L-Cysteine, about 10% of FBS in a base medium of Advanced DMEM/F12.

In some embodiments, the differentiation medium comprises Human EGF, Human FGF10, Heparin, B-27 supplement, Antibiotic-Antimycotic, GlutaMAX, N-Acetyl-L-Cysteine, N-Acetyl-L-Cysteine, and Human serum in a base medium of Advanced DMEM/F12.

In some embodiments, the differentiation medium comprises about 5 ng/ml of Human EGF, about 1 ng/ml of Human FGF10, about 5 μg/ml of Heparin, about 1× of B-27 supplement, about 1× of Antibiotic-Antimycotic, about 1× of GlutaMAX, about 1.25 mM, and about 10% of human serum in a base medium of Advanced DMEM/F12.

In some embodiments, the growth nutrients of the differentiation medium are present in the differentiation medium for the entire duration of the treatment period.

In some embodiments, the differentiation medium does not contain inhibitors of TGFβ and p38 kinase.

In some embodiments, the differentiation medium is formulated for human lung stem cell (e.g., human AEC2 cells) differentiation.

In some embodiments, a differentiation medium of the present disclosure does not contain serum (fetal bovine scrum or human serum) and is thus considered a serum-free medium.

A serum-free differentiation medium of the present disclosure can comprise a cytokine instead of serum. In some embodiments, a serum-free differentiation medium of the present disclosure can comprise IL-6 at a concentration of about 10 ng/ml to about 50 ng/ml. In some embodiments, a serum-free differentiation medium of the present disclosure comprises IL-6 at a concentration of about 20 ng/ml.

In some embodiments, a serum-free differentiation medium of the present disclosure can be used to culture lung stem cells (e.g., AEC2 cells) after the lung stem cells have been cultured in a maintenance medium or after the lung stem cells have been cultured in SFFF medium of the present disclosure.

Another aspect of the present disclosure provides a chemically defined and stroma-free organoid culture system for the culturing, expansion, maintenance and/or differentiation of alveolar epithelial cells, the system comprising isolated alveolar epithelial cells cultured in any of the media of the present disclosure.

In some embodiments of the system, the alveolar epithelial cells comprise type 2 alveolar epithelial cells. In other embodiments of the system, the alveolar epithelial cells comprise a mixture of AEC2 and AEC1 cells. In other embodiments of the system, the alveolar epithelial cells comprise predominately (e.g., greater than 50%, 60%, 70%, 80%, 90%, or 99%) AEC2 cells in the culture medium at any given time. In other embodiments of the system, the alveolar epithelial cells comprise predominately (e.g., greater than 50%, 60%, 70%, 80%, 90%, or 99%) AEC1 cells following treatment of AEC2 cells with a differentiation medium.

Methods

Yet another aspect of the present invention provides a method of expanding, maintaining, and/or differentiating lung stem cells in ex vivo organoid cultures, the method comprising, consisting of, or consisting essentially of obtaining lung stem cells and contacting the cells with a culture medium of the present disclosure.

The term “obtaining lung stem cells” refers to the process of removing a cell or population of cells from a subject or lung sample in which it is originally present. Lung stem cells can be obtained from healthy or diseased lung tissue in a living or deceased subject. Lung stem cells can be obtained from subjects that have a disease (lung disease or otherwise) or from subjects who are at risk of developing a lung disease. The cell or population of cells can be separated and purified from other types of cells or tissue from the sample before the lung stem cells are placed in contact with a culture medium of the present disclosure.

In some embodiments of the above method, the lung stem cells comprise tracheal basal cells, bronchiolar secretory cells (also known as club cells or Clara cells), club variant cells, alveolar epithelial progenitor (AEP) cells, clara cells, clara variant cells, distal lung progenitors. p63+ Krt5− airway cells, lineage negative epithelial progenitors, bronchioalveolar stem cells (BASCs), Sox9+p63+ cells, neuroendocrine progenitor cells, distal airway stem cells, submucosal gland duct cell, induced pluripotent stem cell-derived lung stem cells and alveolar type 2 epithelial (AEC2) cells. In some embodiments, the lung stem cells comprise alveolar type 2 epithelial (AEC2) cells.

In some embodiments of the above method, the culture medium is an expansion medium, a maintenance medium, or a differentiation medium of the present disclosure.

In some embodiments of the above method, a cytokine is added to the culture medium for about the first four days of culture.

In some embodiments, the expansion medium, the maintenance medium, or the differentiation medium is formulated for use with human stem cells.

In some embodiments of the above method, the lung stem cells are administered to a subject. In some embodiments of the above method, the lung stem cells are administered to a subject in a therapeutically effective amount.

The term “administration” or “administering” as it applies to a human, primate, mammal, mammalian subject, animal, veterinary subject, placebo subject, research subject, experimental subject, cell, tissue, organ, or biological fluid, refers without limitation to contact of an exogenous ligand, reagent, placebo, small molecule, pharmaceutical agent, therapeutic agent, diagnostic agent, or composition to the subject, cell, tissue, organ, or biological fluid, and the like. Administration can refer, e.g., to therapeutic, pharmacokinetic, diagnostic, research, placebo, and experimental methods. “Administration” also encompasses in vitro and ex vivo treatments, e.g., of a cell, by a reagent, diagnostic, binding composition, or by another cell.

Lung stem cells (e.g., AEC2 cells) cultured by the systems and methods of the present disclosure can be administered to a subject (e.g., a human, mouse, monkey, or any mammal that has lungs) by any route known in the art, including but not limited to, intracerebroventricular, intracranial, intra-ocular, intracerebral, intraventricular, intratracheally, and intravenous.

In some embodiments of the above method, the desired lung stem cells can be expanded in vitro using the expansion medium of the present disclosure to obtain a sufficient number of cells required for therapy, research, or storage (e.g., via cryopreservation). In some embodiments, the desired lung stem cells can be expanded in amount sufficient to harvest, inject, and/or engraft in a subject (e.g. a human, mouse, or any mammal that has lungs).

In some embodiments of the above method, the organoid culture can be expanded in amount sufficient to use for gene editing or lung disease modeling.

Another aspect of the present disclosure provides a method of culturing lung tumor cells in the absence of fibroblasts, the method comprising isolating tumor cells from a subject, contacting the tumor cells with the expansion medium of any of claims 7-12. The cell culture media of the present disclosure can be used to expand tumor cells to use to create tumor-based organoid models for research purposes (e.g., to understand cancer pathology or to test the efficacy of therapeutic agents).

Lung tumor cells can be isolated from a subject suffering from a lung cancer. The tumor cells isolated can be a primary lung tumor or a secondary lung tumor (e.g., a cancer that starts in another tissue and metastasizes to the lungs). Examples of lung tumor cells include but are not limited to small cell lung cancer cells or non-small cell lung cancer cells, including but not limited to, small cell carcinoma, combined small cell carcinoma, adenocarcinoma, squamous cell carcinoma, large cell carcinoma, pancoast tumor cells, neuroendocrine tumor, or lung carcinoid tumor cells. Established lung cancer cell lines can also be used with the culture medium of the present disclosure. Lung cancer cell lines that can be used with cell media of the present disclosure can be found on the ATCC website. Examples of lung cancer cell lines include but are not limited to, EML4-ALK Fusion-A549 Isogenic cell line, NCI-H838[H838], HCC827, SK-LU-1, HCC2935, HCC4006, NCI-H1819 [H1819], NCI-H676B [H676B], Hs 618.T, HBE4-E6/E7 [NBE4-E6/E7], NCI-H1666 [H1666, H1666], NCI-H23 [H23], NCI-H1435 [H1435], NCI-H1563 [H1563], 703D4, and NCI-H1688 [H1688], NCI-H187 [H187], NCI-H661 [H661], NCI-H460 [H460], NCI-H1299, NCI-H1155 [H1551], DMS 114, NCI-H69 [H69], DMS 79, DMS 53, SW 1271 [SW1271, SW1271], SHP-77, NCI-H209 [H209], NCI-H146 [H146], NCI-H345 [H345], NCI-H[341 [H1341], DMS 153, NCI-H82 [H82], NCI-H1048 [H1048], NCI-H128 [H128], NCI-H446 [H446], NCI-H128 [H281], NCI-H510A [H510A], NCI-H510], H69AR, HLF-a, Hs 913T, GCT [Giant Cell Tumor], SW 900 [SW-900, SW900], LL/2 (LLC1), HBE135-E6E7, Tera-2, NCI-H292 [H292], sNF02.2, NCI-H1703 [H1703], NCI-H2172 [H2172], NCI-H2444 [H2444], NCI-H2110 [H2110], NCI-H2135 [H2135], NCI-H2347 [H2347], NCI-H810 [H810], NCI-H1993 [H1993], and NCI-H1792 [H1792].

Another aspect of the present disclosure provides a method of culturing alveolospheres infected with a pathogen, the method comprising consisting of, or consisting essentially of: culturing lung cells with the a culture medium of the present disclosure and inoculating the lung cells with a pathogen in an amount effective to infect the lung cells.

Yet another aspect of the present disclosure provides a method for identifying an agent capable of treating or preventing a pathogen infections in an organoid culture, the method comprising, consisting of, or consisting essentially of: i) culturing the cells in a medium of the present disclosure; ii) inoculating the cells with a pathogen in an amount effective to infect the cells; iii) contacting the cells with an agent; and iv) determining whether the agent causes a reduction in the amount of the pathogen in the cells relative to a cell that has not been treated with the agent.

In some embodiments, the cells or organoid culture is contacted with an agent before the cells are inoculated with a pathogen. Contacting cells with an agent before infection with a pathogen can determine whether the agent is capable of acting as a prophylactic (e.g., able to prevent or reduce the severity of infection with a pathogen).

In other embodiments, the cells or organoid culture is contacted with an agent after the cells are inoculated with a pathogen. Contacting cells with an agent after infection with a pathogen can determine whether the agent is capable of treating a pathogen infection.

In some embodiments, a reduction in the amount of the pathogen in the cells relative to a control cell that has not been treated with the agent can be a reduction of at least about 10%, of at least about 15%, of at least about 20%, of at least about 25%, of at least about 30%, of at least about 35%, of at least about 40%, of at least about 45%, of at least about 50%, of at least about 55%, of at least about 60%, of at least about 65%, of at least about 70%, of at least about 75%, of at least about 80%, of at least about 85%, of at least about 90%, of at least about 95%, or up to and including a 100% reduction, or at least about a 2-fold, or at least about a 3-fold, or at least about a 4-fold, or at least about a 5-fold, at least about a 6-fold, or at least about a 7-fold, or at least about a 8-fold, at least about a 9-fold, or at least about a 10-fold reduction, or any reduction of 10-fold or greater, as compared to a control cell or reference level.

As used herein, the terms “infect” or “infection” refers to affecting a person, organoid, or cell with a disease-causing pathogen.

A pathogen can be a bacterium, virus, or fungus.

In some embodiments, the pathogen is a bacterium, virus, or fungus that infects the lungs of humans or any animal with lungs.

Bacteria that can infect lungs include, but are not limited to Bordetella pertussis, Streptococcus pneumonia, Haemophilus influenza, Staphylococcus aureus, Moraxella catarrhalis, Streptococcus pyogenes, Pseudomonas aeruginosa Neisseria meningitidis, or Klebsiella pneumoniae.

Viruses that can infect lungs include, but are not limited to, 229E (alpha coronavirus), NL63 (alpha coronavirus), OC43 (beta coronavirus), HKU1 (beta coronavirus), MERS-CoV (the beta coronavirus that causes Middle East Respiratory Syndrome, or MERS), SARS-CoV (the beta coronavirus that causes severe acute respiratory syndrome, or SARS), or SARS-CoV-2 (the novel coronavirus that causes coronavirus disease 2019, or COVID-19), an influenza-A virus (e.g., H1N1, H7N9, low pathogenic avian flu, high pathogenic avian flu, or H5N1), an influenza-B virus, respiratory syncytial virus (RSV), or an enterovirus (e.g. enterovirus 71). In some embodiments, the virus is SARS-CoV-2.

Funguses that can infect lungs include, but are not limited to, Aspergillosis.

In some embodiments, the cells that can be infected with a pathogen are tracheal basal cells, bronchiolar secretory cells, club variant cells, alveolar epithelial progenitor cells, clara variant cells, distal lung progenitors, p63+ Krt5− airway cells, lineage negative epithelial progenitors, bronchioalveolar stem cells, Sox9+p63+ cells, neuroendocrine progenitor cells, distal airway stem cells, submucosal gland duct cell, induced pluripotent stem cell-derived lung stem cells, or alveolar type 2 epithelial. In some embodiments, the cells that can be infected with a pathogen are alveolar type 2 epithelial cells (AECs or AT2s).

In some embodiments, the culture medium used with the above method is an expansion medium of the present disclosure, a maintenance medium of the present disclosure, or a differentiation medium of the present disclosure.

An “agent” as used herein refers to a small molecule, protein, peptide, gene, compound or other pharmaceutically active ingredient that can be used for the treatment, prevention, or mitigation of a disease.

Another aspect of the present disclosure provides a method of reducing the viral titers in alveolospheres infected with SARS-CoV-2, the method comprising, consisting of, or consisting essentially of contacting alveolospheres with an agent before the alveolospheres are exposed to SARS-CoV-2, wherein the alvcolospheres exhibit reduced viral titers relative to alveolospheres that have not been contacted with the agent.

In some embodiments of the above methods, the agent is an interferon. An interferon is a group of signaling proteins made and released by host cells in response to the presence of several viruses. An interferon can be a Type I, Type II, or Type III interferon. Examples of interferons include, but are not limited to, INF-α, INF-β, INF-ε, INF-k, INF-w, INF-γ, IL10R2, and INFR1. In some embodiments, the interferon is IFNα and IFNγ.

Kits

Another aspect of the present disclosure provides a kit comprising, consisting of, or consisting essentially of a chemically defined and stroma-free organoid culture system for the culturing, expansion, maintenance and/or differentiation of alveolar epithelial cells, the kit comprising, consisting of, or consisting essentially of a medium of the present disclosure and instructions for use

Another aspect of the present disclosure provides a kit comprising a chemically defined and stroma-free organoid culture system for determining agents to treat or prevent bacterial, viral and fungal infections in organoid cultures, the kit comprising, consisting of, or consisting essentially of a medium of the present disclosure and instructions for use.

Another aspect of the present disclosure provides a kit comprising a chemically defined and stroma-free organoid culture system for determining agents to treat or prevent bacterial, viral and fungal infections in organoid cultures or their derivatives ex vivo and in vivo, the kit comprising, consisting of, or consisting essentially of a medium of the present disclosure and instructions for use.

The following Examples are provided by way of illustration and not by way of limitation.

### EXAMPLES

Materials and Methods

Mice

Sftp^{ctm1(cre/ERT2)Blh}(Sftpc-CreER), Rosa26R-CAG-lsl-tdTomato were maintained on a C57BL/6 background. NU/J (Nude), B61.129(Cg)-Igs2^{tm1.1(CAG-cas9*)Mmef}J (H11-Cas9), B6.129S4-Krastm4Tyj/J (Kras-lsl-G12D) were from the Jackson Laboratory. Ctgf-GFP was kindly gifted from the University of California, Los Angeles. Sftpc-GFP mice were described previously (Blanpain et al., 2014, Science 344, 1242281). For lineage tracing, mice were given 0.2 mg/g Tamoxifen (Sigma-Aldrich, St. Louis, Mo.) via oral gavage. For bleomycin injury, 2.5 U/kg bleomycin was administered intranasally 2 weeks after final dose of Tamoxifen and mice were monitored daily. Animal experiments were approved by the Duke University Institutional Animal care and Use Committee.

Mouse Lung Tissue Dissociation and FACS Sorting

Lune dissociation and FACS were performed as described previously (Chung et al., 2018, Development, 145(9):1-10). Briefly, lungs were intratracheally inflated with 1 ml of enzyme solution containing Dispase (5 U/ml), DNase 1 (0.331 U/ml) and Collagenase type I (450 U/ml) in DMEM/F12. Separated lung lobes were diced and incubated with 3 ml enzyme solution for 30 min at 37° C. with rotation. The reaction was quenched with an equal amount of DMEM/F12+10% FBS medium and filtered through a 100 μm strainer. The cell pellet was resuspended in red blood cell lysis buffer (100 μM EDTA, 10 mM KHCO3, 155 mM NH4Cl) for 5 min, washed with DMEM/F12 containing 10% FBS and filtered through a 40 μm strainer. Total cells were centrifuged at 450 g for 5 min at 4° C. and the cell pellet was processed for AT2 isolation by FACS.

Human Lung Tissue Dissociation

Human lung dissociation was as described previously (Zacharias et al., 2018, Nature 555, 251-255). Briefly, pleura was removed and remaining human lung tissue (approximately 28) washed with PBS containing 1% Antibiotic-Antimycotic and cut into small pieces. Visible small airways and blood vessels were carefully removed to avoid clogging. Then samples were digested with 30 ml of enzyme mixture (Collagenase type 1: 1.68 mg/ml, Dispase: 5 U/ml, DNase: 10 U/ml) at 37° C. for 1 h with rotation. The cells were filtered through a 100 μm strainer and rinsed with 15 ml DMEM/F12+10% FBS medium through the strainer. The supernatant was removed after centrifugation at 450 g for 10 min and the cell pellet was resuspended in red blood cell lysis buffer for 10 min, washed with DMEM/F12 containing 10% FBS and filtered through a 40 μm strainer. Total cells were centrifuged at 450 g for 5 min at 4° C. and the cell pellet was processed for AT2 isolation.

Isolation of Human and Mouse AT2 Cells

AT2 cells were isolated by Magnetic-activated cell sorting (MACS) or Fluorescence-activated cell sorting (FACS) based protocols. For mouse AT2 isolation the total lung cell pellet was resuspended in MACS buffer (1×PBS, pH 7.2, 1% BSA, and 2 mM EDTA). CD31/CD45 positive cells were depleted using MACS beads according to the manufacturer's instructions. After CD31/CD45 depletion AT2 cells were sorted based on TdTomato reporter and for AT2 cells without a reporter, cells were stained using the following antibodies: EpCAM/CD326, PDGFRα/CD140a and Lysotracker as described previously (Katsura et al., 2019, Stem Cell Reports, 12(4):657-666). For isolation of human AT2 cells, approximately 2-10 million total lung cells were resuspended in MACS buffer and incubated with Human TruStain FcX for 15 min at 4° C. followed by HTII-280 (1:60 dilution) antibody for 1 h at 4° C. The cells were washed twice with MACS buffer and then incubated with anti-mouse IgM microbeads for 15 min at 4° C. The cells were loaded into the LS column and labeled cells collected magnetically. For FACS based purification of human AT2 cells, the total lung cell pellet was resuspended in MACS buffer. Cells were positively selected for the EpCAM population using CD326 (EpCAM) microbeads according to the manufacturer's instructions. CD326 selected cells were stained with HTII-280 and LysoTracker at 37° C. for 25 min followed by secondary Alexa anti-mouse IgM-488 for 10 min at 37° C. Sorting was performed using a FACS Vantage SE and SONY S1800S.

Alveolosphere (Organoid) Culture

Mouse conventional Alveolosphere culture (using MTEC medium) was performed as described previously (Barkauskas et al., 2013, J. Clin. Invest. 123, 3025-3036). Briefly, FACS sorted lineage labeled AT2 (1-3×10³) cells from Sftpc-CreER: R26R-lsl-ldTomato mice and PDGFRα (5×10⁴) cells were resuspended in MTEC/Plus or serum free medium and mixed with an equal volume of growth factor-reduced Matrigel (BD Biosciences, San Jose, Calif., #354230).

For feeder free culture, AT2s (1-3×10³) were resuspended in serum free medium and mixed with an equal amount of Matrigel. For Transwell culture, 100 μl of medium/Matrigel mixture was seeded in 24-well 0.4 μm Transwell insert (Falcon). For drop culture, 3 drops of 50 μl of cells-medium/Matrigel mixture were plated in each well of a 6-well plate. The medium was changed every other day.

Serum free medium contained 10 μM SB431542 (Abcam, Cambridge, UK), 3 μM CHIR99021 (Tocris, Bristol, UK), 1 μM BIRB796 (Tocris, Bristol, UK), 5 μg/ml Heparin (Sigma-Aldrich, St. Louis, Mo.), 50 ng/ml human EGF (Gibco), 10 ng/ml mouse FGF10 (R&D systems. Minneapolis, Minn.), 10 μM Y27632 (Selleckchem, Houston, Tex.), Insulin-Transferrin-Selenium (Thermo, Waltham, Mass.), 1% Glutamax (Thermo, Waltham, Mass.), 2% B27 (Thermo, Waltham, Mass.), 1% N2 (Thermo, Waltham, Mass.), 15 mM HEPES (Thermo, Waltham, Mass.), 1.25 mM N-acetylcysteine (Sigma-Aldrich, St. Louis, Mo.) and 1% Anti-Anti (Thermo, Waltham, Mass.) in Advanced DMEM/F12 (Thermo, Waltham, Mass.). For Alveo-Expansion medium, 10 ng/ml mouse IL-1b (BioLegend, San Diego, Calif.), 10 ng/ml mouse TNFa (BioLegend, San Diego, Calif.) were added into serum free medium. For Alveo-Maintenance medium, 10 ng/ml mouse Noggin (Peprotech, Rocky Hill, N.J.) and 1 μM DMH-1 (Tocris, Bristol, UK.) were added into Alveo-Expansion medium. Alveo-Differentiation medium contained ITS, Glutamax, 5 μg/ml Heparin, 5 ng/ml human EGF, 1 μg/ml mouse FGF10, 10% fetal bovine serum and 1% Anti-Anti in Advanced DMEM/F12.

For detailed SFFF and AMM media composition see Table 1.

TABLE 1
Media composition (SFFF, AMM, and ADM) for human
AT2 cells self-renewal or differentiation.

| | | SFFF | | AMM | | ADM | | Treatment |
|---|---|---|---|---|---|---|---|---|
| | Component | concentration | | concentration | | concentration | | period |
| Base medium | Advanced DMEM/F12 | | | | | | | |
| Compounds | SB431542 | 10 | μM | 10 | μM | — | | all time |
| | CHIR99021 | 3 | μM | 3 | μM | — | | all time |
| | BIRB796 | 1 | μM | 1 | μM | — | | all time |
| | DMH-1 | — | | 1 | μM | — | | all time |
| | Y-27632 | 10 | μM | 10 | μM | — | | 0 d-4 d |
| Recombinant | Human EGF | 50 | ng/ml | 50 | ng/ml | 5 | ng/ml | all time |
| proteins | Mouse FGF10 | 10 | ng/ml | 10 | ng/ml | 1 | ng/ml | all time |
| | Mouse IL-1β | 10 | ng/ml | 10 | ng/ml | — | | First 4 days |
| | Mouse Noggin | — | | 10 | ng/ml | — | | all time |
| Supplements | Herapin | 5 | μg/ml | 5 | μg/ml | 5 | μg/ml | all time |
| | B-27 | 1X | | 1X | | 1X | | all time |
| | supplement | | | | | | | |
| | Antibiotic- | 1X | | 1X | | 1X | | all time |
| | Antimycotic | | | | | | | |
| | HEPES | 15 | mM | 15 | mM | — | | all time |
| | GlutaMAX | 1X | | 1X | | 1X | | all time |
| | N-Acetyl- | 1.25 | mM | 1.25 | mM | 1.25 | mM | all time |
| | L-Cysteine | | | | | | | |

| | | | | |
|---|---|---|---|---|
| FBS | — | — | 10% | all time |

For human alveolosphere culture, HTII-280⁺ human AT2s (1-3×10³) were resuspended in scrum free medium and mixed with an equal amount of Matrigel and plated in 6 well plates. For detailed mouse and human serum-free, feeder-free (SFFF) media composition, see Table 1 and Table 2.

TABLE 2
Media composition (SFFF and ADM) for human
AT2 cells self-renewal or differentiation.

| | | Concentration | | Concentration | | Treatment |
|---|---|---|---|---|---|---|
| | Component | SFFF | | ADM | | period |
| Base medium | Advanced DMEM/F12 | | | | | |
| Compounds | SB431542 | 10 | μM | — | | all time |
| | CHIR99021 | 3 | μM | — | | all time |
| | BIRB796 | 1 | μM | — | | all time |
| | Y-27632 | 10 | μM | — | | 0 d-4 d |
| Recombinant | Human EGF | 50 | ng/ml | 5 | ng/ml | all time |
| proteins | Human FGF10 | 10 | ng/ml | 1 | ng/ml | all time |
| Supplements | Heparin | 5 | μg/ml | 5 | μg/ml | all time |
| | B-27 | 1X | | 1X | | all time |
| | supplement | | | | | |
| | Antibiotic- | 1X | | 1X | | all time |
| | Antimycotic | | | | | |
| | HEPES | 15 | mM | — | | all time |
| | GlutaMAX | 1X | | 1X | | all time |
| | N-Acetyl- | 1.25 | mM | 1.25 | mM | all time |
| | L-Cysteine | | | | | |

| | | | |
|---|---|---|---|
| Human scrum | — | 10% | all time |

Alveolosphere Passaging

Mouse alveolosphere passaging experiment was performed in AMM medium, composition as described above. Briefly, FACS sorted mouse AT2 cells (2×10³) were resuspended in AMM medium and mixed with an equal volume of Matrigel. 3 drops of 50 μl of cells-medium/Matrigel mixture were plated in each well of a 6-well plate for each biological replicate (n=3). For every passage mouse IL-1β (10 ng/ml) was added for the first 4 days and subsequently, the media was replaced with AMM without IL-1β. The medium was changed every three days. Mouse alveolosphere were passaged every 10 days. For human alveolosphere passages, AT2 cells (3×10³) were resuspended in SFFF medium and mixed with an equal volume of Matrigel, 3 drops of 50 μl of cells-medium/Matrigel mixture were plated in each well of a 6-well plate for each donor (n=3). Alveolospheres were passaged every 10-14 days.

AT2 Differentiation

For detailed mouse and human AT2-Differentiation medium (ADM) composition see table. For differentiation, mouse alveolospheres were cultured in AMM medium for 10 days were switched to AT2-differentiation medium followed by culture for an additional 7 days, except where stated otherwise. For differentiation, human alveolospheres cultured in SFFF media for 10 days were switched to ADM and cultured for an additional 12-15 days, except where stated otherwise. The medium was changed every three days. Human AT2-Differentiation medium contains human serum instead of FBS. The differentiation medium can also comprise IL-6 (20 ng/mL) instead of serum.

Alveolosphere Infection Experiment for Bulk RNAseq and qPCR Studies

To infect alveolosphere cultures, cells were washed with 1 ml PBS then virus was added to cells at a MOI of 1. Virus and cells were incubated for 3.5 hours at 37° C. after which virus was removed and cell culture media was added. Infection proceeded for 48 or 120 hours and then alveolospheres were washed with PBS, dissociated as described above. Finally, alveolosphere derived cells were stored in Trizol and stored at −80° C.

Infection of AT2 Alveolospheres with SARS-CoV-2

Human alveolosphere cultures were briefly washed twice with 500 μl 1×PBS. SARS-CoV-2-GFP (icSARS-CoV-2-GFP virus was described previously (Hou et al., 2020). Briefly, seven cDNA fragments covering the entire SARS-CoV-2 WA1 genome were amplified by RT-PCR using PrimeSTAR GXL HiFi DNA polymerase. Junctions between each fragment contain non-palindromic sites BsaI (GGTCTCN) or BsmBI (CGTCTCN) each with unique four-nucleotide cohesive ends. Fragment E and F contain two BsmBiI sites at both termini, while other fragments harbor BsaI sites at the junction. Each fragment was cloned into high-copy vector pUC57 and verified by Sanger sequencing. A silent mutation T15102A was introduced into a conserved region in nsp12 in plasmid D as a genetic marker. GFP was inserted by replacing the ORF7 gene. Cultures were then inoculated with 200 μl of 1×10⁷ PFU/ml of icSARS-CoV-2-GFP virus (Hou et al., 2020) or 200 μl of 1×PBS for mock cultures. Alveolospheres were allowed to incubate at 37° C. supplemented with 5% CO2 for 2 h. Following incubation, the inoculum was removed, and alveolosphere cultures were washed three times with 500 μl 1×PBS. 1 mL of SFFF media was added to each culture. Alveolospheres were incubated at 37° C. for 72 h, with samples taken every 24 h during infection. To sample, 100 μl of media was removed. Equal volumes of fresh media were then added to the cultures to replace the sampled volume. Viral titers were ultimately determined after 72 h by plaque assay on Vero E6 cells (USAMRIID). Viral plaques were visualized by neutral red staining after 3 days (Hou et al., 2020). For histological analysis alveolospheres were fixed for 7 days in 10% formalin solution followed by 3 washes in PBS.

Interferon Treatment

For interferon and cytokine treatment experiment, Human AT2 cells (2.5×10⁴) from P2 or P3 passage were cultured on the surface of matrigel. Prior to the plating of cells 12 well plates were precoated with matrigel (1:1 matrigel and SFFFM mix) for 30 min. AT2 cells were grown in SFFFM without IL-1β for 7 to 10 days to allow the formation of alveolospheres. Alveolospheres were treated with 20 ng/ml interferons (IFNα, IFNβ, IFNγ) for 12 h or 72 h for RNA isolation and quantitative PCR. For histological analysis, Alveolospheres were treated with indicated interferons for 72 h. Human alveolosphere cultures were pretreated with 10 ng TFNα or 10 ng IFNγ for 18 h prior to virus infection. For IFN inhibition studies, alveolospheres were treated with 1 μM Ruxolitinib throughout the culture time.

RNA Isolation and qRT-PCR

For RNA isolation, Alveolospheres were dissociated into single-cell suspension using TrypLE™ Select Enzyme at 37° C. for 10 min. The cell pellet was resuspended in 300 μl of TRIzol™ LS Reagent Total RNA was extracted using the Direct-zol RNA MicroPrep kit according to the manufacturer's instructions with DNase 1 treatment. Reverse transcription was performed from 600 ng of isolated total RNA of each sample using SuperScript III with random hexamer or negative-strand specific primer. Quantitative RTPCR assays were performed using StepOnePlus system (Applied Biosystems) with PowerUp™ SYBR™ Green Master Mix. The relative quantities of mRNA for all target genes were determined using the standard curve method. Target-gene transcripts in each sample were normalized to Glyceraldehyde 3-phosphate dehydrogenase (GAPDH). Primers used are listed in Table 3.

TABLE 3
Primers

| Species | Gene | Sequence |
|---|---|---|
| Human | ACE2_Forward | ATCAGAGATCGGAAGAAGAAAA (SEQ ID |
| | | NO: 02) |
| Human | ACE2_Reverse | TTGCTAATATCGATGGAGGCA (SEQ ID NO: 03) |
| Human | TMPRSS2_Forward | CCGAGGAGAAAGGGAAGACC (SEQ ID NO: 04) |
| Human | TMPRSS2_Reverse | TCACCCTGGCAAGAATCGAC (SEQ ID NO: 05) |
| Human | SFTPB_Forward | CCATGATTCCCAAGGGTGCG (SEQ ID NO: 06) |
| Human | SFTPB_Reverse | CAGCCATTCTCCTGTCGGC (SEQ ID NO: 07) |
| Human | SFTPC_Forward | TCCAGAGAGCATCCCCAGTC (SEQ ID NO: 08) |
| Human | SFTPC_Reverse | GGCTTCCACTGACCCTGC (SEQ ID NO: 09) |
| Human | ABCA3_Forward | AGATGTAGCGGACGAGAGGA (SEQ ID NO: 10) |
| Human | ABCA3_Reverse | GCTGCTCGTACACCTTGGAG (SEQ ID NO: 11) |
| Human | LAMP3_Forward | AAGATGACCACTTTGGAAATGTG (SEQ ID |
| | | NO: 12) |
| Human | LAMP3_Reverse | GATGGCCCCAATCACAGGAA (SEQ ID NO: 13) |
| Human | IFNA7_Forward | GGCCCGGTCCTTTTCTTTAC (SEQ ID NO: 14) |
| Human | IFNA7_Reverse | ACTCCTCCTCTGGGAATCTGAA (SEQ ID NO: 15) |
| Human | IFNB1_Forward | ACGCCGCATTGACCATCTA (SEQ ID NO: 16) |
| Human | IFNB1_Reverse | TGGCCTTCAGGTAATGCAGA (SEQ ID NO: 17) |
| Human | IFNL1_Forward | GGTGACTTTGGTGCTAGGC (SEQ ID NO: 18) |
| Human | IFNL1_Reverse | AGTGACTCTTCCAAGGCG (SEQ ID NO: 19) |
| Human | IFIT1_Forward | ATTTACAGCAACCATGAGTACAAA (SEQ ID |
| | | NO: 20) |
| Human | IFIT1_Reverse | TCCCACACTGTATTTGGTGTC (SEQ ID NO: 21) |
| Human | IFIT2_Forward | TGCAACCATGAGTGAGAACA (SEQ ID NO: 22) |
| Human | IFIT2_Reverse | GATAGGCCAGTAGGTTGCACA (SEQ ID NO: 23) |
| Human | IFIT3_Forward | CAGAACTGCAGGGAAACAGC (SEQ ID NO: 24) |
| Human | IFIT3_Reverse | GGAAGGATTTTCTCCAGGG (SEQ ID NO: 25) |
| Human | CXCL10_Forward | AAGTGGCATTCAAGGAGTACC (SEQ ID NO: 26) |
| Human | CXCL10_Reverse | ACGTGGACAAAATTGGCTTGC (SEQ ID NO: 27) |
| Human | IL6_Forward | CTCCTTCTCCACAAGCGCC (SEQ ID NO: 28) |
| Human | IL6_Reverse | GAAGGCAGCAGGCAACAC (SEQ ID NO: 29) |
| Human | IL1A_Forward | TGAGTCAGCAAAGAAGTCAAG (SEQ ID NO: 30) |
| Human | IL1A_Reverse | GGAGTGGGCCATAGCTTACA (SEQ ID NO: 31) |
| Human | IL1B_Forward | TTCGAGGCACAAGGCACAA (SEQ ID NO: 32) |
| Human | IL1B_Reverse | TGGCTGCTTCAGACACTTGAG (SEQ ID NO: 33) |
| Human | GAPDH_Forward | TCGGAGTCAACGGATTTGG (SEQ ID NO: 34) |
| Human | GAPDH_Reverse | TTCCCGTTCTCAGCCTTGAC (SEQ ID NO: 35) |
| Mouse | Sftpc_Forward | ACAATCACCACCACAACGAG (SEQ ID NO: 36) |
| Mouse | Sftpc_Reverse | AGCAAAGAGGTCCTGATGGA (SEQ ID NO: 37) |
| Mouse | Abca3_Forward | CCGCCTCAGTTGTCAGCTTC (SEQ ID NO: 38) |
| Mouse | Abca3_Reverse | ACATCACAGTGGAGGGATAGTG (SEQ ID |
| | | NO: 39) |
| Mouse | Lamp3_Forward | GCTTGGTGTTCCTTGGTGTTC (SEQ ID NO: 40) |
| Mouse | Lamp3_Reverse | CCACTGTTGTGTGCTTGAGTC (SEQ ID NO: 41) |
| Mouse | Gapdh_Forward | TTGAGGTCAATGAAGGGGTC (SEQ ID NO: 42) |
| Mouse | Gapdh_Reverse | TCGTCCCGTAGACAAAATGG (SEQ ID NO: 43) |
| SARS- | N3_Forward | GGGAGCCTTGAATACACCAAAA (SEQ ID |
| CoV-2 | | NO: 44) |
| SARS- | N3_Reverse | TGTAGCACGATTGCAGCATTG (SEQ ID NO: 45) |
| CoV-2 | | |
| SARS- | Negative strand- | ACTGGAACACTAAACATAGCAGTGGTGTTA |
| CoV-2 | specific RT primer | (SEQ ID NO: 46) |
| SARS- | genome_1202- | AACCAAATGTGCCTTTCAACTC (SEQ ID |
| CoV-2 | 1363_Forward | NO: 47) |
| SARS- | genome_1202- | AACAACAGCATTTTGGGGTAAG (SEQ ID |
| CoV-2 | 1363_Reverse | NO: 48) |
| SARS- | genome_848- | GGCTACCCTCTTGAGTGCATTA (SEQ ID NO: 49) |
| CoV-2 | 981_Forward | |
| SARS- | genome_848- | GCAATTTCATGCTCATGTTCAC (SEQ ID NO: 50) |
| CoV-2 | 981_Reverse | |

Bulk RNA Sequencing and Differential Gene Expression Analysis

Purified RNA (1 μg) from each sample was enriched for Poly-A RNA using NEBNext Poly(A) mRNA Magnetic Isolation Module (New England BioLabs, Ipswich, Mass., #E7490). Libraries were prepared using NEBNext Ultra 11 RNA Library Prep Kit for Illumina (New England BioLabs, Ipswich, Mass., #E7770). Paired-end sequencing (150 bp for each read) was performed using HiSeq X with at least 15 million reads for each sample. Quality of sequenced reads were assessed using FastQC (www.bioinformatics.babraham.ac.uk/projects/fastqc/). PolyA/T tails were trimmed using Cutadapt (Martin, 2011). Adaptor sequences were trimmed and reads shorter than 24 bp were trimmed using Trimmomatic (Bolger et al., 2014). Reads were mapped to the reference genomes of human (hg38) and SARS-CoV2 (wuhCor1) obtained from UCSC using Hisat2 (Kim et al., 2019) with default setting. Duplicate reads were removed using SAMtools (Li et al., 2009). Fragment numbers were counted using the featureCounts option of SUBREAD (Liao et al., 2014). Normalization and extraction of differentially expressed genes (DEGs) between control and treatments were performed using an R package, DESeq2 (Love et al., 2014).

Tumor Organoid Culture

K-raslsI-G12D; Rosa26R-CAG-lsl-tdTomato mice were induced with tumors using adenovirus carrying Cre recombinase and GFP (SignaGen Laboratories, SL100706). Mice were intranasally infected with approximately 2.5×10⁷ plaque-forming units of virus in 100 μl around 6-8 weeks of age. Lungs were isolated at least 8 months after tumor induction. Visible tumor nodules were manually dissected under a microscope and dissociated as described above. Cells were stained with anti-EPCAM/CD326 antibody and Lysotracker and tumor cells were sorted as tdTomato+, EPCAM+ and Lysotracker+ population by using SONY SH1800S. FACS-sorted cells were resuspended in medium and mixed with equal amount of Matrigel. Three drops containing 2×10³ cells in 50 μl were plated in 6 well plate. Medium were changed every other day.

Grafting of Organoid Derived Cells

Organoids were dissociated into single cells with Accutase (Sigma-Aldrich) followed by 0.25% trypsin-EDTA treatment on day 10-12 and resuspended in serum free medium with 1% Matrigel and 10 mM EDTA. Nude mice were intratracheally injected 80 μl of medium containing 5-7×10⁵ cells 10 days after intranasal administration of bleomycin. Lungs were fixed and analyzed at least 2 months after grafting.

Tissue Preparation and Sectioning

Lungs and alveolospheres from Transwell were fixed with 4% paraformaldehyde (PFA) at 4° C. for 4 h and at room temperature for 30 min, respectively. Organoid cultures from drop were first immersed with 1% low melting agarose (Sigma) and fixed with 4% at room temperature for 30 min. For OCT frozen blocks, samples were washed with PBS and incubated with 30% sucrose at 4° C. overnight. And then samples were incubated with 1:1 mixture of 30% sucrose/OCT for 4 h at 4° C., embedded in OCT and cryosectioned (10 μm). For paraffin blocks, samples were dehydrated, embedded in paraffin and sectioned at 7 μm.

Immunostaining

Paraffin sections were first dewaxed and rehydrated before antigen retrieval. Antigen retrieval was performed by using 10 mM sodium citrate buffer in antigen retrieval system (Electron Microscopy Sciences, Hatfield, Pa.) or water bath (90° C. for 15 min) or 0.05% Trypsin (Sigma-Aldrich. St. Louis, Mo.) treatment for 5 min at room temperature. Sections were washed with PBS, permeabilized and blocked with 3% BSA and 0.1% Triton X-100 in PBS for 30 min at room temperature followed by incubation with primary antibodies at 4° C. overnight. Then sections were washed with 0.05% Tween-20 in PBS (PBST) 3 times, incubated with secondary antibodies in blocking buffer for 1 h at room temperature, washed with PBST 3 times and mounted using Fluor G reagent with DAPI. Primary antibodies were as follows: Prosurfactant protein C (Millipore, Burlington, Mass. ab3786, 1:500), RAGE/AGER (R&D systems, Minneapolis. Minn., MAB1179, 1:250), HOPX (Santa Cruz Biotechnology, Dallas, Tex., sc-30216, 1:250, sc-398703, 1:250), T1a/PODOPLANIN (DSHB, clone 81.1, 1:1000), KRT8 (DSHB, TROMA-1, 1:50), tdTomato (ORIGENE, A138181-200, 1:500), CLDN4 (Invitrogen, Carlsbad, Calif. 36-4800, 1:200), GFP (Novus Biologicals, Littleton, Colo., NB100-1770, 1:500).

For quantifying the stainings on near single cell suspensions, Alveolosphere bubbles were dissociated using TtypLE™ Select Enzyme at 37° C. for 15 min. Matrigel was disrupted by vigorous pipetting. Alveolosphere derived cells were then plated on matrigel precoated (5-10% Matrigel for 30 min) coverslips or chamber slides for 2-3 h. Cells were then fixed in 4% paraformaldehyde.

Electron Microscopy

Organoids were fixed for 3 h in 2.5% glutaraldehyde (Electron Microscopy Sciences, EMS, Hatfield, Pa.) in 0.1M cacodylate buffer pH 7.4 (Electron Microscopy Sciences, EMS, Hatfield, Pa.) at room temperature. The sample was then washed in 0.1M cacodylate three times for 10 min each, post-fixed in 1% Tannic Acid (Sigma) in 0.1M cacodylate buffer for 5 min at room temperature and washed again three times in 0.1M cacodylate buffer. Organoids were post fixed overnight in 1% osmium tetroxide (Electron Microscopy Sciences, EMS) in 0.1M cacodylate buffer in dark at 4° C. The sample was washed three times in 0, IN acetate buffer for 10 min and block stained in 1% Uranyl acetate (Electron Microscopy Sciences, EMS, Hatfield, Pa.) for one hour at room temperature. Next, the sample was dehydrated through acetone on ice: 70%, 80%, 90%, 100% for 10 min each and then incubated with propylene oxide at room temperature for 15 min. The sample was changed into EMbed 812 (EMS), left for 3 hours at room temperature. Changed into fresh Embed 812 and left overnight at room temperature, after which it was embedded in freshly prepared EMbed 812 and polymerized overnight at 60° C. Embedded samples were thin sectioned at 70 nm and grids were stained in 1% aqueous Uranyl Acetate for 5 min at room temperature followed by Lead Citrate for 2.5 min at room temperature. Sections on grids were imaged on FEI Tecnai G2 Twin at magnification of 2200× and 14500×.

Whole Mount Imaging

For whole mount imaging of lungs, lungs were fixed with 4% PEA and cleared by CUBIC-15. Images were obtained by using fluorescence stereoscope (Zeiss Lumar, V12). For organoid, AEC2 cells isolated from Sftpc-CreER; Rosa26R-lsl-tdTomato were grown on 35 mm glass bottom culture dishes in Alveo-Expansion medium and organoids were fixed on day 7 and 10 of culture in 4% PFA for 30 min at room temperature. Then samples were washed four times 30 mini each in PBST (1×PBS+0.1% TritonX-100) blocked in blocking solution (1.5% BSA in 1×PBS+0.3% TritonX-100) for 1 hour at room temperature and incubated with anti-SFTPC (1:500, Millipore, Burlington, Mass.) and anti-AGER (1:500 R&D) in blocking solution overnight at 37° C. Organoids were then washed in PBST (4×30 min), incubated with secondary antibodies in PBST for 1 hour at 37° C. and washed once in PBST+ DAPI for 30 min and twice in PBST for 30 min each at room temperature. Images were captured using Olympus Confocal Microscope FV3000 using a 20× or 40× objective.

Live Imaging

AEC2 cells isolated form Sftpc-GFP mouse were grown on 35 mm glass-bottom culture dishes for 3 days in Alveo-Expansion medium. DIC images were acquired at intervals of 20 min with a microscope (VivaView-Olympus). After 3 days of imaging (day 6 of culture) medium was changed and imaging was started again (day 8 of culture) and continued for additional 2 days.

Plasmid Construction, AAV6 Production and HITI-Based Gene Editing in Organoid

Sftpc-specific gRNA vector was prepared by using AAV:ITR-U6-sgRNA-hSyn-Cre-2AEGFP-KASH-WPRE-shortPA-ITR (Addgene plasmid #60231) as a backbone. First, hSyn-Cre-2A-EGFP-KASH-WPRE cassette was removed by XbaI and RsrlI digestion and EGFP gene flanked by gRNA binding sequence was cloned into the plasmid. Sftpc-specific gRNA was designed close to the end of coding region by using a web tool for selecting target sites for CRISPR/Cas9 “CHOPCHOP” and was inserted into the SapI site at the downstream of U6 promoter. The CRISPR/Cas9 target sequences (20 bp target and 3 bp PAM sequence (underlined) used in this study are GGATGCTAGATATAGTAGAGTGG (SEQ ID NO:01). Small scale AAV production followed the recently published method. In brief, HEK293T cells were plated on a 12 well plate, then transfected with 0.4 μg AAV plasmid, 0.8 μg helper plasmid pAd-DeltaF6, and 0.4 μg serotype 2/6 plasmid per well with PEI Max (Polysciences, Warrington, Pa.; 24765) when cell density reached 60-80% confluency, Twelve hours later, cells were then incubated in glutaminefree DMEM (ThermoFisher, Waltham, Mass.; 11960044) supplemented with 1% Glutamax (ThermoFisher, Waltham, Mass.; 35050061) and 10% FBS for 2 days. The AAV-containing supernatant medium was collected and filtered through a 0.45 μm filter tube and stored at 4° C. until use. For gene editing, AEC2s (EPCAM+ Lysotracker+ cells) were isolated from H11-Cas9 mice. AEC2s (5×10⁴) were resuspended in Alveo-Expansion medium and incubated with 100 μl of AAV-containing supernatant at 37° C. for 1 h with rotation. The cells were washed with PBS, resuspended in Alveo-Expansion medium, mixed with equal amount of Matrigel and plated in 6 well plate. Alveo-Expansion medium was changed every other day. Once the organoids grew, these were dissociated into single cells as described above and GFP+ cells were purified by FACS.

Droplet-Based Single-Cell RNA Sequencing (Drop-Seq)

Organoids embedded in Matrigel were incubated with Accutase at 37° C. for 20 min followed by incubation with 0.25% trypsin-EDTA at 37° C. for 10 min. Trypsin was inactivated using DMEM/F-12 Ham supplemented with 10% FBS then cells were resuspended in PBS supplemented with 0.01% BSA. The cells filtered through 40 μm strainer were utilized at 100 cells/μl for running through microfluidic channels with flows of cells at 3,000 μl/hr, mRNA capture beads at 3,000 μl/hr and droplet-generation oil at 13,000 μl/hr. DNA polymerase for pre-amplification step (1 cycle of 95′C for 3 min, 15-17 cycles of 98° C. for 15 sec, 65° C. for 30 sec, 68° C. for 4 min and 1 cycle of 72′C for 10 min, adopted from 8) was replaced by Terra PCR Direct Polymerase (#639271, Takara). The other processes were performed as described in original Drop-seq protocol9. Libraries were sequenced using HiSeq X with 150-bp paired end sequencing.

Computational Analysis for Drop-Seq

The FASTQ files were processed using dropSeqPipe v0.3 (hoohm.github.io/dropSeqPipe) and mapped on the GRCm38 genome with annotation version 91. Unique molecular identifier (UMI) counts were then further analyzed using an R package Seurat v3.0.6 (Stuart et al., 2019). UMI counts were normalized using SCTransform v0.2 (Hafemeister and Satija, 2019). Principle components which are significant based on Jackstraw plots were used for generating t-SNE plots. After excluding duplets, specific cell clusters were identified based on enrichment for Sftpc, Sftpa1, Sftpa2, Sftpb, Lamp3, Abca3, Hopx, Ager, Akap5, Epcam, Vim, Pdgfra, Ptprc, Pecam1 and Mkt67 in tSNE plot.

Computational Analysis for Single-Cell RNA Sequencing of COVID-19 Patient Lungs

Publicly available single-cell RNA-seq dataset of six severe COVID-19 patient lungs (GSE145926 (Bost et al., 2020, Cell, 181(7):1475-1488)) and control lungs (GSE135893 (Habermann et al., 2019)) were obtained from Gene Expression Omnibus (GEO). EpCAM-positive epithelial cell cluster in the severe COVID-19 patient lungs was further clustered based on LAMP3, ABC43, KRT5, KRT15, DNAH1, FOXJ1, SCGB3A1 and SCGB1A1. AT2 cells that have ≥1 UMI count of LAMP3, NKX2-1 and ABCA3 were utilized for comparison between severe COVID-19 patient lungs and control lungs. UMI counts were normalized and regressed to percentage of mitochondrial genes using SCTransform. Enriched genes in severe COVID-19 patient and control lungs were extracted using FindMarkers and shown in volcano plot drawn by R package Enhanced Volcano v 1.5.4 Genes that have ≥2 log 2 fold change were used as input for Enrichr (Kuleshov et al., 2016) query to get enriched signaling pathways through database—BioPlanet.

Statistics

Sample size was not predetermined. Data are presented as means with standard error (s.e.m) to indicate the variation within each experiment. Statistics analysis was performed in Excel, Prism and R. A two-tailed Student's t-test was used for the comparison between two experimental conditions. For experiments with more than two conditions, statistics significance was calculated by ANOVA followed by the Tukey-HSD method. The Shapiro-Wilk test was used to test whether data are normally distributed and used Wilcoxon rank sum test for the comparison between two conditions that showed non-normal distributions. For more than two conditions, we used Steel-Dwass test.

### Example 1: Establishment of Chemically Defined Conditions for Alveolar Organoid Cultures

Previous studies have demonstrated that the lung resident PDGFRa+ fibroblasts can support the growth of AEC2s when they are co-cultured in MTEC medium, which contains scrum and many unknown components (see methods section for details) (Schwartz et al., 2018, Ann. Am. Thorac. Soc. 15, S192-S197, Barkauskas et al., 2013, J. Clin. Invest. 123, 3025-3036, Frank et al., 2016, Cell Rep. 17, 2312-2325, Katsura et al., 2019, Stem Cell Rep. 12, 657-666, Lee et al., 2014, Cell 156, 440-455, Lee et al., 2013, Am. J. Respir. Cell Mol. Biol. 48, 288-298. Interestingly, AEc2s do not replicate in the absence of PDGFRa+ fibroblasts implying that either paracrine or contact mediated signals that emanate from fibroblasts are essential for the AEC2s propagation.

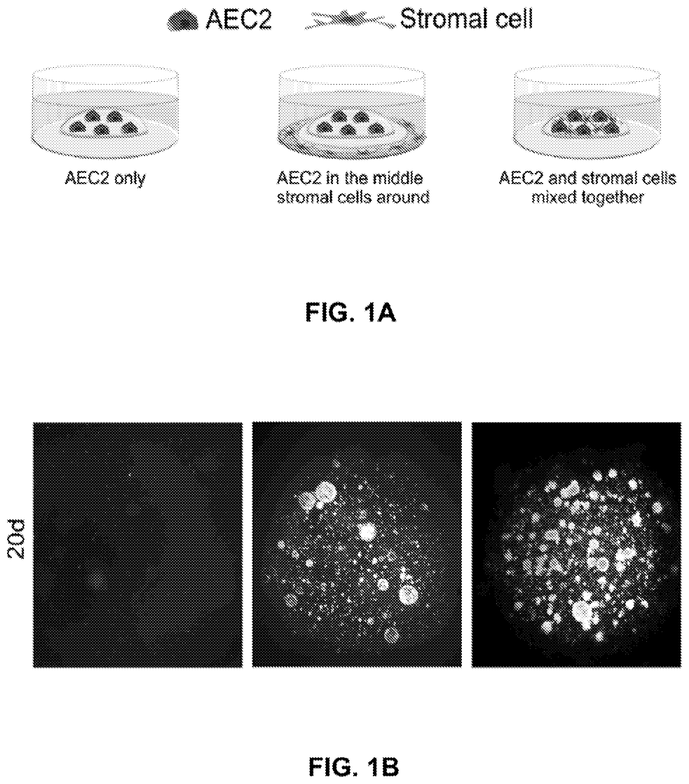

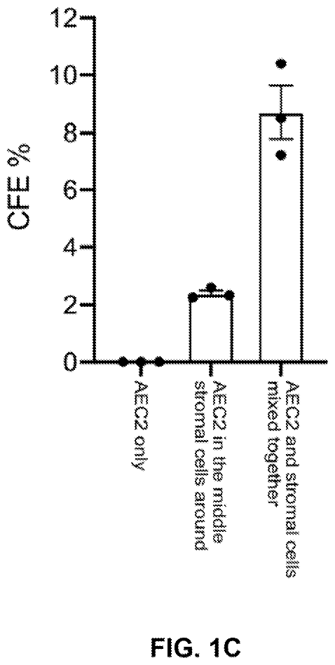

To dissect the nature of communication (i.e., paracrine or contact mediated), AEC2-fibroblast co-culture system was set up in three different modes: i) AEC2 cells only (condition-A); ii) AEC2s and fibroblasts were physically separated (condition—B); and iii) AEC2s mixed with fibroblasts (condition—C). It was found that condition—C yielded the maximal colony forming efficiency (CFE) (8.71%±0.92%) and a moderate to low (2.40%±0.10%) in condition-B and no organoids (0%±0%) were observed in condition—A (FIGS. 1A-1C). These data suggest that contact mediated signaling is not necessary and a short range paracrine signaling is mediating the communication between fibroblasts and AEC2s.

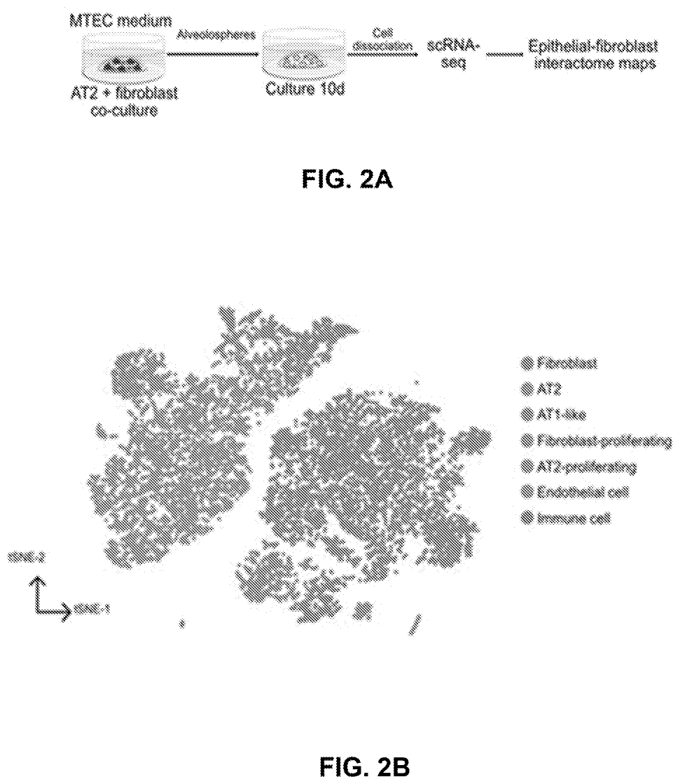

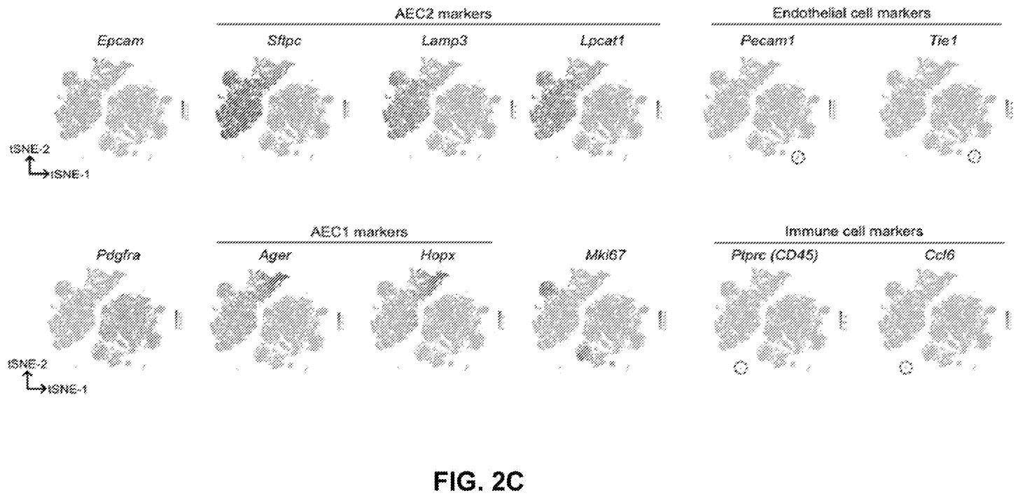

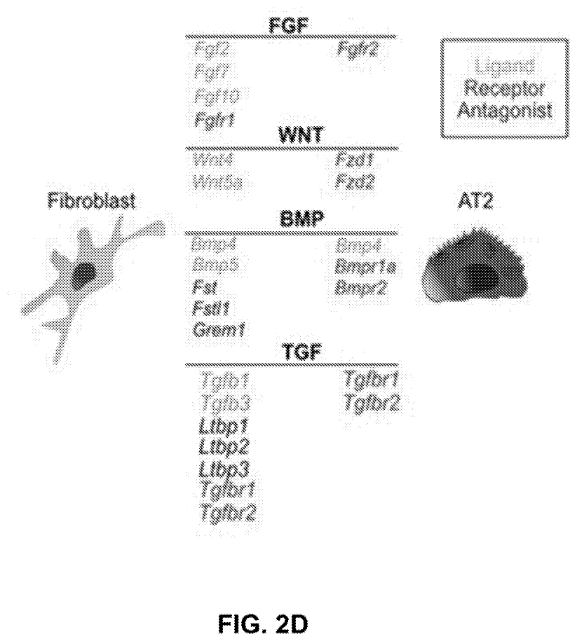

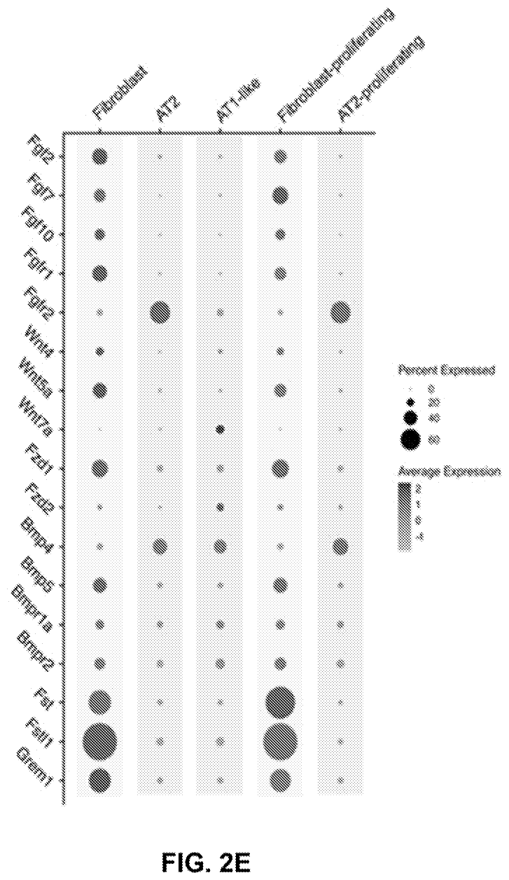

To identify the paracrine signals communicating between these cells, single-cell transcriptome analysis was performed on cells from the above co-culture system. After quality control filtering, k-means clustering was performed and the cells were visualized by stochastic neighbor embedding (t-SNE) and two major clusters consisting of EpCAM+ epithelial cells and Vimentin+/Pdgfra stromal cells were identified. Of note, two small clusters (<10 cells each) consisting of Pecam+ endothelial cells and Ptprc+ immune cells were observed (FIG. 2A, FIG. 2B, and FIG. 2C). Within epithelial cell clusters, three sub-clusters consisting of Sftpc+ AEC2s, Ager+ AEC1s, and Sftpc+/MAi67+ proliferating AEC2s were observed. Of note, Acta2+/Pdgfra+ myofibroblasts within Pdgfra+ cells were found. These data indicate that 3-dimensional organoid cultures resemble cellular diversity and gene expression profiles similar to their in vivo counter parts. scRNA-seq analysis indicated the receptor-ligand interactions in developmental pathways between epithelial and stromal cells in alveolar organoid culture. However, these processes occur spontaneously, presumably mediated by stroma and serum containing culture conditions.

To achieve a more defined culture system, the above scRNA-seq data was mined to find ligand-receptor pairs expressed in epithelial and fibroblasts. Many signaling pathway components that are differentially enriched in AEC2s and fibroblasts were found. Notably, many ligands of wnt (wnt4, wnt5a), BMP (Bmp4, Bmp5), TGFb (Tgfb1, Tgfb3), and FGF (Fgf2, Fgf7, Fgf10) signaling pathways in fibroblasts were found, whereas the corresponding receptors were identified in AEC2s wnt (Fzd1, Fzd2), BMP (Bmpr1a, Bmpr2), TGFb (Tgfbr1, Tgfbr2), and FGF (Fgfr1, Fgfr2) (FIG. 2D and FIG. 2E). Interestingly, it was also found that inhibitors of BMP (Fst, Fstl1, Grem1) and TGFβ (Ltbp1, Ltbp2, Ltbp3) are also enriched in fibroblasts. These data indicate that fibroblasts may dynamically and spatially regulate both proliferation and differentiation of AEC2s.

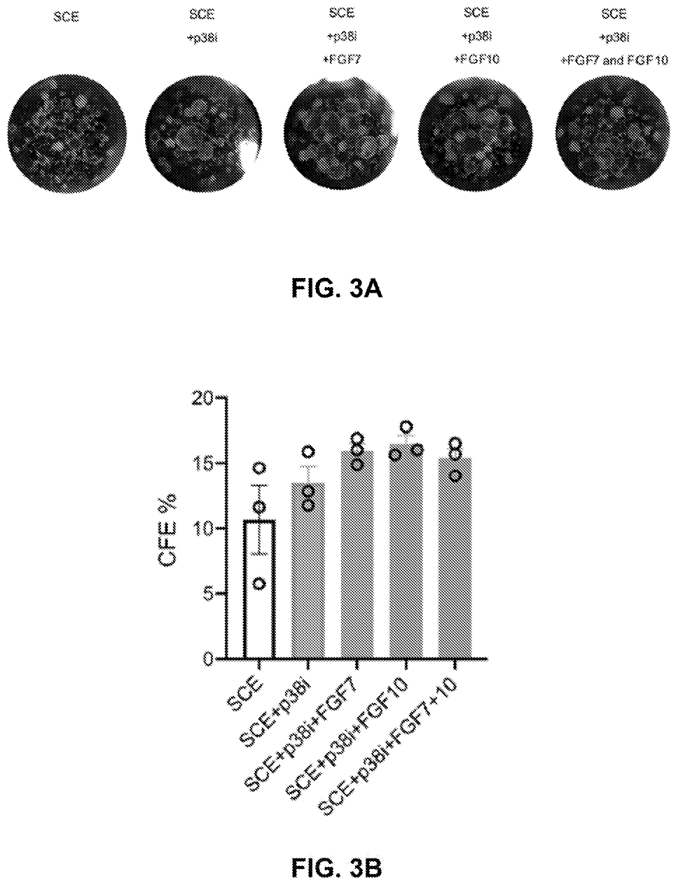

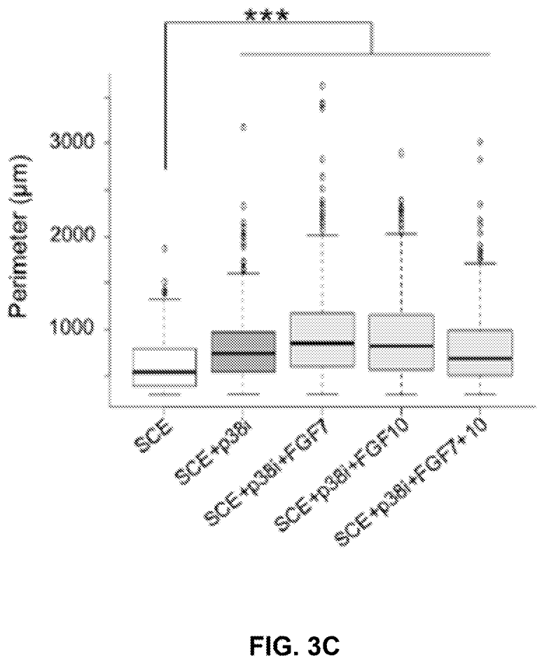

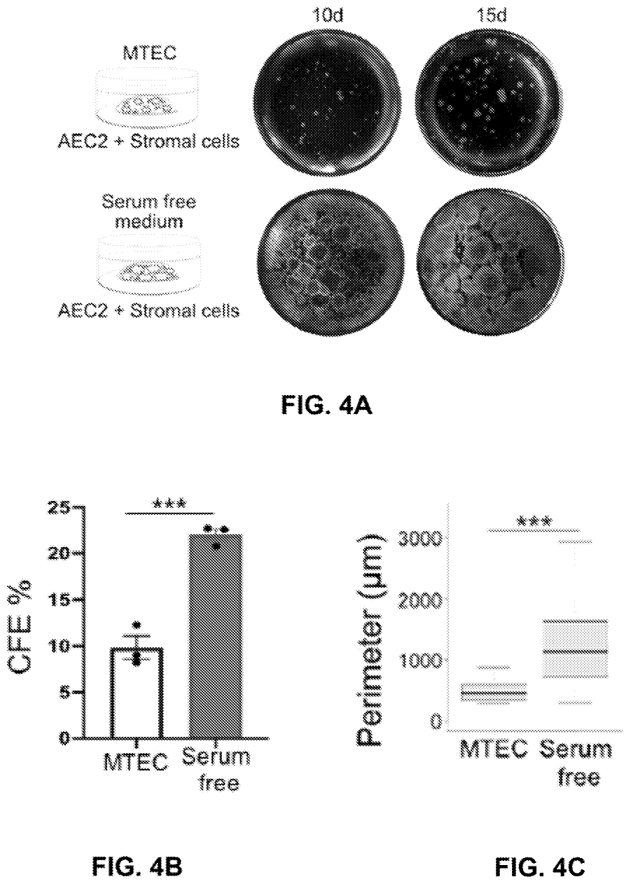

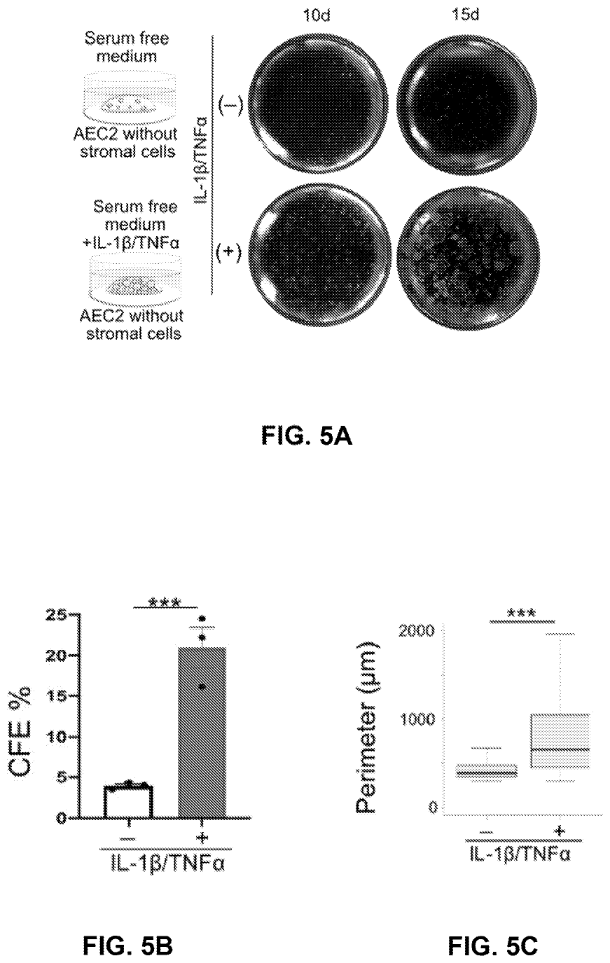

To develop scrum-free and chemically media for AEC2 culture, small molecule modulators or ligands for specific receptors for pathway modulation were used. Previous studies have demonstrated that activation of wnt and EGF pathways and inhibition of TGF pathways is essential for AEC2 replication. In addition, the scRNA-seq guided interactome analysis further supported the requirement for wnt and FGF and inhibition of TGFβ pathways for AEC2 maintenance and replication FIG. 2D and FIG. 2E). Therefore, a base media containing known concentrations of essential nutrients that are critical for the cell growth was formulated and this media was supplemented with CHIR, EGF, and SB431542. This medium was tested in AEC2-fibroblast co-culture system and found that albeit low CFE and colony size. AEC2s can proliferate in this medium without the need for serum and other unknown factors derived from bovine pituitary extract. This media was used as a base media and tested other pathways including p38 kinase inhibition (known to enhance EGF pathway), FGF7, FGF9, and FG10. While a modest effect of p38 inhibition on AEC2 proliferation was observed, both FGF7 and FGF10 alone or in combination gave maximal CFE. There was no additive effect on the CFE (10.7%±2.6% in SCE versus 13.5%±1.2% in SCE+p38i versus 15.9%±0.6% in SCE+p38i+FGF7 versus 16.5%±0.7% in SCE+p38i+FGF10 versus 15.4%±0.7% in in SCE+p38i+FGF7+10 [n=3] on day 15; mean±SEM) or size (629.7±170.7 μm in SCE versus 823.8±228.3 μm in SCE±p38i versus 967.6±304.8 In in SCE+p38i+FGF7 versus 921.1±271.2 μm in SCE+p38i+FGF10 versus 812.3±256.2 μm in SCE+p38i+FGF7+10 [n=3]; mean±SEM) of the organoids when both FGF7 and FGF10 were added to the organoid cultures (FIG. 3A, FIG. 3B, and FIG. 3C). Notably, a significant increase in the CFE (9.8%±0.8% in MTEC [n=3] versus 22.0%±0.5% in serum free [n=3] on day 10; mean±SEM) and colony size (505.0±104.7 μm in MTEC versus 1228.2±363.7 μm in serum free [n=3]; mean±SEM) in the newly formulated medium was found (FIG. 4A, FIG. 4B, and FIG. 4C).

Immunofluorescence analysis for AEC2 (SFTPC) and AEC1 (AGER also known as RAGE) markers revealed that the organoids are composed of both AEC2 and AEC1 (data not shown). Of note, many cells that co-express AEC2 and AEC1 markers were observed.

These data revealed that the new media described in this example can replace serum and bovine pituitary extract that are present in previously used MTC media.

### Example 2: Transient IL1 Treatment Overcomes Fibroblasts Dependency in Organoid Cultures

To test whether the above medium can support AEC2 cell growth without fibroblasts, AEC2 organoid cultures were setup in the absence of fibroblasts. Very small and fewer organoids were observed in these conditions, indicating that AEC2s require additional factors for their growth. Previous studies have demonstrated that IL1β/TNFa mediated NFkB signaling is essential for AEC2 cell replication and regeneration after injury and serve as component of the AEC2 niche (Katsura et al., 2019, Stem Cell Rep. 12, 657-666). Therefore, IL1s and TNFa were added to the above serum-free media and tested whether these conditions can replace fibroblasts in AEC2 organoid cultures. Numerous organoids that were significantly bigger in size compared to controls (no IL1β/TNFa) were observed. Of note, CFE in IL1β treated cultures reached similar efficiency as fibroblast containing conditions. In addition, immunofluorescence analysis suggests that these organoids are composed of both AEC2 and AEC1. Similar organoid size (433.4±77.7 μm without IL1s/TNFa versus 857.2±339.5 μm with IL1β/TNFa [n=3]; mean±SEM) and CFE (4.0%±0.3% without IL1β/TNFa [n=3] versus 21.0%±1.3% with IL1β/TNFa [n=3] on day 15; mean±SEM) was observed in IL1β alone or TNFa alone or in combination, indicating that either IL1s or TNFa is sufficient to replace fibroblasts while maintaining AEC2 self-renewal and differentiation (FIG. 5A, FIG. 5B, and FIG. 5C and data not shown). IL1β/TNFa-mediated NFkB signaling is known to have multifaceted functions to regulate cell proliferation, survival and apoptosis and is associated with early stages of tissue injury repair processes in vivo LaCanna et al., 2019, J. Clin. Invest, 129, 2107-2122: Karin et al., 2009. Cold Spring Harb. Perspect. Biol. 1, a000141, DiDonato et al., 2012, Immunol. Rev. 246, 379-400, Cheng et al, 2007, J. Immunol. Baltim. Md 1950 178, 6504-6513.

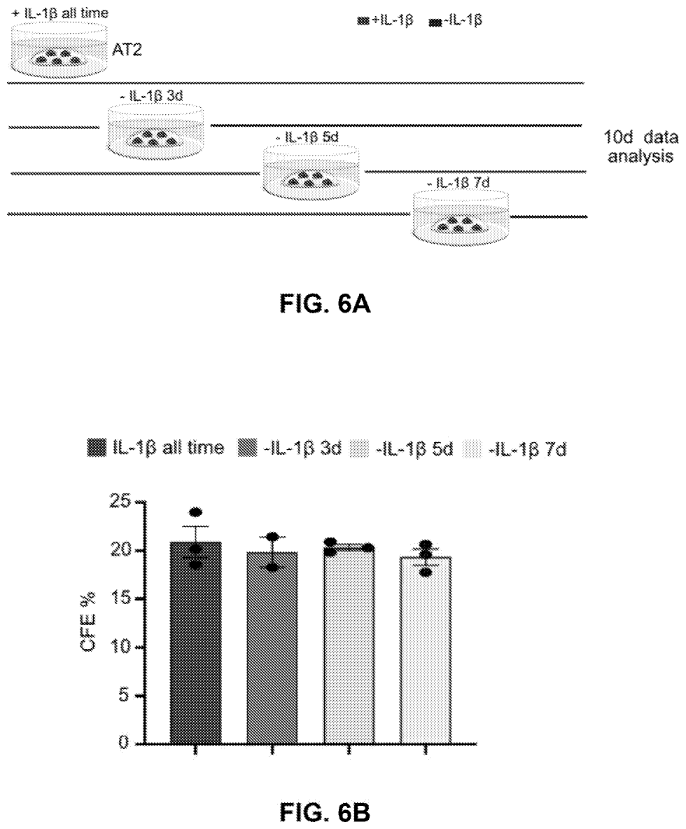

It was therefore asked whether IL1β treatment is necessary in the early stages or throughout the culture period. To test this, IL1β was removed at different day points after the organoid culture setup. No decrease in CFE even when IL1β was removed from culture media on day-3 (19.85%, n=2) or day-5 (20.35%±0.30%, n=3) or day-7 (19.33%±0.84%, n=3) compared to continuous supplementation (20.91%±1.61%, n=3; average±SEM) was observed (FIG. 6A and FIG. 68).

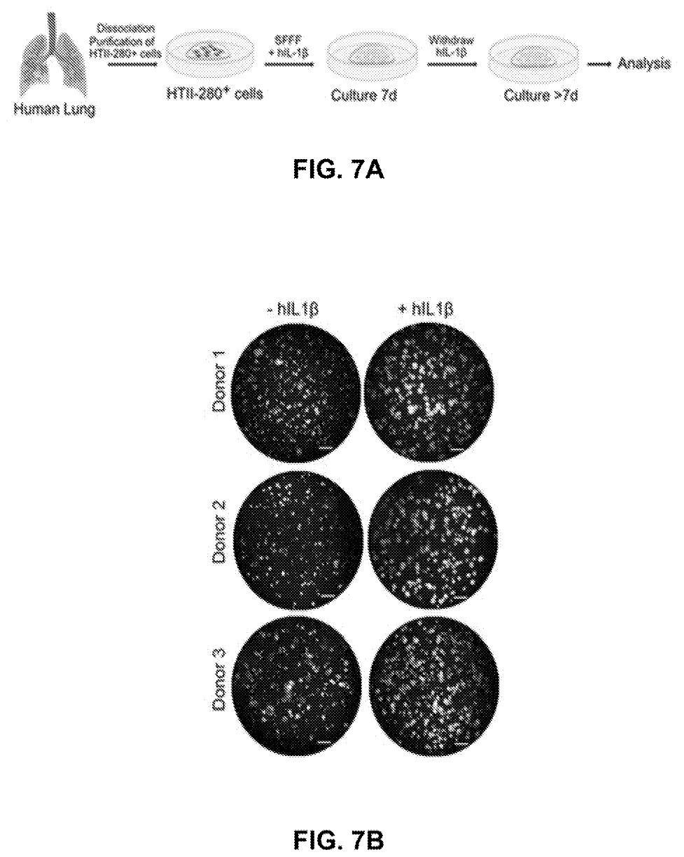

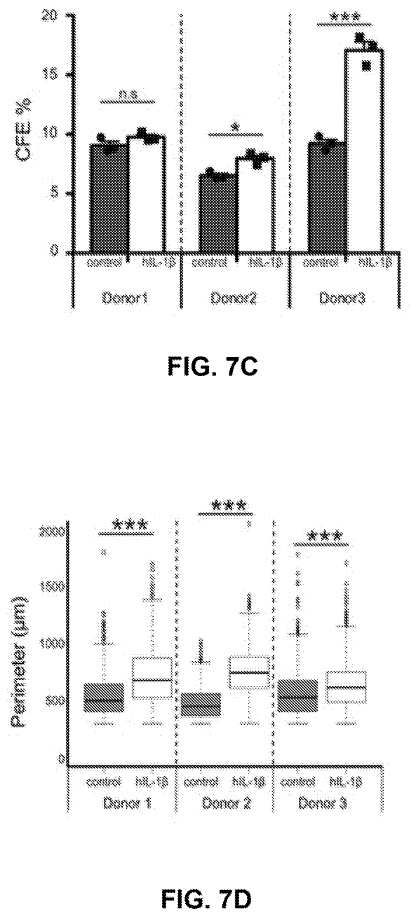

The impact of human IL-1β was also tested in human alveolosphere culture. Human IL-1β was removed from medium containing human alveolospheres from three individual donors at day 7 and cultured for an additional 7-15 days (FIG. 7A). Treatment with IL-1β significantly enhanced organoid numbers and the size (which reflects the growth rate) (FIG. 7B, FIG. 7C, and FIG. 7D).

Taken together, these data revealed that transient IL1β stimulation in the early stages of organoid cultures is sufficient to replace fibroblasts when AEC2s are cultured in the newly established serum-free-feeder-free conditions (here after referred to as Alveo-expansion medium).

### Example 3: AEC2s from Defined Culture Conditions are Functional In Vivo and Ex Vivo

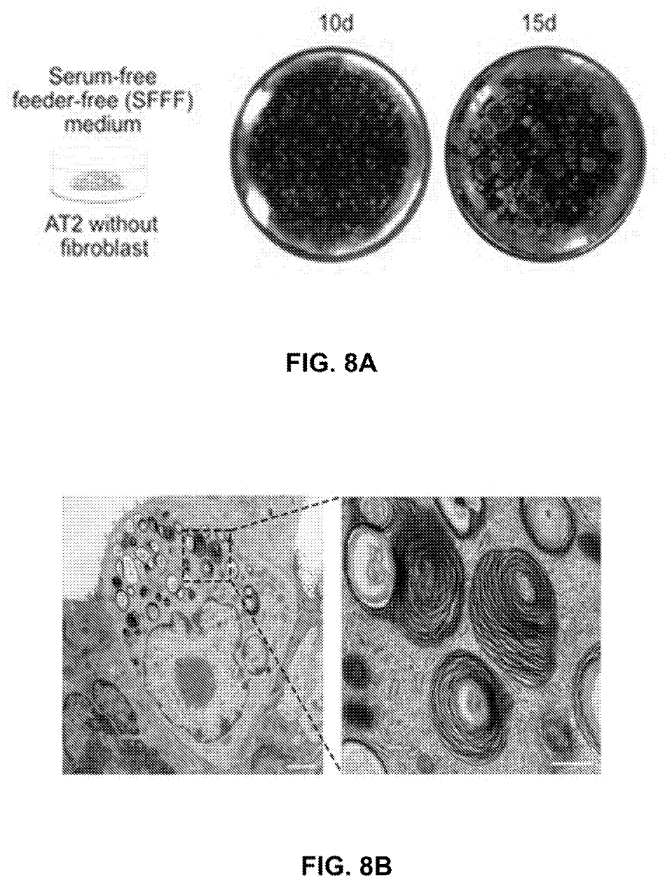

Lamellar body presence is used as a benchmark assay to define AEC2s identity and functions (Beers, et al., 2017, Am. J. Respir. Cell Mol. Biol. 57, 18-27). To test the presence of lamellar bodies in our organoid culture-derived AEC2s, electron microscopy analysis was performed. Schematic and representative images of alveolospheres derived from labeled (tdTomato+) cells cultured in SFFF medium at 10 and 15 days are shown in FIG. 8A. Numerous lamellar bodies in AEC2s from the organoids (FIG. 8B).

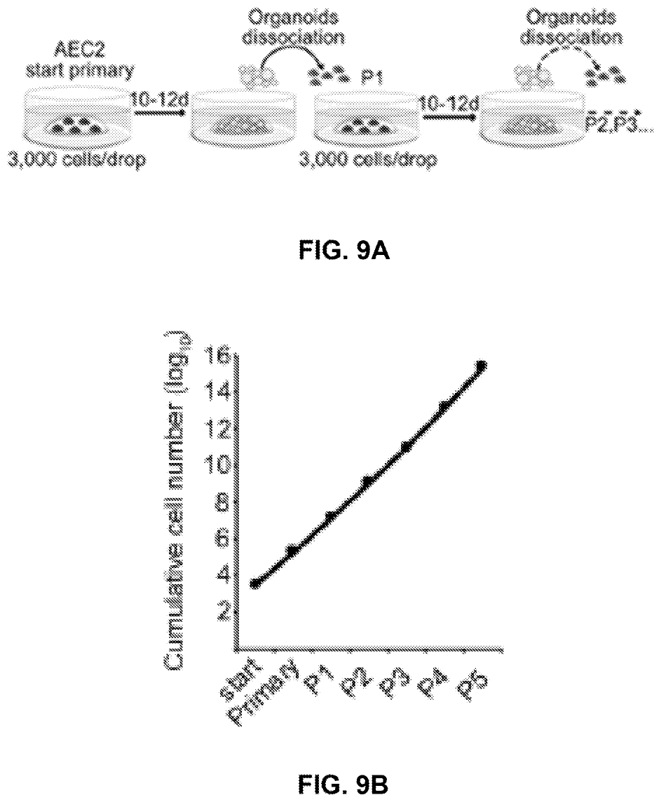

To test whether mouse AEC2s can be passaged, organoid-derived cells were sub-passaged for over 5 passages. Quantification for cell numbers over 5 passages revealed an exponential increase in the total number of cells over the passages revealing that they can self-renew and maintain the expression of markers (FIG. 9A and FIG. 9B).

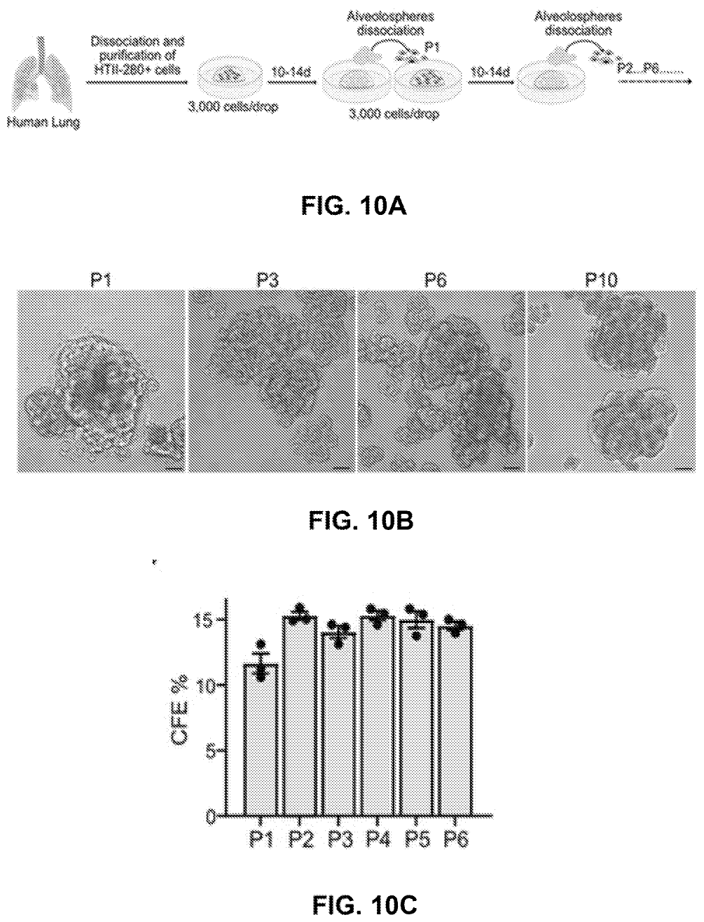

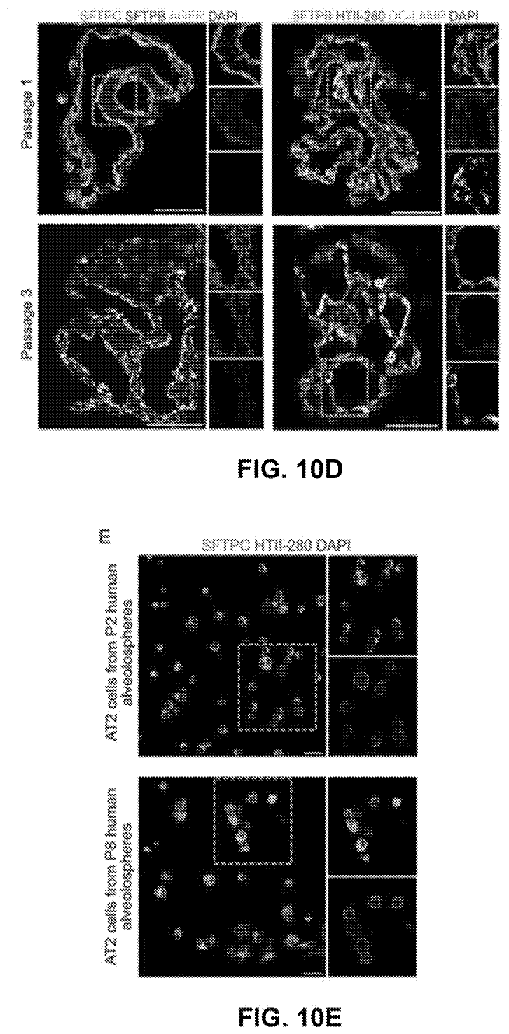

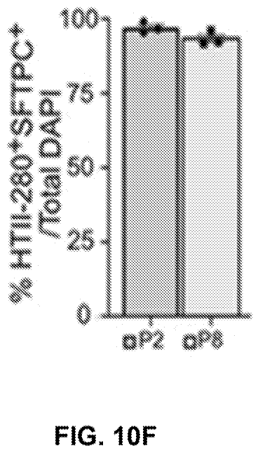

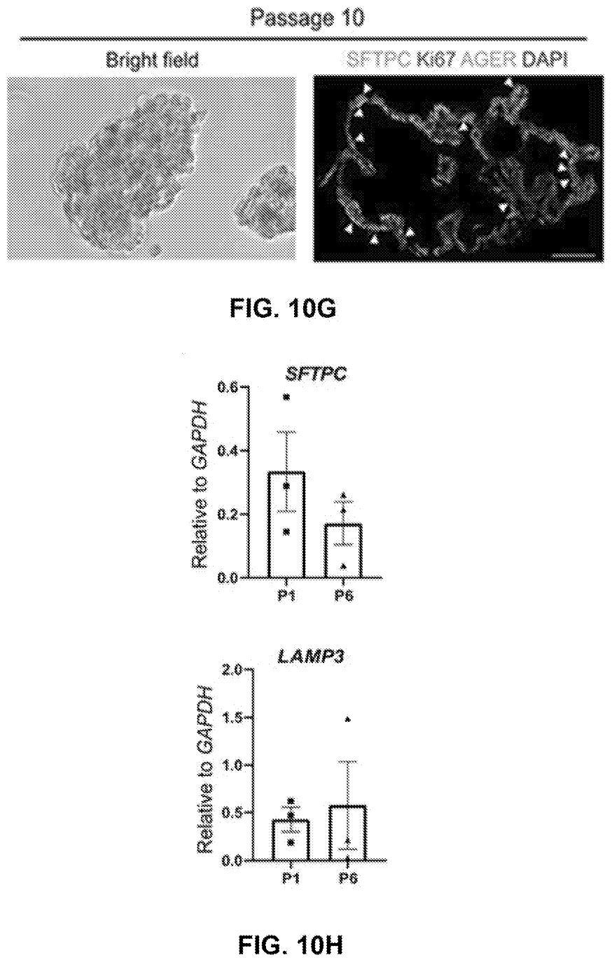

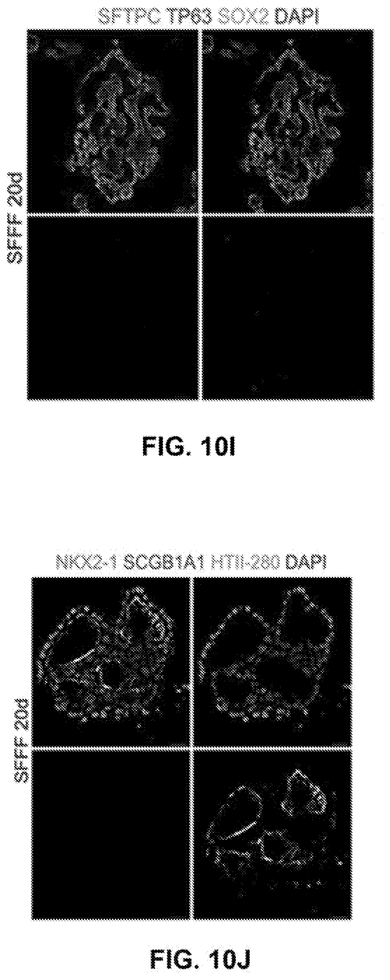

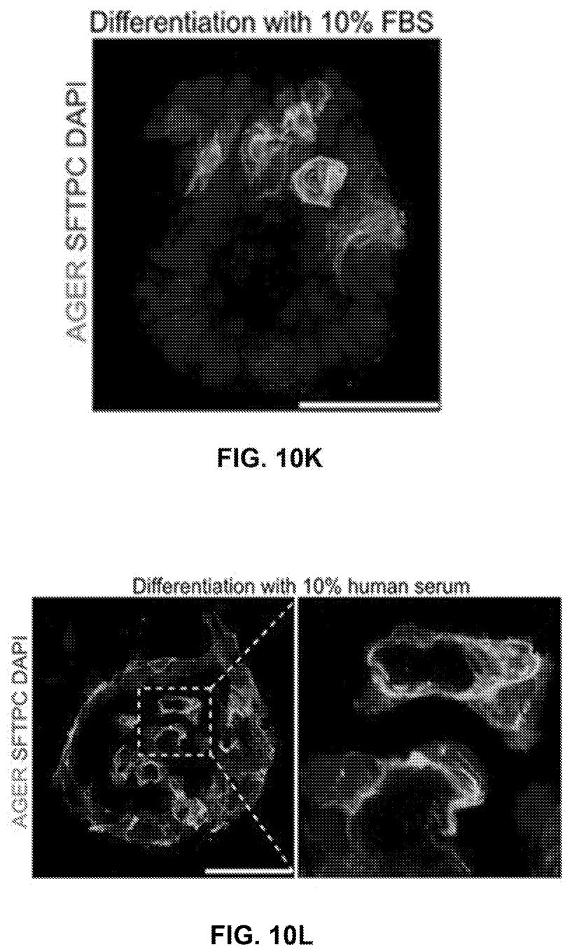

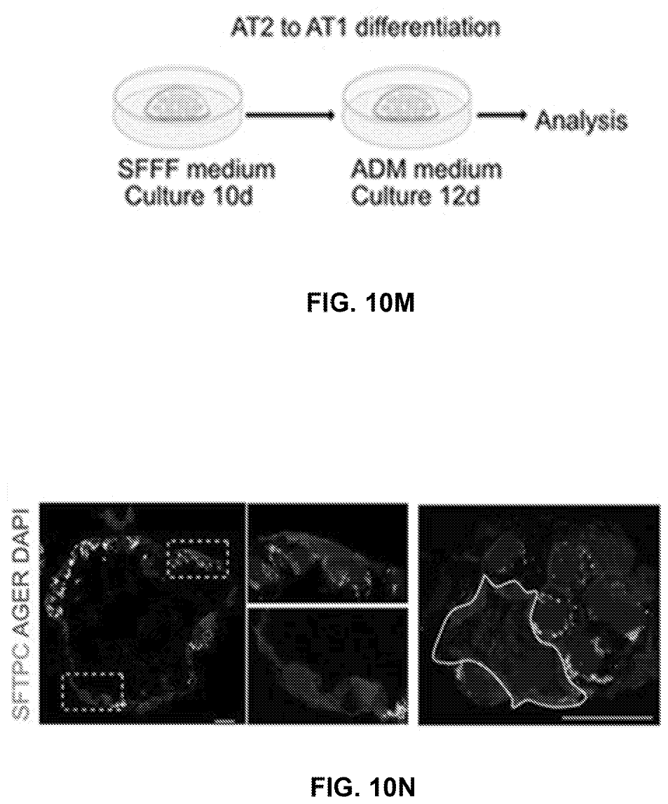

To test whether human AEC2s can be passaged, HTII-280+− cells were isolated and purified from human donors (FIG. 10A). Imaging and quantification of cell numbers in organoids cultured in SFFF medium maintained expression of AEC2s markers and self-renewal for several passages for over 10 passages (FIG. 10B, FIG. 10C, FIG. 10D, FIG. 10E, and FIG. 10F). Organoids cultured in IL-1β maintained expression of AEC2s markers and self-renewal for several passages (FIG. 10G, FIG. 10H, FIG. 10I, and FIG. 10J). Organoid cultures in IL-1β maintained differentiation potential for several passages (FIG. 10K, and FIG. 10L) and organoids cultured in SFFF medium maintained differentiation potential for several passages for over 10 passages (FIG. 10M, and FIG. 10N).

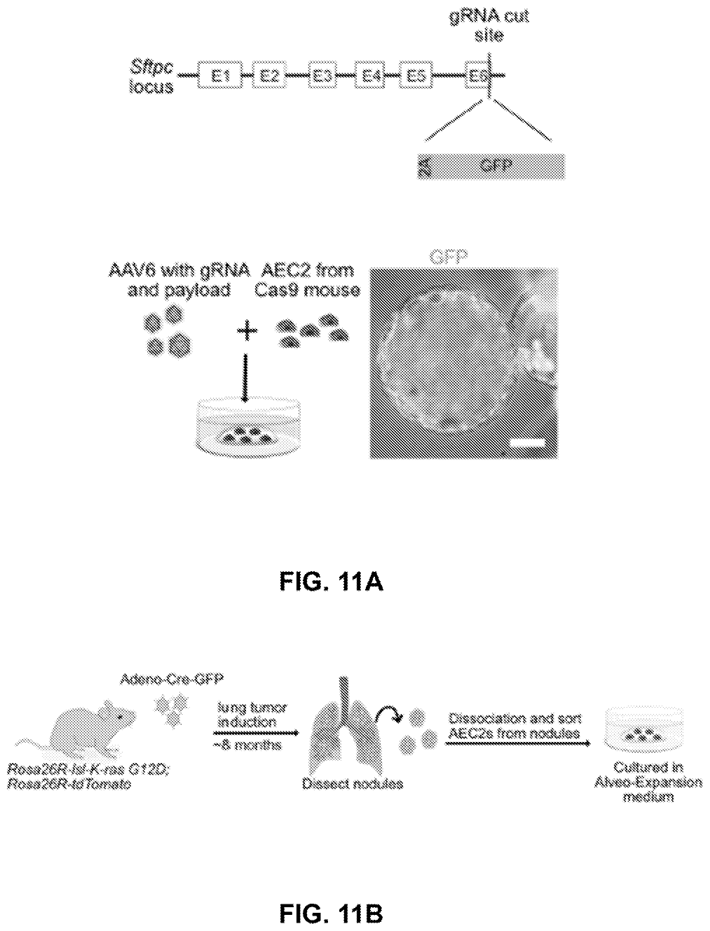

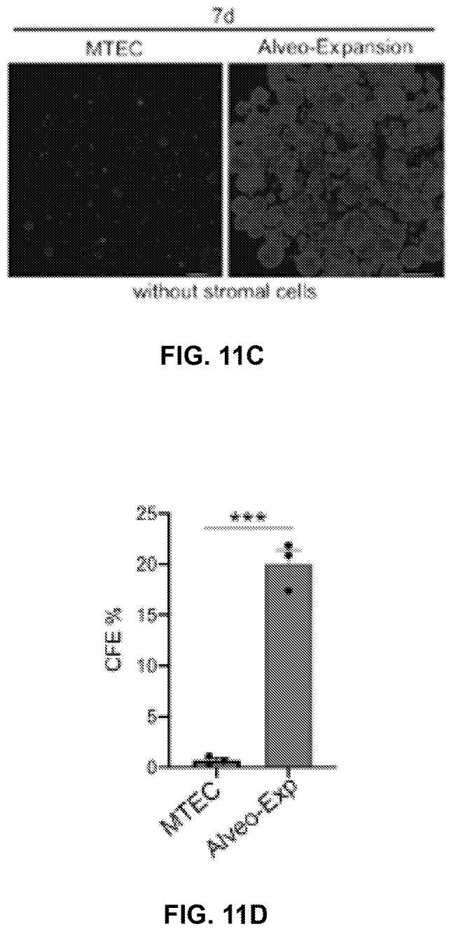

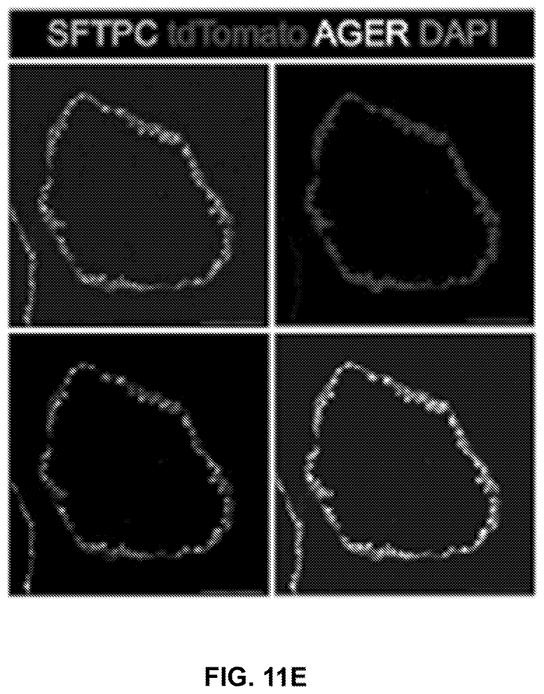

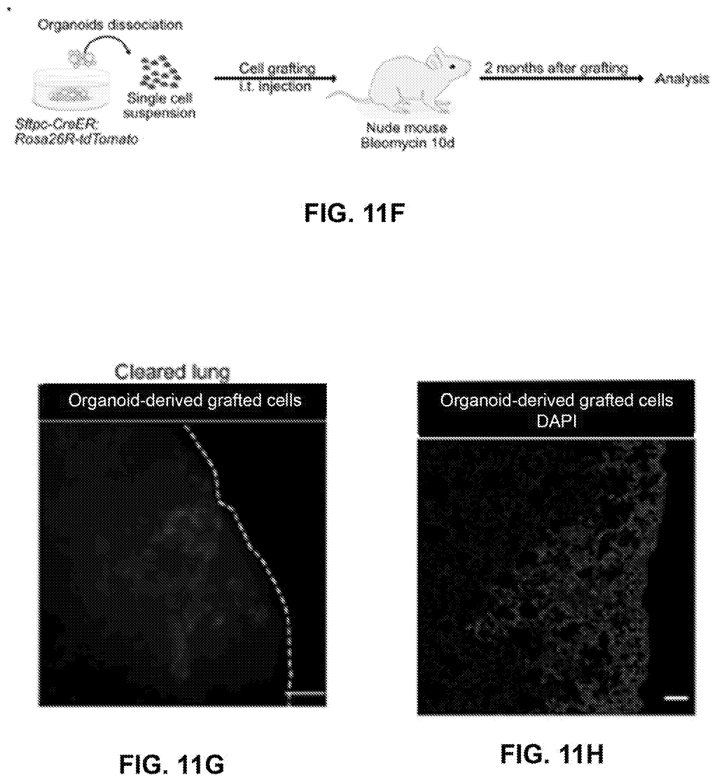

It was then tested whether the organoid cultures are amenable for Cas9/Crispr mediated genome editing. To test this, a recently described homology independent transgene integration (HITI) method to insert a T2A-GFP encoding DNA in the 3′ end of the Sftpc gene coding sequence was used. Successful gene editing was visualized by GFP expression in clonally derived AEC2 organoids (FIG. 11A). These data serve as a proof-of concept that our organoid conditions are amenable for gene editing and disease modeling. Recent studies have used organoid based tumor models to study tumorigenesis ex vivo. Indeed, recent studies have used MTEC medium to culture lung adenocarcinoma cells in the presence of fibroblasts.

To test whether the newly established culture medium is suitable for culturing lung tumor-derived cells in the absence of fibroblasts, tumor nodules were isolated from Kras G12D/tdTomato mice and purified tdTomato+ tumor cells (FIG. 11B). Organoid cultures were setup using these tumor cells in the absence of stromal cells in our newly established medium and directly compared them with MTEC medium. Interestingly, tumor cells developed numerous organoids in the new medium but not in MTEC medium (CFE, 0.7%±0.2% in MTEC versus 20.0%±1.4% in Alveo-Expansion medium [n=3] on day 5; mean±SEM) (FIG. 11C, FIG. 11D, and FIG. 11E). These data revealed that the newly established medium conditions support tumor cell growth ex vivo even in the absence of stromal cells.

Finally, organoid-derived cells were tested for their ability to engraft in vivo. To test this, tdTomato labeled cell suspension was intratracheally injected into lungs of nude mice that were administered with bleomycin to damage lungs (FIG. 11F). Two months after injection, patches of tdTomato+ cell patches in the injured lungs were observed (FIG. 11G and FIG. 11H). Immunofluorescence and histological analysis further revealed that engrafted cells integrated into the regenerated tissues and expressed markers of AEC2 and AEC1s, indicating successful engraftment of organoid-derived cells (FIG. 11I). Taken together, organoid-derived cells from the newly established resemble in vivo correlates of AEC2s, amenable for gene editing, and can functionally integrate into regenerating tissues in engraftment assays.

### Example 4: Chemically Defined Conditions for AEC2 Maintenance and Differentiation

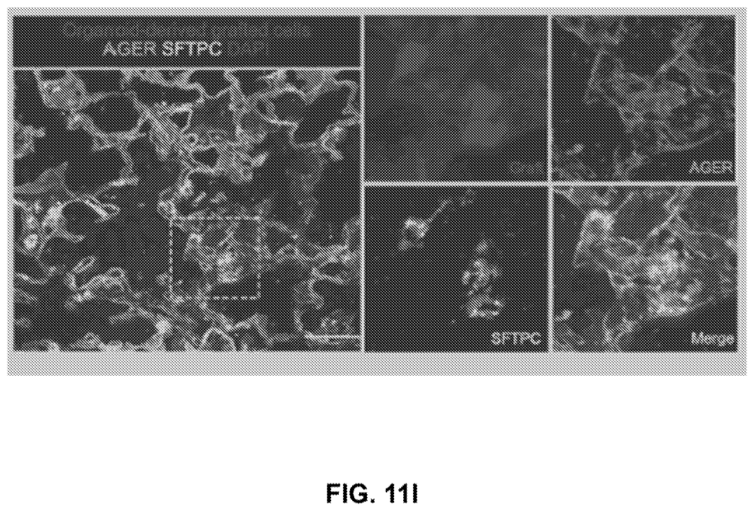

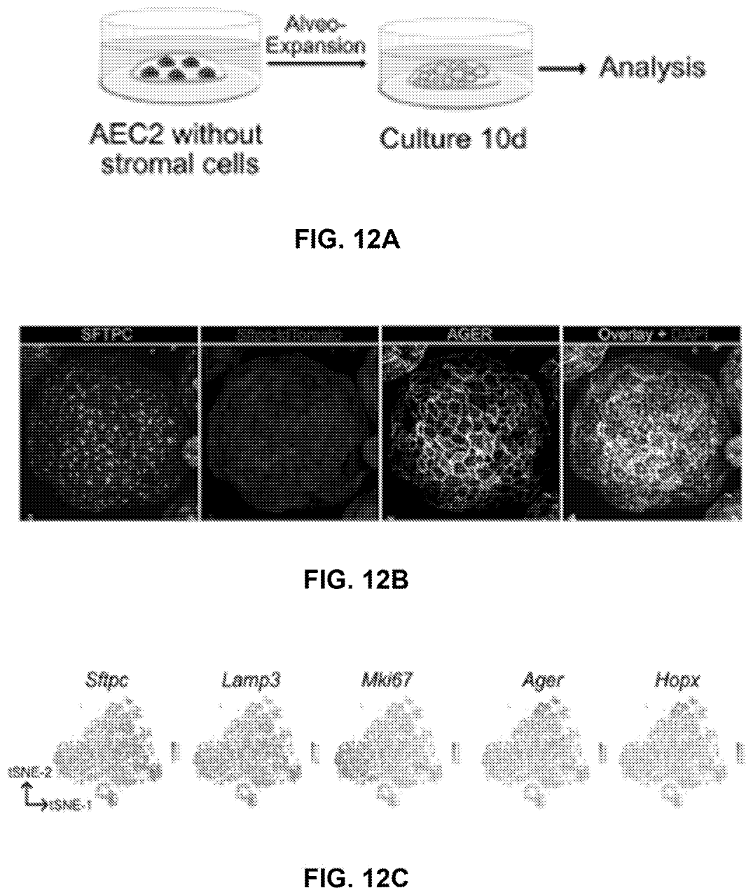

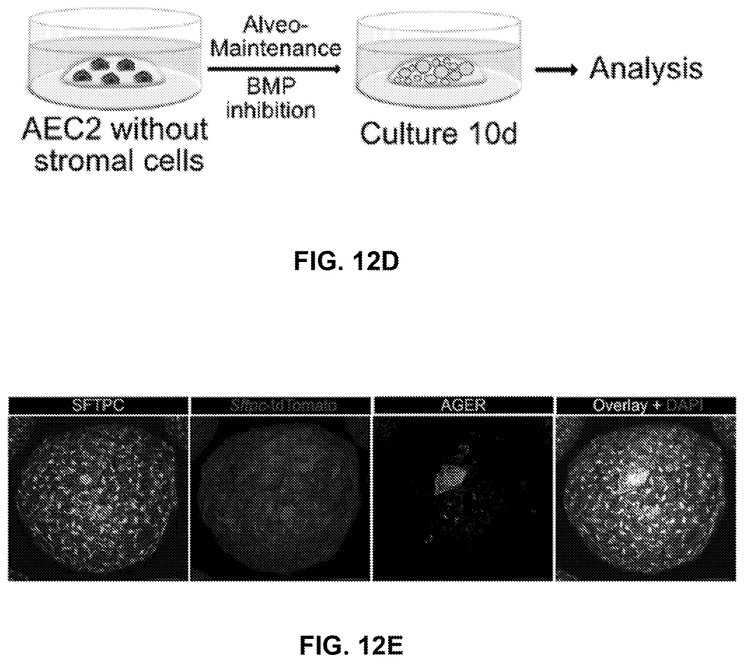

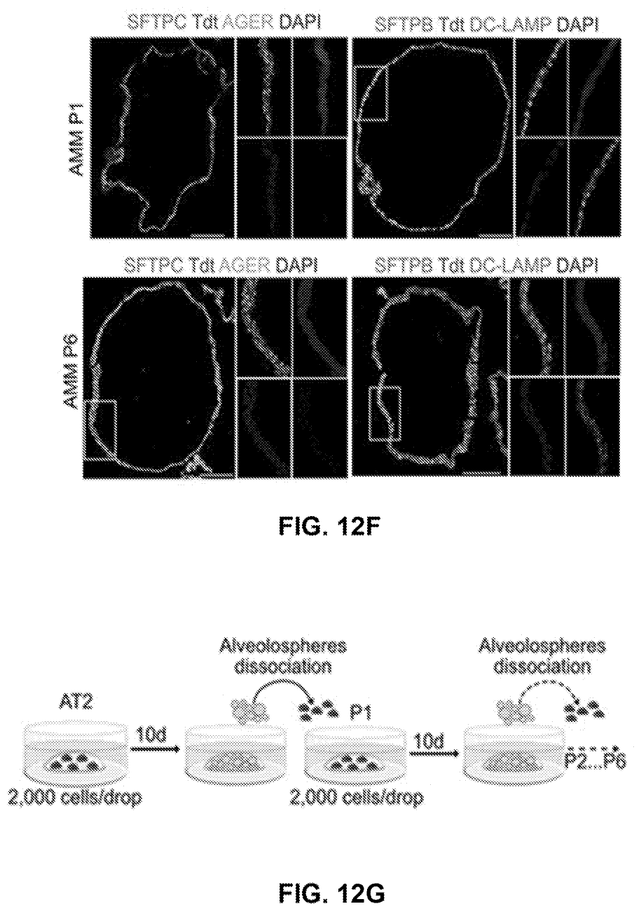

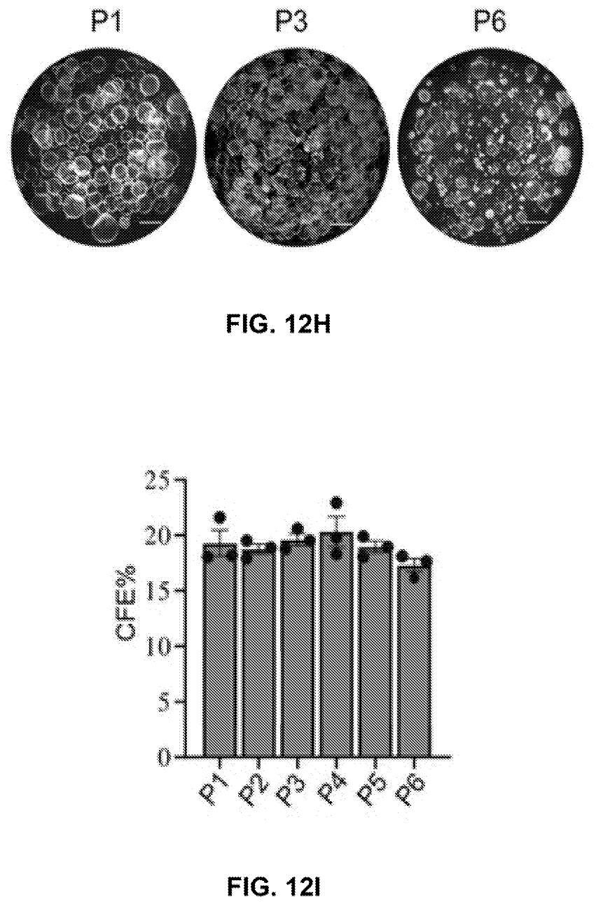

Immunofluorescence analysis for AEC2 and AEC1 markers on organoids derived from Alveo-expansion medium indicated that most of the cells (˜80%) co-expressed AEC2 as well as AEC1 markers, indicating that these conditions are promoting both AEC2 and AEC1 identities in the same cells (FIG. 12A, FIG. 12B, and FIG. 12C). Interestingly, the scRNA-seq guided epithelial-stromal cell interactome revealed that ligands (Bmp4), and inhibitors (Fst, Fst1, and Grem1) of BMP signaling are expressed in AEC2 and stromal cells, respectively (FIG. 2D and FIG. 2E). Furthermore, recent studies have implicated BMP signaling in AEC2 to AEC1 differentiation (Chung et. al., 2018, Development 145, dev163014: Lee et al., 2014, Cell 156, 440-455). It was therefore hypothesized that in the absence of stromal cells, BMP ligands produced by AEC2 cells act in an autocrine manner and induce differentiation.

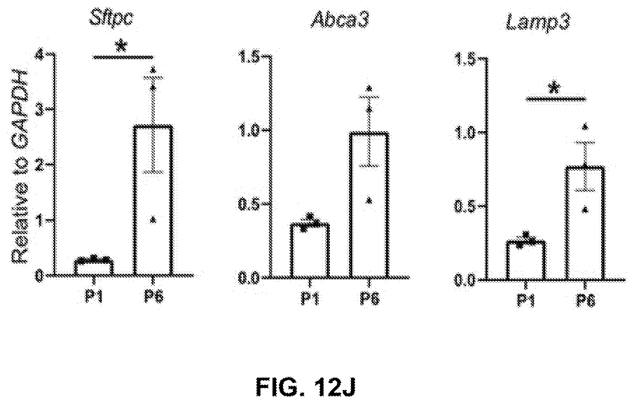

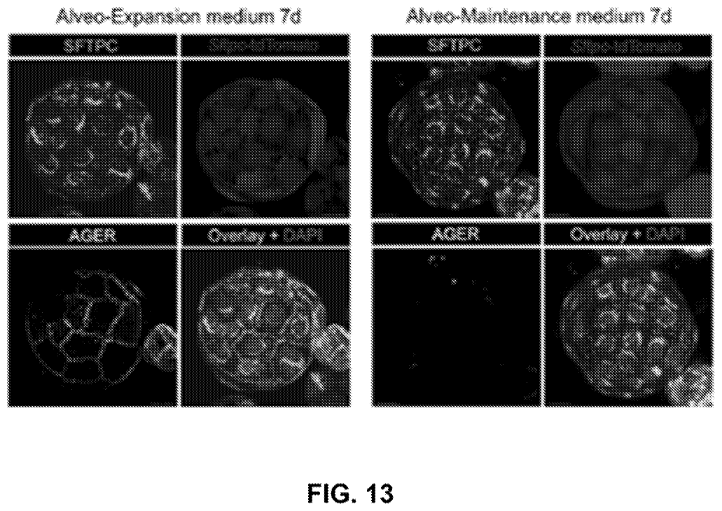

To test whether inhibition of BMP signaling blocks emergence of AEC1 identity while maintaining AEC2 cell identity, the Alveo-expansion medium was supplemented with inhibitors of BMP signaling (Noggin and DMH1). Whole mount immunostaining and quantification for SFTPC and RAGE revealed that a dramatic reduction in the number of RAGE-expressing organoids (down to 30%) and the number of RAGE-expressing cells (>5%) in each organoid (FIG. 12D and FIG. 12E). Marker analysis for AEC2s and AEC1 further revealed that organoids cultured in alveolar maintenance medium maintained self-renewal properties over 6 passages (FIGS. 12F-12J). These data revealed that Alveo-expansion media with BMP inhibitor (referred to as Alveo-Maintenance medium) maintains AEC2 cell identity while repressing the induction of AEC1 cells in these organoids (FIG. 13).

These data are in line with previous studies that BMP signaling is necessary for AEC1 differentiation. However, complete differentiation of AEC2 to AEC1 cells when organoids were treated with BMP4 ligand was not observed, suggesting that BMP signaling is necessary but not sufficient to induce differentiation.

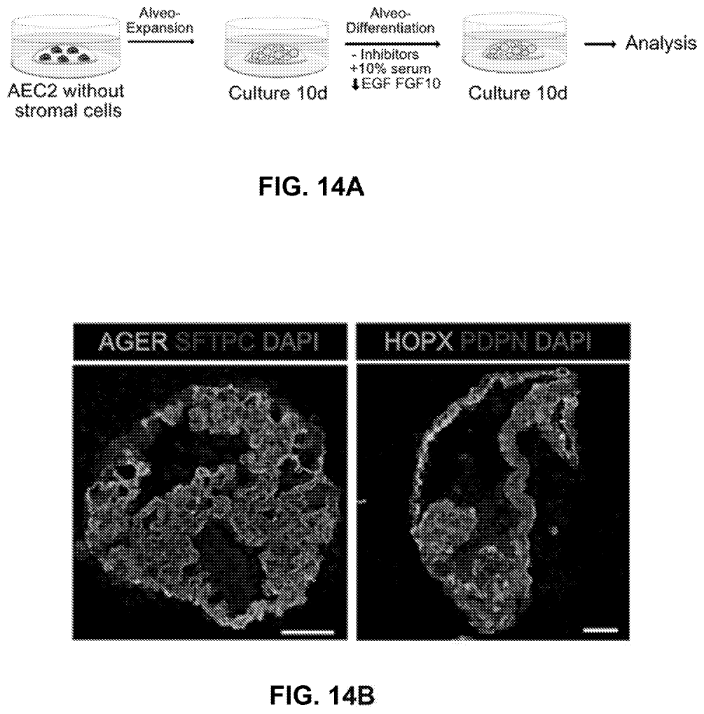

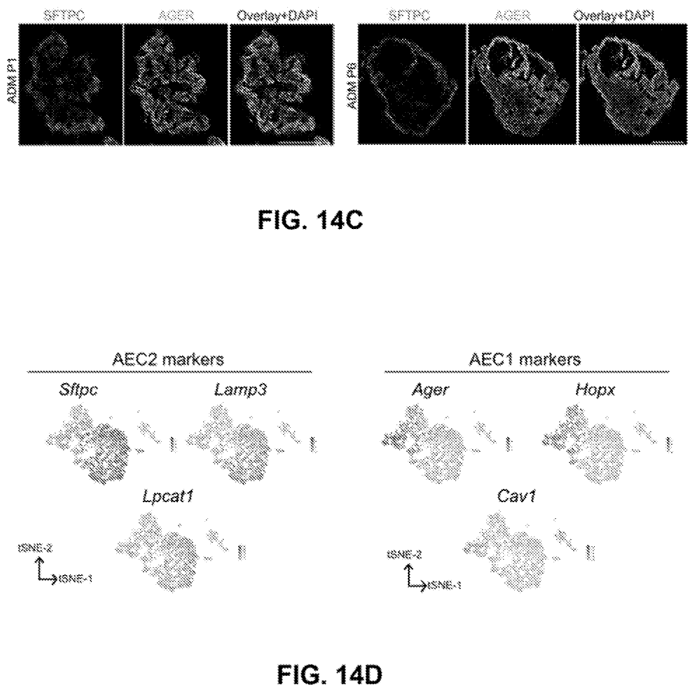

To find factors that can induce differentiation of AEC2 into AEC1, different molecules were tested (Dexamethasone, T3, BMP4, TGFs, and IBMX (phosphodiesterase inhibitor)) that were previously thought to promote differentiation. In the above experiments using scrum containing MTEC medium, spontaneous differentiation of AEC2 cells was observed. Therefore, it was thought that decreasing or completely eliminating the factors that promote AEC2 growth in combination with low amounts of serum might stimulate differentiation. To test this, AEC2 from mouse lungs were cultured in maintenance medium for 10 days, then inhibitors of TGFs and p38 kinase were removed, the amount of EGF and FGF (by 10-fold) was decreased, and 10% fetal bovine serum was added to the medium (here after referred to as Alveo-Diff medium) and cultured cells for 10 days (FIG. 14A). A significant increase in the number of RAGE, HOPX, and T1a+ cells in Alveo-Diff medium was observed. Single cell transcriptome analysis on Alveo-Diff media derived cells clearly indicated that that these organoids are composed of numerous AEC1 cells. Of note, a significant decrease in the number of proliferating AEC2 cells was observed, indicating that factors present in serum may prevent AEC2 proliferation, further asserting the importance of Alveo-expansion medium that was developed and described above (FIG. 14B, FIG. 14C, and FIG. 14D).

Taken together, and as described herein, culture conditions for the expansion, maintenance and differentiation of AEC2s in organotypic cultures have been formulated.

### Example 5: Chemically Defined (Serum Free) Conditions for Alveolar Stem Cell Differentiation

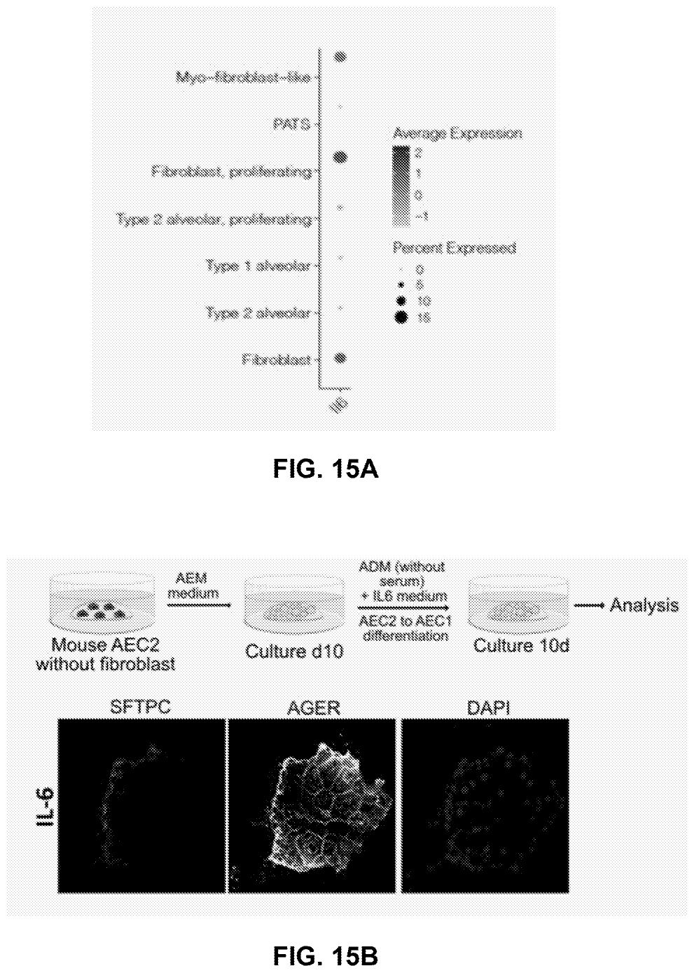

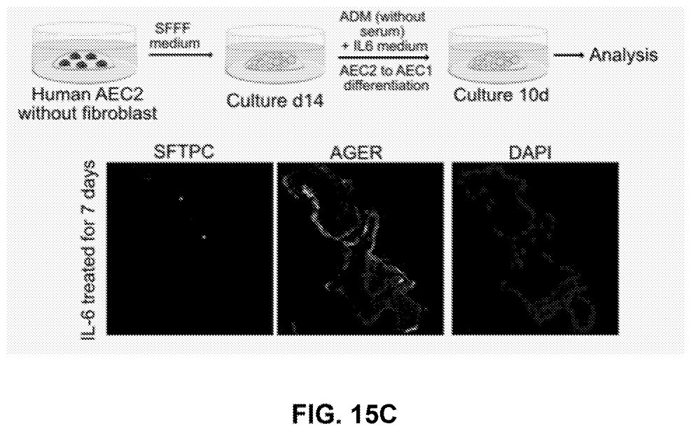

To identify factors that can induce AEC2s differentiation into AEC1, scRNA-seq data were mined from organoids co-cultured with fibroblasts. Molecules that are expressed in fibroblasts that can potential binds on receptors in AEC2s were searched. An enrichment for ILS transcripts was identified in fibroblasts (FIG. 15A). Previous studies have revealed that AEC2s express IL6 receptors (Zepp et al., 2017, Cell, 170(6):1134-1148). To test whether IL6 is sufficient to induce AEC2s differentiation, mouse AEC2s were cultured in alveolar maintenance medium for 10 days to expand AEC2s in organoid cultures. Then, organoids were treated with Alveolar differentiation medium that lacks scrum but supplemented with IL6 (20 ng/mL) and cultured them for additional 10 days. Immunostaining analysis for organoids cultured in this medium revealed a strong expression of AEC1 markers including, AGER (FIG. 15B). Similarly, human AEC2s were cultured in SFFF medium for 14 days prior to replacing medium with ADM (without serum) supplemented with IL6 (20 ng/nL) (FIG. 15C). These studies further revealed that IL6 treatment is sufficient to induce differentiation of both mouse and human AEC2s in to AEC1 in cultures.

### Example 6: Alveolosphere-Derived AT2s are Permissive to SARS-CoV-2 Infection

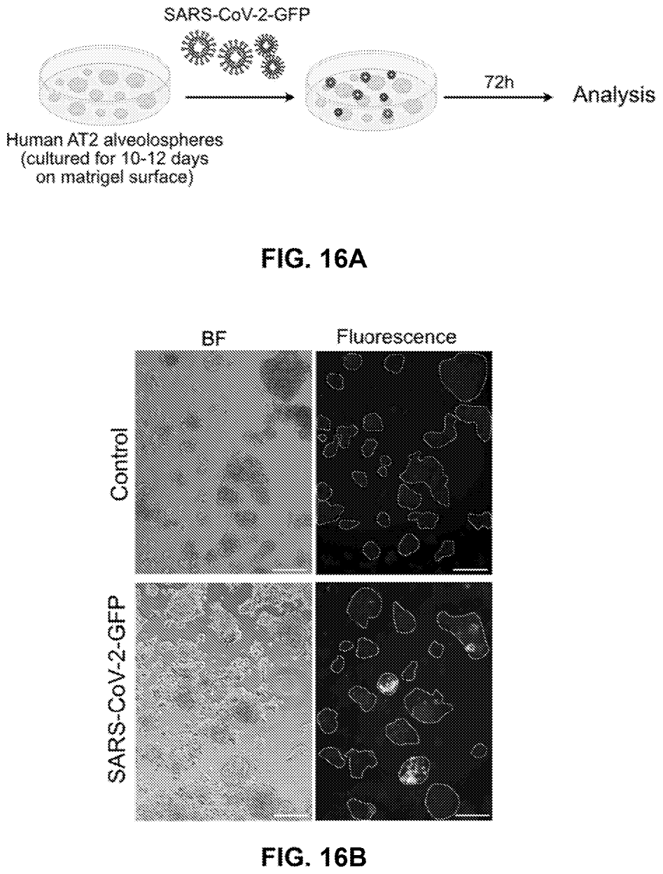

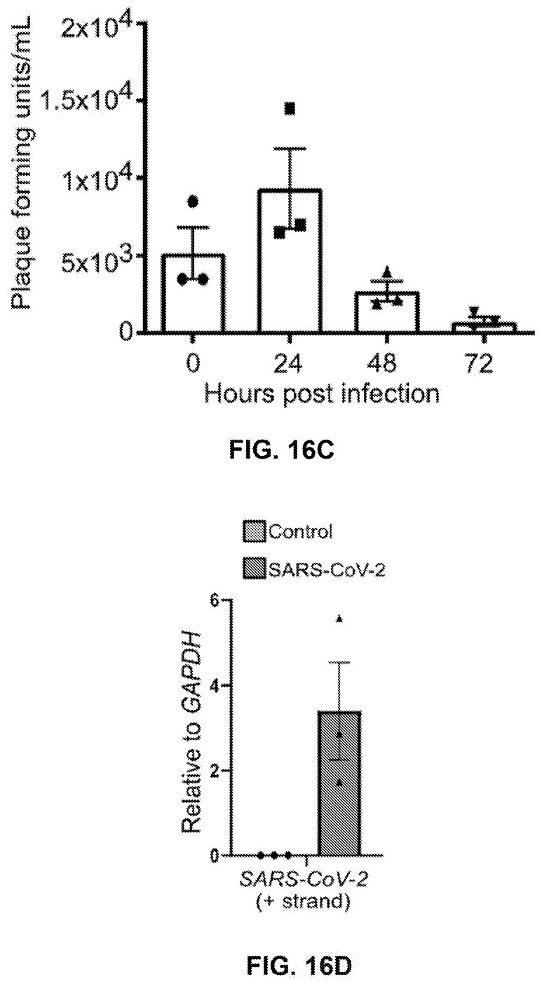

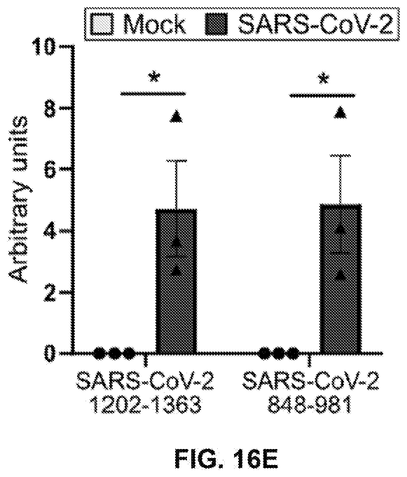

To test whether SARS-CoV-2 can infect alveolosphere-derived AT2 cells, a recently developed reverse-engineered SARS-CoV-2 virus harboring a GFPfusion protein was utilized (Hou et al., 2020, Cell, 182(2):429-446). Human alveolospheres were cultured on matrigel surface in SFFF media (lacking IL1β) for 10-12 days, incubated with SARS-CoV-2-GFP for 2 h, washed with PBS to remove residual viral particles and then collected for analysis over 72 h (FIG. 16A). GFP was detected as early as 48 h post infection in virus exposed but not in control alveolospheres (FIG. 16B). Subsequent plaque forming assays using culture supernatants revealed that viral release peaks at 24 h but later declined (FIG. 16C). This observation was consistent across cells from three different donors. Of note, a significant number of viral particles immediately after infection despite numerous washes with PBS were observed. This result was likely due to the entrapment of virus in the Matrigel. Nevertheless, the viral titer increased at 24 hpi demonstrating that SARS-CoV-2 productively replicates in AEC cells (FIG. 16C). Quantitative RT-PCR further revealed the presence of viral RNA in SARS-CoV-2 infected cells compared to controls (FIG. 16A). To further confirm virus replication, qRT-PCR was performed using primer that specifically recognize minus strand of the virus. Indeed, viral replication in alveolosphere cultures was observed (FIG. 16E).

### Example 7: AT2s Activate Interferon and Inflammatory Pathways in Response to SARS-CoV-2 Infection

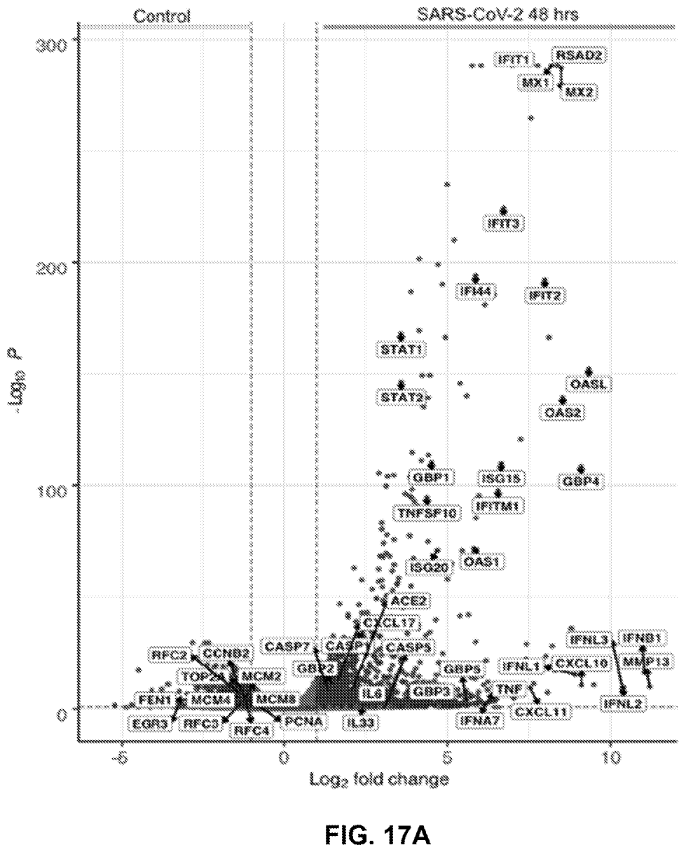

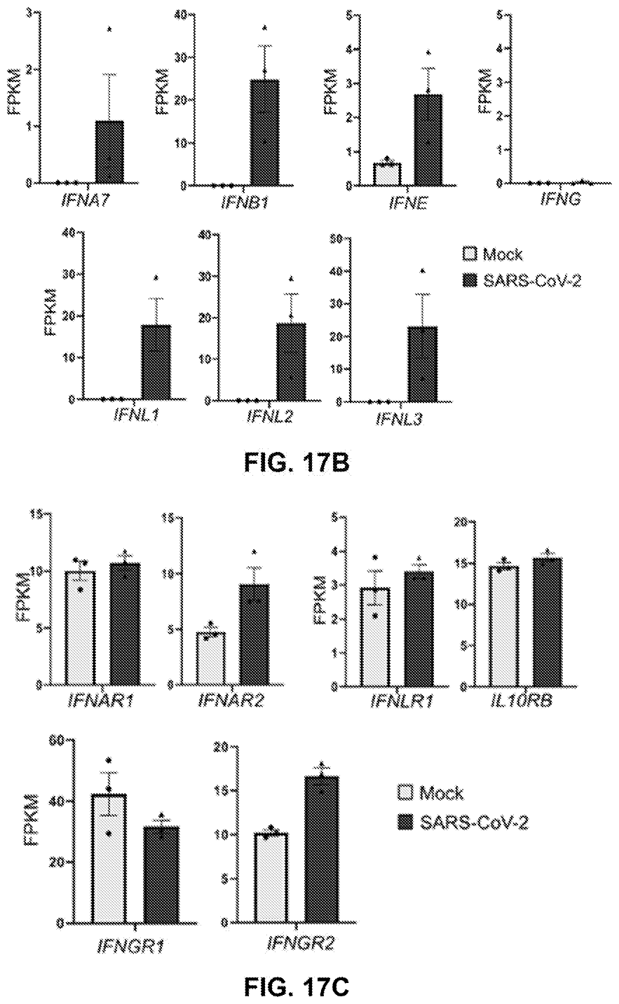

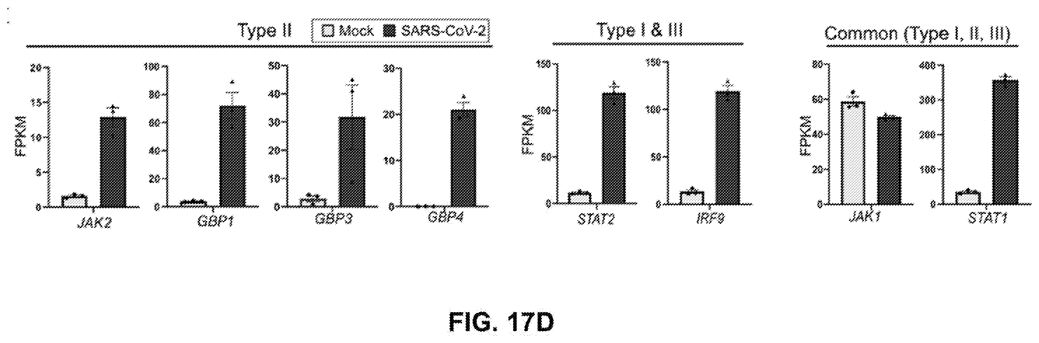

To gain insights into the response of AT2s to SARS-CoV-2 (wild type), unbiased genome-wide transcriptome profiling on alveolospheres cultures 48 h after infection was performed. Of all the sequenced reads, viral transcripts accounted for 4.7% and human transcripts accounted for 95.3%, indicating that virus was propagating in AT2s. Previous studies have shown that in response to viral infection, target cells typically produce Type I (IFN-I) and Type III (IFN-III) interferons (a/b and λ, respectively) which subsequently activate targets of transcription factors IRF, STAT1/2 and NF-κB including interferon stimulated genes (ISGs), inflammatory chemokines, and cytokines that go on to exert antiviral defense mechanisms (Barrat et al., 2019, Nat. Immunol. 20, 1574-1583). It was therefore significant that differential gene expression analysis of infected versus uninfected alveolospheres revealed enrichment of transcripts related to general viral response genes, including multiple interferons (IFNs) and their targets. Specifically, SARS-CoV-2 infected AT2s were enriched for transcripts of Type I IFNs (IFNA7, IFNB1 and IFNE) as well as Type III IFNs (IFNL1, IFNL2 and IFNL3) but not Type II IFNs (IFNG) ligands (FIG. 17A and FIG. 17B). Receptors for Type I (IFNAR1 and IFNAR2), Type II (IFNGR1 and IFNGR2) and Type III (IFNLR1 and IL10RB) IFN were expressed in control AT2 cells and a modest increase was found for IFNAR2 and IFNGR2 after SARS-CoV-2 infection (FIG. 17A and FIG. 17C) (Platanias, 2005; Syedbasha and Egli, 2017).

These data indicate that in response to SARS-CoV-2 infection, AT2s produce Type I and III IFN ligands, which can potentially act via either by autocrine or paracrine (neighboring AT2s) mechanisms to activate their cognate receptors. Indeed, a large number of IFN target genes including IFN-stimulated genes (ISOs), IFN-induced protein-coding genes (IFIs) and IFN-induced protein with tetratricopeptide repeats-coding genes (IFITs), were up-regulated in SARS-CoV-2 infected AT2s (FIG. 17A and FIG. 17D). Additionally, key transcription factors STAT1 and STAT2 that are known to be components of the signaling pathways downstream of IFN receptors were also upregulated in infected AT2 cells.

Pathway analysis revealed all three classes of IFN targets were upregulated, but the most prominent were type I and type II IFN signaling. Despite the absence of type II IFN ligands (IFNG) a significant upregulation of canonical targets of IFNγ-response mediators in SARS-CoV-2 infected AT2 cells was observed (FIG. 17A and FIG. 17D). This finding suggests that there is a significant overlap of downstream targets and cross-talk between different classes of IFN pathways, as described previously (Barrat et al., 2019; Bartee et al., 2008). Other prominent upregulated genes include chemokines (CXCL10, CXCL11 and CXCL17) and programmed cell death-related genes (TNFSF10, CASP1, CASP4, CASP5 and (ASP7) (FIG. 17A). In contrast, a significant downregulation of transcripts associated with DNA replication and cell cycle (PCNA, TOP2A, MCM2, and CCNB2) in infected AT2 cells was observed (FIG. 17A). Selected targets (IFNA7, IFNB1, IFNL1, IFIT1, IFIT2, IFIT3, IL1A, IL1B, IL6, CSCL10) were validated using independent quantitative RT-PCR assays at early (48 h) and late (120 h) time points post infection. Taken together, transcriptome analysis revealed a significant upregulation of interferon, inflammatory and cell death signaling, juxtaposed to downregulation of proliferation-related transcripts, in alveolosphere-derived AT2s in response to SARS-CoV-2.

### Example 8: SARS-CoV-2 Infection Induces Loss of Surfactants and Pneumocyte Death

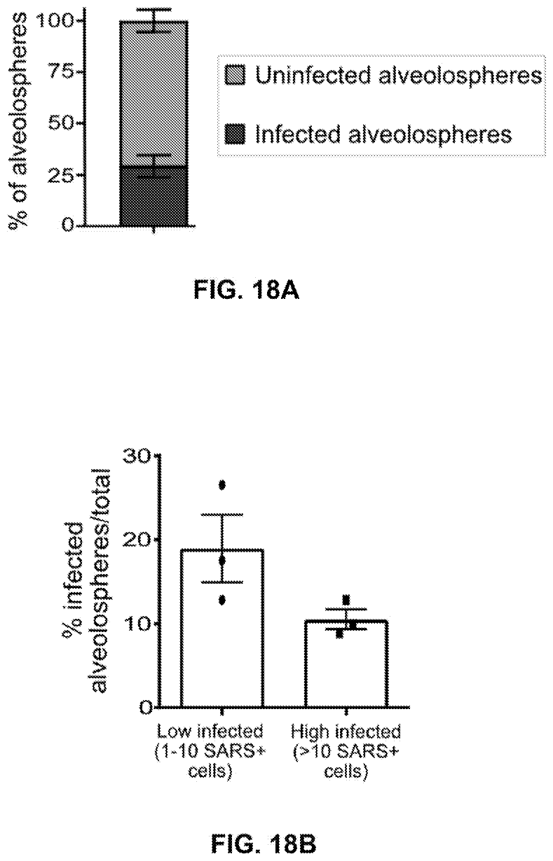

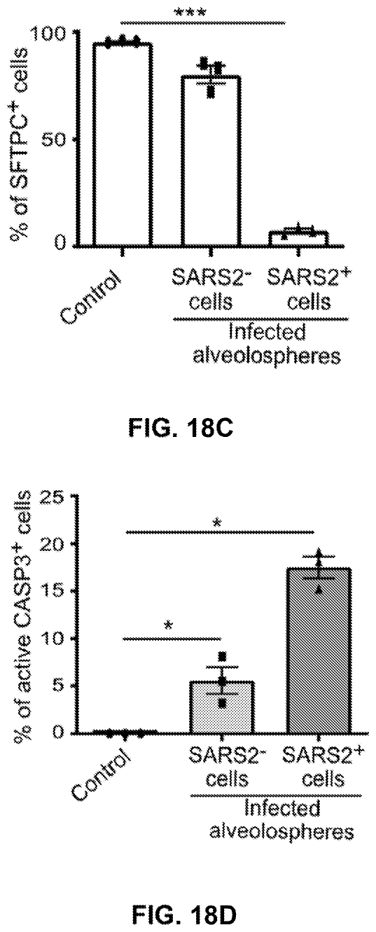

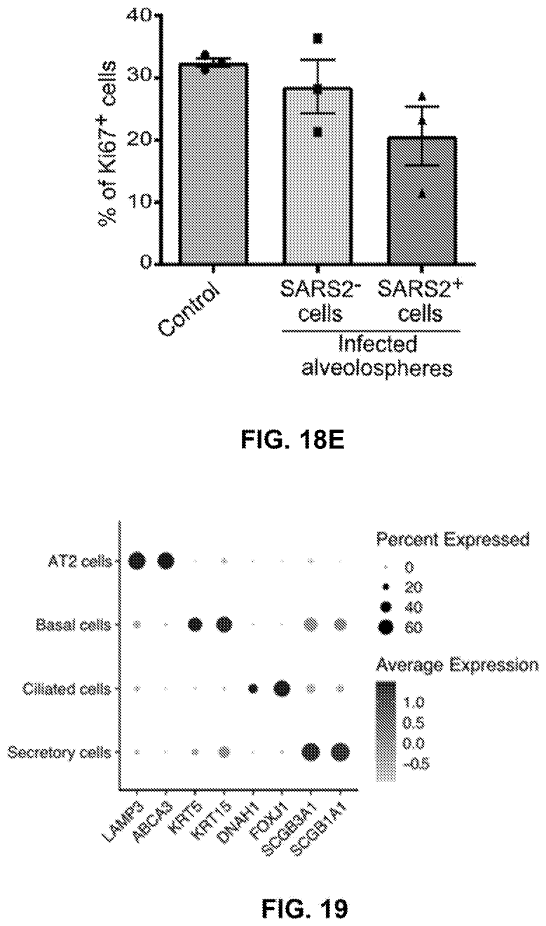

To gain further insights into how primary AT2 cells respond early to SARS-CoV-2 infection, cellular changes in alveolospheres 24 hours to 72 hours after infection were analyzed using immunohistochemistry. Quantification of infected alveolospheres revealed that 29.22% are SARS: (FIG. 18A). Immunostaining revealed co-expression of GFP and SARS-CoV-2 spike protein in infected alveolospheres. Variation in the number of GFP: cells in each alveolosphere was found. Therefore, alveolospheres were broadly categorized into low (1-10 cells) and high (>10), depending on the number of SARS+ cells in each alveolosphere (FIG. 18B). Next, analyses for AT2 cell markers, including SFTPC, SFTPB and HTII-280, revealed a dramatic loss or decrease in the expression of surfactant proteins SFTPC and SFTPB in infected cells (GFP+ or SARS+) but not in control alveolospheres (FIG. 18C). Of note, HTII-280 expression was unchanged as visualized by immunostaining on SARS-CoV-2 infected human alveolospheres. The loss of surfactant protein expression was more apparent in high infected alveolospheres as visualized by immunostaining. Some of the GFP, cells showed a slightly elongated morphology, resembling that of AT1 cells but immunostaining for AT1 cell markers revealed that infected cells did not differentiate into AT1 cells as visualized with co-immunostaining to detect SARS-CoV-2 and AGER. These data are in accord with our scRNA-seq data that AT2s downregulate surfactants expression in response to SARS-CoV-2 infection.

Histopathological evidence suggests that there is a loss of alveolar parenchyma in COVID-19 lungs (Huang et al., 2020, Lancet Lond. Engl. 395, 497-506). To test whether SARS-CoV-2 infection induces cell death, immunostaining for active caspase 3, a marker for apoptotic cells was performed. Apoptotic cells were found in alveolospheres exposed to virus but not in controls, suggesting that AT2 cells undergo cell death in response to SARSCoV-2 infection. Significantly, cell death was observed in both SARS+ and SARS− cells suggesting a paracrine mechanism inducing cell death in uninfected neighboring cells (FIG. 18D). Furthermore, immunostaining for Ki67, a marker for proliferating cells revealed no apparent difference in overall cell replication in virus exposed alveolospheres compared to controls (FIG. 18E). Taken together, these data show that SARS-CoV-2 infection induces downregulation of surfactant proteins and an increase in cell death in AT2 cells via both cell autonomous and non-autonomous mechanisms.

### Example 9: Transcriptome-Wide Similarities in AT2s from SARS-CoV-2 Infected Alveolospheres and COVID-19 Lungs

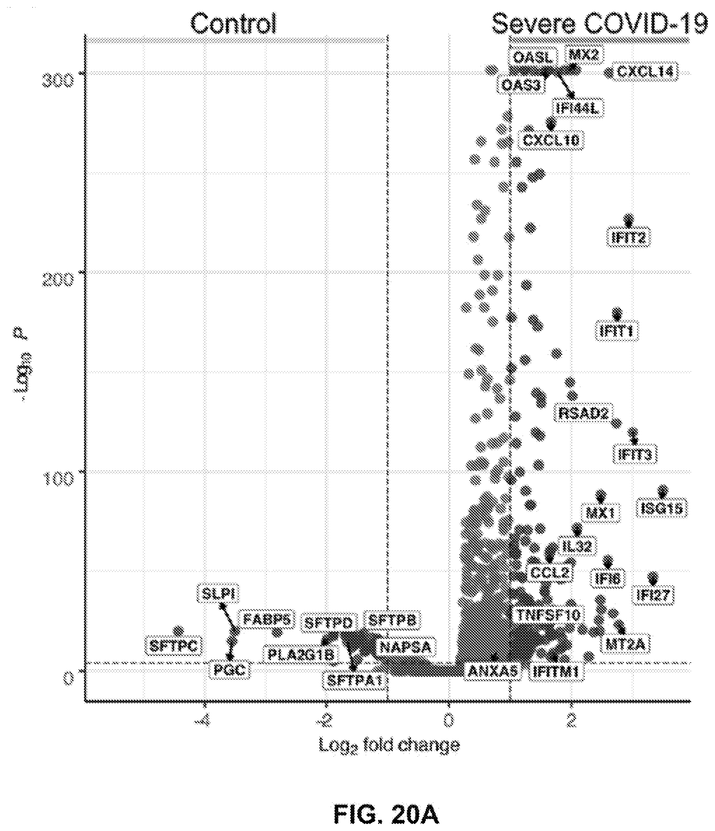

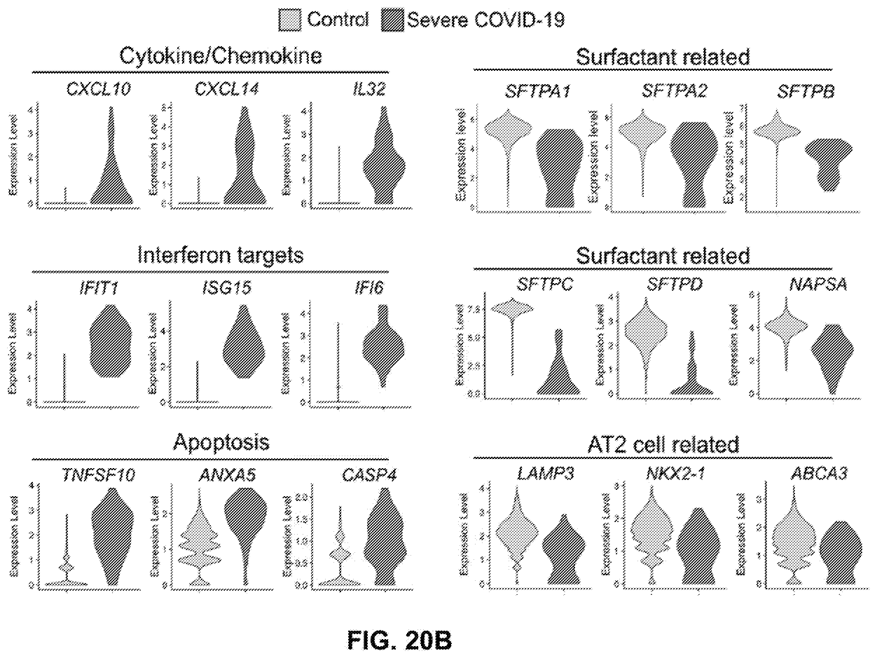

To directly compare SARS-CoV-2 induced responses in AT2s in alveolospheres to changes seen in COVID-19 lungs, a publicly available scRNA-seq dataset from bronchoalveolar lavage fluid (BALF) obtained from six severe COVID-19 patients was utilized (Bost et al., 2020, Cell, 181(7):1475-1488; Liao et al. 2020, Nature Medicine, 26:842-844). First, the gene expression profiles of AT2s from COVID-19 patient lungs with AT2 cells from healthy lungs were compared (FIG. 19). Significant upregulation of chemokines (CXCL10, CXCL14, and IL32), interferon targets (IFIT1, ISG15, and IF16), and cell death (TNFSF10, ANXA5, and CASP4) pathway related transcripts in COVID-19 patient AT2 cells were found (FIG. 20A and FIG. 20B). Intriguingly, surfactant genes including SFTPA1, SFTPA2, SFTPB, SFTPC, and SFTPD, as well as NAPSA, a gene product that catalyzes the processing of the pro-form of surfactant proteins into mature proteins, were significantly downregulated in COVID-19 patient AT2 cells, while changes in other AT2-cell markers were minimal and insignificant (FIG. 20A and FIG. 20B). Pathway analysis revealed a significant enrichment for type-I and type-II IFN signaling, inflammatory programs, and cell death pathways in COVID-19 AT2 cells. Then, transcripts between AT2s from SARS-CoV-2 infected ex vivo cultures and COVID-19 patient lungs were directly compared. This revealed a striking similarity in upregulated transcripts. These include upregulation of chemokines and cytokines, including IFN ligands and their targets, indicating that AT2s derived from alveolospheres respond similarly to AT2s from human lungs after SARSCoV-2 infection.

### Example 10: AT2s Respond to Exogenous IFNs and Recapitulate Features Associated with SARSCoV-2 Infection

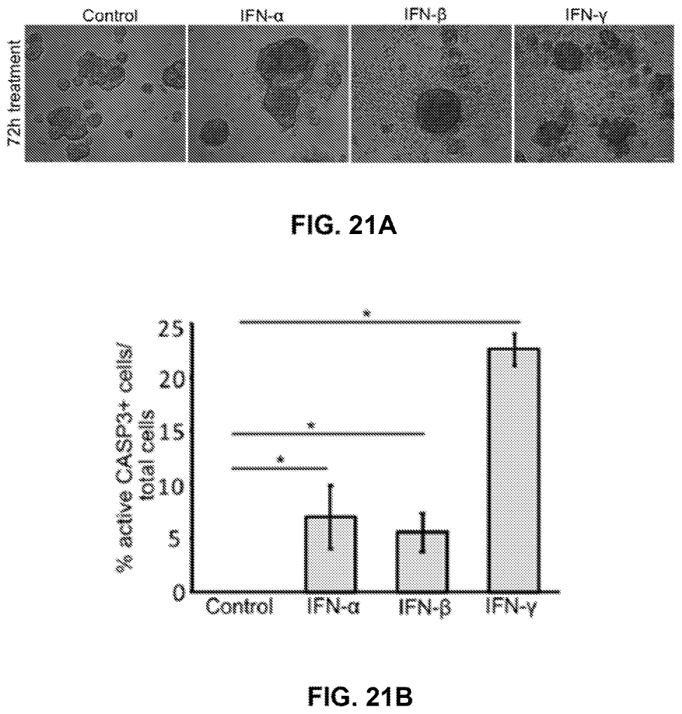

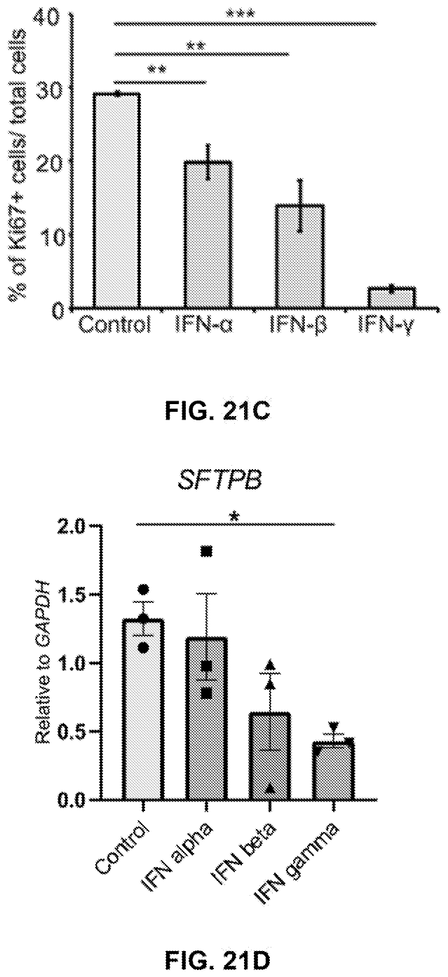

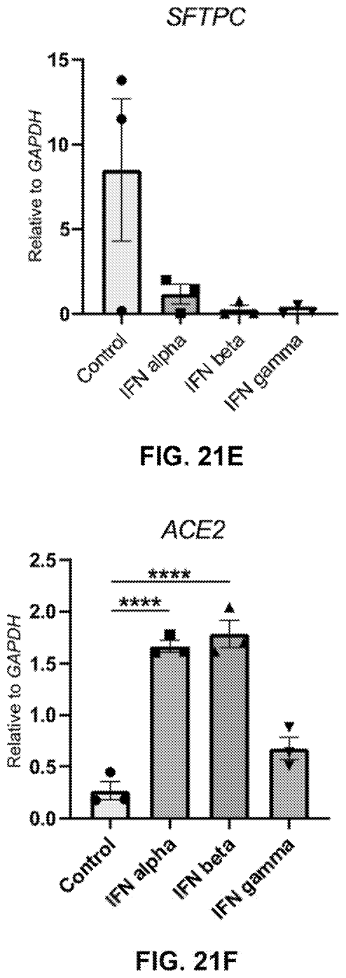

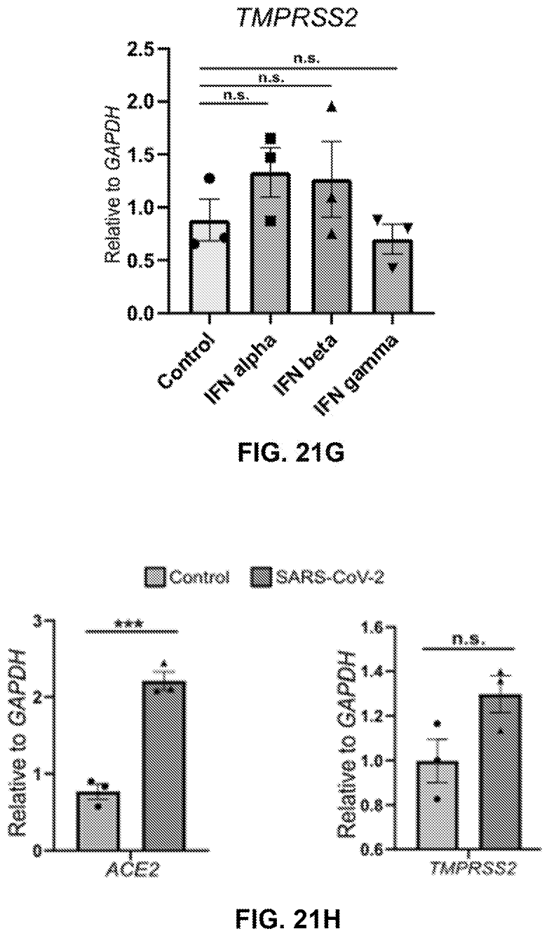

The transcriptome analysis revealed a striking similarity in interferon signatures in AT2s from alveolospheres and human lungs after SARS-CoV-2 infection. Previous studies have shown that IFNs induce cellular changes in a context dependent manner. For example, IFNa and IFNb provide protective effects in response to influenza virus infection in the lungs, whereas IFNg induces apoptosis in intestinal cells in response to chronic inflammation (Koerner et al., 2007, J. Virol. 81, 2025-2030; Takashima et al, 2019, Sci. Immunol. 4(42)). To test the direct effects of IFNs on AT2s, alveolospheres were treated with purified recombinant IFNa, IFNb, and IFNg in SFFF media and cultured them for 72 h. First, detached cells were observed in all treatments, with a maximal ˜3-fold increased effect in IFNg treated alveolospheres (FIG. 21A). Immunostaining for active caspase 3 revealed a significant induction of cell death in response to all IFN treatments, with a maximal effect with IFNg (FIG. 21B). In contrast, a significant reduction in cell proliferation in IFNb and IFNg treatments as revealed by immunostaining for Ki67, a marker for cell proliferation, was observed (FIG. 21C). Significantly, immunostaining revealed a reduction of SFTPB expression in alveolospheres treated with all IFNs compared to controls. A similar trend was observed for SFTPC and SFTPB transcripts as assessed by qRT-PCR. (FIG. 21D and FIG. 21E). These data are in accord with transcriptome data from AT2 alveolospheres after SARS-CoV-2 infection. Of note, treatment with IFNa, IFNb, and IFNg significantly enhanced the levels of ACE2, but not TMPRSS2 transcripts, which is in line with previous studies in other cell types (Hou et al., 2020; Ziegler et al., 2020) (FIG. 21F and FIG. 21G). A similar trend was observed in SARS-CoV-2 infected cells, suggesting a positive loop that involves IFNs and ACE2 which subsequently amplifies SARS-CoV-2 infection (FIG. 21H).

### Example 11: Pre-Treatment with IFNs Reduces SARS-CoV-2 Replication in Alveolospheres

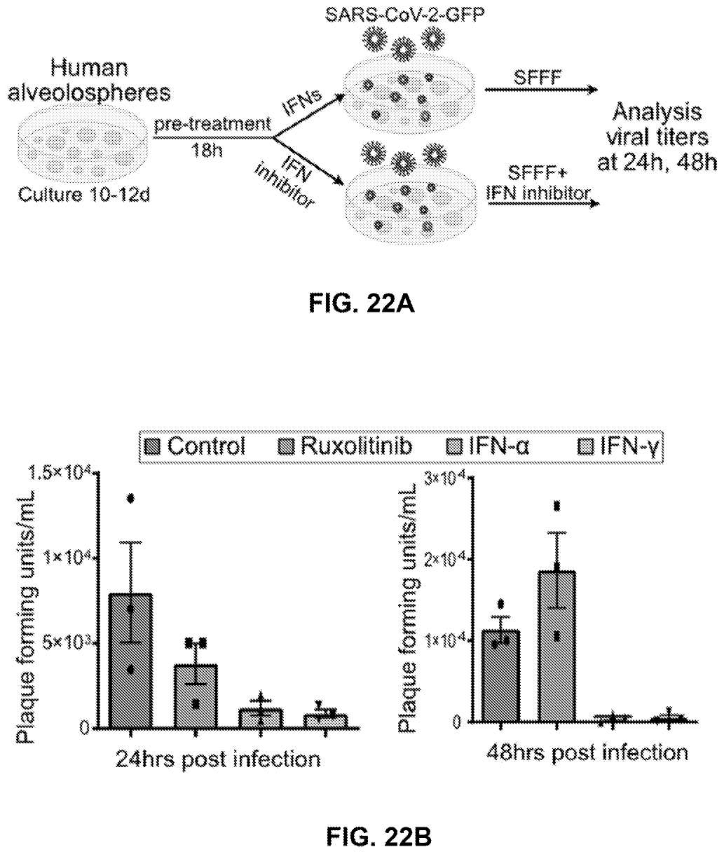

Recent studies suggested that pre-treatment with IFNs reduced SARS-CoV-2 replication in Calu-3 and Vero-2 cells. The effect of pre-treatment of alveolospheres with IFNs before viral infection was tested, since the above data from IFN treatments alone led to an increase in AT2 cell death. Therefore, alveolospheres were pretreated with a lower dose of IFNα and IFNγ (10 ng) for 18 h prior to viral infection (FIG. 22A). Subsequent plaque forming assays at 24 h and 48 h post infection revealed that pretreatment with IFNs significantly reduced the viral titers in alveolospheres (FIG. 22B). In addition, the effect of IFN signaling inhibition on viral replication was also tested. For this, alveolospheres were pretreated with Ruxolitinib, an inhibitor of IFN signaling, for 18 h and continued treatment following viral infection (FIG. 22A). Plaque forming assays revealed an increase in the viral replication (FIG. 22B). Taken together, these data suggest that pretreatment with IFNs gives a prophylactic effect whereas IFNs inhibition promotes viral replication.

Discussion

Using alveolosphere cultures, it was demonstrated that AT2s express SARS-CoV-2 receptor, ACE2, and are sensitive to virus infection. Transcriptome profiling further revealed the emergence of an “inflammatory state” in which AT2s activated the expression of numerous IFNs, cytokines, chemokines, and cell death related genes at later times post infection. These data are consistent with earlier studies showing delayed host innate immune responses after SARS-CoV (2003) infection, until later times (Menachery et al. 2014, mBio, 5(3): e01174-14), but also underscores the need for kinetic analyses of host responses at different times after infection. Both transcriptome and immunohistochemical analysis revealed a downregulation of surfactant proteins in SARS-CoV-2 infected alveolospheres. The finding that the Type-II IFN pathway is activated in AT2 cells ex vivo is surprising as typically it is the Type-I and Type-III pathways that are activated in cells by viral infection (Barrat et al., 2019, Nat. Immunol. 20, 1574-1583; Bartee et al., 2008, Curr. Opin. Microbiol. 11, 378-383). Significantly, these unexpected findings from alveolosphere-derived AT2s mirror responses in AT2 cells from COVID-19 patient lungs, further supporting the relevance of alveolosphere-derived AT2 for SARS-CoV-2 studies.

This study further provided evidence that pre-treatment with IFNs shows prophylactic effectiveness in alveolospheres.

There are several reasons why AT2 cells grown in organoid cultures are preferred over the currently used cell lines such as Calu-3, A549, Vero, and H1299. For example, A549 cells derived from a human lung adenocarcinoma have been widely used as surrogates for alveolar epithelial cells in viral infection studies. However, A549 cell line lacks the cardinal features of lung epithelial cells, including the ability to form epithelial tight junctions; they also harbor numerous genetic alterations (Osada et al., 2014, Genes Genomes 21, 673-683). More importantly, A549 cells do not express the SARS-CoV-2 receptor, ACE2, and viral infection studies rely on ectopic expression of this receptor. Accordingly, transformed cell lines do not faithfully recapitulate the native lung epithelial cells (Mason and Williams, 1980, Biochim. Biophys. Acta 617:36-50). In contrast, alveolar stem cell (AT2s) based alveolospheres are highly polarized epithelial structures that retain molecular, morphological features and maintain the ability to differentiate into AT1 cells under suitable conditions.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise.

The present disclosure shall control in the event there are any disparities between any definitions and/or description found in the cited references.

1. A type 2 alveolar epithelial cell culture medium comprising serum-free medium and an extracellular matrix component, wherein the culture medium is chemically defined and stroma free.

2. The medium of claim 1, wherein the serum-free medium and the extracellular matrix component are mixed at a ratio of about 1:1.

3. The medium of claim 3, wherein the extracellular matrix component is Matrigel™, Collagen Type I, Cultrex reduced growth factor basement membrane, Type R, or human type laminin.

4. The medium of claim 1, wherein the serum free medium comprises at least one growth nutrient selected from the group consisting of SB431542, CHIR 99021, BIRB796, Heparin, human EGF, FGF10, Y27632, Insulin-Transferrin-Selenium, Glutamax, B27, N2, HEPES, N-acetylcysteine, antibiotic-antimycotic in Advanced DMEM/F12, and combinations thereof.

5. The medium of claim 4 in which the serum free medium comprises SB431542, CHIR 99021, BIRB796, Heparin, human EGF, FGF10, Y27632, Insulin-Transferrin-Selenium, Glutamax, B27, N2, HEPES, N-acetylcysteine, and anti-anti in Advanced DMEM/F12.

6. A type 2 alveolar epithelial cell culture medium comprising a 1:1 mixture of a serum-free medium and a Matrigel, the serum-free media comprising 10 μM SB431542, 3 μM CHIR 9902, 1 μM BIRB796, 5 μg/ml Heparin, 50 ng/ml human EGF, 10 ng/ml mouse FGF10, 10 nM Y27632, Insulin-Transferrin-Selenium, 1% Glutamax, 2% B27, 1% N2, 15 mM HEPES, 1.25 mM N-acetylcysteine, and 1% anti-anti in Advanced DMEM/F12, and wherein the medium is stroma free.

7. The medium of claim 3, wherein the Matrigel is BD Biosciences #354230.

8. The medium of claim 1, wherein the medium is a type 2 alveolar epithelial cell culture expansion medium.

9. The expansion medium of claim 8, wherein the medium further comprises a cytokine selected from the group consisting of IL-1β, TNFα, and combinations thereof.

10-11. (canceled)

12. The expansion medium of claim 8, wherein the IL-1β or TNFα is at a concentration of about 10 ng/ml.

13. (canceled)

14. The medium of claim 1, wherein the medium is a maintenance medium, the maintenance medium comprising the expansion medium of any of claims 1-13, wherein the maintenance medium further comprises a bone morphogenetic protein (BMP) inhibitor.

15. The maintenance medium of claim 14, wherein the BMP inhibitor is selected from the group consisting of Noggin, DMH-1, chordin, gremlin, crossveinless, LDN193189, USAG-1 and follistatin, and combinations thereof.

16. (canceled)

17. The maintenance medium as in claim 15, wherein the Noggin is at a concentration of about 10 ng/ml, or DMH-1 is at concentration of about 1 μM.

18. (canceled)

19. The medium of claim 1, wherein the medium is a differential medium comprising the differentiation medium comprising at least one of the following growth medium components selected from the group consisting of ITS, Glutamax, Heparin, EFG, FGF10, and anti-anti in Advanced DMEM/F12 and/or combinations thereof.

20. The differentiation medium of claim 19, wherein the medium further comprises serum.

21. (canceled)

22. The differentiation medium of claim 19, wherein the medium comprises ITS, Glutamax, Heparin, EFG, FGF10, Fetal Bovine Serum, and 1% anti-anti in Advanced DMEM/F12.

23. The differentiation medium of claim 22, wherein the medium comprises ITS, Glutamax, about 5 μg/ml Heparin, about 5 ng/ml human EFG, about 1 ng/ml mouse FGF10, about 10% Fetal Bovine Serum, and about 1% anti-anti in Advanced DMEM/F12.

24. The differentiation medium of claim 19, wherein the differentiation medium does not contain inhibitors of TGFβ and p38 kinase.

25. The differentiation medium of claim 19, wherein the medium comprises IL-6.

26. The differentiation medium of claim 25, wherein the medium comprises 10 ng/mL to 50 ng/mL of IL-6.

27. The differentiation medium of claim 19, wherein the medium is a serum-free medium.

28. A chemically defined and stroma-free organoid culture system for the culturing, expansion, maintenance and/or differentiation of alveolar epithelial cells, the system comprising isolated alveolar epithelial cells cultured in a medium of claim 1.

29. (canceled)

30. A method of expanding, maintaining, and/or differentiating type 2 alveolar epithelial cell in ex vivo organoid cultures, the method comprising obtaining type 2 alveolar epithelial cells and culturing the cells in a medium of claim 1.

31-36. (canceled)

37. A method for identifying an agent capable of treating or preventing pathogen infections in an organoid culture, the method comprising
i) culturing the cells in the expansion medium of claim 1;
ii) inoculating the cells with a pathogen in an amount effective to infect the cells;
iii) contacting the cells with an agent; and
iv) determining whether the agent causes a reduction in the amount of the pathogen in the cells relative to a cell that has not been treated with the agent.

38. The method of claim 37, wherein step iii is optionally performed before step ii.

39. The method of claim 36, wherein the pathogen is a bacterium, virus, or fungus.

40. The method of claim 39, wherein the virus is 229E, NL63, OC43, HKU1, MERS-CoV, SARS-CoV, or SARS-CoV-2, an influenza-A virus, an influenza-B virus, or an enterovirus.

41-47. (canceled)

48. A kit comprising a chemically defined and stroma-free organoid culture system for the culturing, expansion, maintenance and/or differentiation of alveolar epithelial cells, the kit a medium of claim 1, and instructions for use.

49-50. (canceled)


SEQUENCE LISTING (50 sequences)

SEQ ID NO: 1 - DNA, 23 bp, Artificial Sequence
  Synthetic oligonucleotide
ggatgctagatatagtagagtgg

SEQ ID NO: 2 - DNA, 23 bp, Artificial Sequence
  Synthetic oligonucleotide
atcagagatcggaagaagaaaaa

SEQ ID NO: 3 - DNA, 21 bp, Artificial Sequence
  Synthetic oligonucleotide
ttgctaatatcgatggaggca

SEQ ID NO: 4 - DNA, 20 bp, Artificial Sequence
  Synthetic oligonucleotide
ccgaggagaaagggaagacc

SEQ ID NO: 5 - DNA, 20 bp, Artificial Sequence
  Synthetic oligonucleotide
tcaccctggcaagaatcgac

SEQ ID NO: 6 - DNA, 20 bp, Artificial Sequence
  Synthetic oligonucleotide
ccatgattcccaagggtgcg

SEQ ID NO: 7 - DNA, 19 bp, Artificial Sequence
  Synthetic oligonucleotide
cagccattctcctgtcggc

SEQ ID NO: 8 - DNA, 20 bp, Artificial Sequence
  Synthetic oligonucleotide
tccagagagcatccccagtc

SEQ ID NO: 9 - DNA, 18 bp, Artificial Sequence
  Synthetic oligonucleotide
ggcttccactgaccctgc

SEQ ID NO: 10 - DNA, 20 bp, Artificial Sequence
  Synthetic oligonucleotide
agatgtagcggacgagagga

SEQ ID NO: 11 - DNA, 20 bp, Artificial Sequence
  Synthetic oligonucleotide
gctgctcgtacaccttggag

SEQ ID NO: 12 - DNA, 23 bp, Artificial Sequence
  Synthetic oligonucleotide
aagatgaccactttggaaatgtg

SEQ ID NO: 13 - DNA, 20 bp, Artificial Sequence
  Synthetic oligonucleotide
gatggccccaatcacaggaa

SEQ ID NO: 14 - DNA, 20 bp, Artificial Sequence
  Synthetic oligonucleotide
ggcccggtccttttctttac

SEQ ID NO: 15 - DNA, 22 bp, Artificial Sequence
  Synthetic oligonucleotide
actcctcctctgggaatctgaa

SEQ ID NO: 16 - DNA, 19 bp, Artificial Sequence
  Synthetic oligonucleotide
acgccgcattgaccatcta

SEQ ID NO: 17 - DNA, 20 bp, Artificial Sequence
  Synthetic oligonucleotide
tggccttcaggtaatgcaga

SEQ ID NO: 18 - DNA, 19 bp, Artificial Sequence
  Synthetic oligonucleotide
ggtgactttggtgctaggc

SEQ ID NO: 19 - DNA, 18 bp, Artificial Sequence
  Synthetic oligonucleotide
agtgactcttccaaggcg

SEQ ID NO: 20 - DNA, 24 bp, Artificial Sequence
  Synthetic oligonucleotide
atttacagcaaccatgagtacaaa

SEQ ID NO: 21 - DNA, 21 bp, Artificial Sequence
  Synthetic oligonucleotide
tcccacactgtatttggtgtc

SEQ ID NO: 22 - DNA, 20 bp, Artificial Sequence
  Synthetic oligonucleotide
tgcaaccatgagtgagaaca

SEQ ID NO: 23 - DNA, 21 bp, Artificial Sequence
  Synthetic oligonucleotide
gataggccagtaggttgcaca

SEQ ID NO: 24 - DNA, 20 bp, Artificial Sequence
  Synthetic oligonucleotide
cagaactgcagggaaacagc

SEQ ID NO: 25 - DNA, 19 bp, Artificial Sequence
  Synthetic oligonucleotide
ggaaggattttctccaggg

SEQ ID NO: 26 - DNA, 21 bp, Artificial Sequence
  Synthetic oligonucleotide
aagtggcattcaaggagtacc

SEQ ID NO: 27 - DNA, 21 bp, Artificial Sequence
  Synthetic oligonucleotide
acgtggacaaaattggcttgc

SEQ ID NO: 28 - DNA, 19 bp, Artificial Sequence
  Synthetic oligonucleotide
ctccttctccacaagcgcc

SEQ ID NO: 29 - DNA, 18 bp, Artificial Sequence
  Synthetic oligonucleotide
gaaggcagcaggcaacac

SEQ ID NO: 30 - DNA, 21 bp, Artificial Sequence
  Synthetic oligonucleotide
tgagtcagcaaagaagtcaag

SEQ ID NO: 31 - DNA, 20 bp, Artificial Sequence
  Synthetic oligonucleotide
ggagtgggccatagcttaca

SEQ ID NO: 32 - DNA, 19 bp, Artificial Sequence
  Synthetic oligonucleotide
ttcgaggcacaaggcacaa

SEQ ID NO: 33 - DNA, 21 bp, Artificial Sequence
  Synthetic oligonucleotide
tggctgcttcagacacttgag

SEQ ID NO: 34 - DNA, 19 bp, Artificial Sequence
  Synthetic oligonucleotide
tcggagtcaacggatttgg

SEQ ID NO: 35 - DNA, 20 bp, Artificial Sequence
  Synthetic oligonucleotide
ttcccgttctcagccttgac

SEQ ID NO: 36 - DNA, 20 bp, Artificial Sequence
  Synthetic oligonucleotide
acaatcaccaccacaacgag

SEQ ID NO: 37 - DNA, 20 bp, Artificial Sequence
  Synthetic oligonucleotide
agcaaagaggtcctgatgga

SEQ ID NO: 38 - DNA, 20 bp, Artificial Sequence
  Synthetic oligonucleotide
ccgcctcagttgtcagcttc

SEQ ID NO: 39 - DNA, 22 bp, Artificial Sequence
  Synthetic oligonucleotide
acatcacagtggagggatagtg

SEQ ID NO: 40 - DNA, 21 bp, Artificial Sequence
  Synthetic oligonucleotide
gcttggtgttccttggtgttc

SEQ ID NO: 41 - DNA, 21 bp, Artificial Sequence
  Synthetic oligonucleotide
ccactgttgtgtgcttgagtc

SEQ ID NO: 42 - DNA, 20 bp, Artificial Sequence
  Synthetic oligonucleotide
ttgaggtcaatgaaggggtc

SEQ ID NO: 43 - DNA, 20 bp, Artificial Sequence
  Synthetic oligonucleotide
tcgtcccgtagacaaaatgg

SEQ ID NO: 44 - DNA, 22 bp, Artificial Sequence
  Synthetic oligonucleotide
gggagccttgaatacaccaaaa

SEQ ID NO: 45 - DNA, 21 bp, Artificial Sequence
  Synthetic oligonucleotide
tgtagcacgattgcagcattg

SEQ ID NO: 46 - DNA, 30 bp, Artificial Sequence
  Synthetic oligonucleotide
actggaacactaaacatagcagtggtgtta

SEQ ID NO: 47 - DNA, 11 bp, Artificial Sequence
  Synthetic oligonucleotide
aaccaaatgtg

SEQ ID NO: 48 - DNA, 22 bp, Artificial Sequence
  Synthetic oligonucleotide
aacaacagcattttggggtaag

SEQ ID NO: 49 - DNA, 22 bp, Artificial Sequence
  Synthetic oligonucleotide
ggctaccctcttgagtgcatta

SEQ ID NO: 50 - DNA, 22 bp, Artificial Sequence
  Synthetic oligonucleotide
gcaatttcatgctcatgttcac